SEQUENCE LISTING (50 sequences)

SEQ ID NO: 1 - protein, 303 aa, Arabidopsis thaliana
MKLTYQFFIFWFFLPFFAISGDDDYKNLIFKGCANQKSPDPTGVFSQNLKNLFTSLVSQSSQSSFASVTSGTDNTTAVIGVFQCRGDLQNAQCYDCVSKIPKLVSKLCGGGRDDGNVVAARVHLAGCYIRYESSGFRQTSGTEMLFRVCGKKDSNDPGFVGKRETAFGMAENGVKTGSSGGGGGGGGFYAGQYESVYVLGQCEGSLGNSDCGECVKDGFEKAKSECGESNSGQVYLQKCFVSYSYYSHGVPNIEPLSGGEKRQHTERTIALAVGGVFVLGFVIVCLLVLRSAMKKKSNKYDAY

SEQ ID NO: 2 - protein, 304 aa, Arabidopsis thaliana
MGFYSLKQLLLLYIIIMALFSDLKLAKSSSPEYTNLIYKGCARQRLSDPSGLYSQALSAMYGLLVTQSTKTRFYKTTTGTTSQTSVTGLFQCRGDLSNNDCYNCVSRLPVLSGKLCGKTIAARVQLSGCYLLYEISGFAQISGMELLFKTCGKNNVAGTGFEQRRDTAFGVMQNGVVQGHGFYATTYESVYVLGQCEGDIGDSDCSGCIKNALQRAQVECGSSISGQIYLHKCFVGYSFYPNGVPKRSSPYPSSGSSGSSSSSSSSGTTGKTVAIIVGGTAGVGFLVICLLFVKNLMKKKYDDY

SEQ ID NO: 3 - DNA, 900 bp, Arabidopsis thaliana
atgatcaagacaaagacgacgtcccttctctgtttccttctcacagccgttatactaatgaacccatcttcctcctcccccactgacaattacatatacgccgtttgttctccggctaaattctcccctagctccggctacgagacaaatcttaactccctcctctcttccttcgtcacctcaaccgcccaaacccgctacgccaacttcacagttcccaccgggaaaccggaaccaaccgttaccgtatacggtatttaccagtgccgcggcgacctagacccaaccgcctgctcgacgtgcgtatcaagcgccgtcgcacaggtcggagccttatgttcaaactcgtactccgggttcttgcagatggaaaattgtctgatccgttacgacaacaaatcgttccttggggttcaagacaaaacgttgatcctcaacaaatgtggacagccgatggaattcaacgaccaggatgcgttgaccaaagctagtgacgtcatcggttctttaggtaccggagatggatcttacaggacaggtggcaacgggaatgttcagggtgtggctcagtgctccggtgatctaagtacatcacagtgccaagattgcttgtctgatgcaatcggacggctcaaatctgattgtggaatggctcagggaggatatgtttacttgtcgaaatgttacgcgcgtttcagtgttggtggtagtcacgcgcgtcagaccccaggcccaaactttggtcatgaaggagagaagggcaataaggatgataatggagtgggaaaaacattggcgataataataggaatcgtcaccttaatcatcttgctcgttgtgtttctcgcttttgttggaaagtgttgtagaaaattacaagatgagaaatggtgtaaatga

SEQ ID NO: 4 - protein, 299 aa, Arabidopsis thaliana
MIKTKTTSLLCFLLTAVILMNPSSSSPTDNYIYAVCSPAKFSPSSGYETNLNSLLSSFVTSTAQTRYANFTVPTGKPEPTVTVYGIYQCRGDLDPTACSTCVSSAVAQVGALCSNSYSGFLQMENCLIRYDNKSFLGVQDKTLILNKCGQPMEFNDQDALTKASDVIGSLGTGDGSYRTGGNGNVQGVAQCSGDLSTSQCQDCLSDAIGRLKSDCGMAQGGYVYLSKCYARFSVGGSHARQTPGPNFGHEGEKGNKDDNGVGKTLAIIIGIVTLIILLVVFLAFVGKCCRKLQDEKWCK

SEQ ID NO: 5 - DNA, 900 bp, artificial
  polynucleotide encoding a modified PDLP5 protein
atgatcaagacaaagacgacgtcccttctctgtttccttctcacagccgttatactaatgaacccatcttcctcctcccccactgacaattacatatacgccgtttgttctccggctaaattctcccctagctccggctacgagacaaatcttaactccctcctctcttccttcgtcacctcaaccgcccaaacccgctacgccaacttcacagttcccaccgggaaaccggaaccaaccgttaccgtatacggtatttaccagtgccgcggcgacctagacccaaccgcctgctcgacgtgcgtatcaagcgccgtcgcacaggtcggagccttatgttcaaactcgtactccgggttcttgcagatggaaaattgtctgatccgttacgacaacaaatcgttccttggggttcaagacaaaacgttgatcctcaacaaatgtggacagccgatggaattcaacgaccaggatgcgttgaccaaagctagtgacgtcatcggttctttaggtaccggagatggatcttacaggacaggtggcaacgggaatgttcagggtgtggctcagtgctccggtgatctaagtacatcacagtgccaagattgcttgtctgatgcaatcggacggctcaaatctgattgtggaatggctcagggaggatatgtttacttgtcgaaatgttacgcgcgtttcagtgttggtggtagtcacgcgcgtcagaccccaggcccaaactttggtcatgaaggagagaagggcaataaggatgataatggagtgggaaaaacattggcgataataataggaatcgtcaccttaatcatcttgctcgttgtgtttctcgcttttgttggaaaggctgctagaaaattacaagatgagaaatgggctaaatga

SEQ ID NO: 6 - protein, 299 aa, artificial
  modified PDLP5 protein
MIKTKTTSLLCFLLTAVILMNPSSSSPTDNYIYAVCSPAKFSPSSGYETNLNSLLSSFVTSTAQTRYANFTVPTGKPEPTVTVYGIYQCRGDLDPTACSTCVSSAVAQVGALCSNSYSGFLQMENCLIRYDNKSFLGVQDKTLILNKCGQPMEFNDQDALTKASDVIGSLGTGDGSYRTGGNGNVQGVAQCSGDLSTSQCQDCLSDAIGRLKSDCGMAQGGYVYLSKCYARFSVGGSHARQTPGPNFGHEGEKGNKDDNGVGKTLAIIIGIVTLIILLVVFLAFVGKAARKLQDEKWAK

SEQ ID NO: 7 - DNA, 33 bp, artificial
  synthetic oligonucleotide primer
agtctcgagatgatcaagacaaagacgacgtcc

SEQ ID NO: 8 - DNA, 47 bp, artificial
  synthetic oligonucleotide primer
actgtcgactcatttacaccatttctcatcttgtaattttctacaac

SEQ ID NO: 9 - DNA, 32 bp, artificial
  synthetic oligonucleotide primer
caccgaatcatgatcaagacaaagacgacgtc

SEQ ID NO: 10 - DNA, 41 bp, artificial
  synthetic oligonucleotide primer
tcatcttgtaattttctagcagcctttccaacaaaagcgag

SEQ ID NO: 11 - DNA, 32 bp, artificial
  synthetic oligonucleotide primer
tcatttagcccatttctcatcttgtaattttc

SEQ ID NO: 12 - DNA, 6 bp, Arabidopsis thaliana
tgtctc

SEQ ID NO: 13 - DNA, 4 bp, Arabidopsis thaliana
tgtc

SEQ ID NO: 14 - protein, 11 aa, Arabidopsis thaliana
KNLMRKKHDDY

SEQ ID NO: 15 - protein, 11 aa, Arabidopsis thaliana
KNLMKKKYDDY

SEQ ID NO: 16 - protein, 14 aa, Arabidopsis thaliana
RSAMKKKSNKYDAY

SEQ ID NO: 17 - protein, 10 aa, Arabidopsis thaliana
KSLRKKGDDC

SEQ ID NO: 18 - protein, 5 aa, Arabidopsis thaliana
RNSMH

SEQ ID NO: 19 - protein, 14 aa, Arabidopsis thaliana
GKCCRKLQDEKWCK

SEQ ID NO: 20 - protein, 9 aa, Arabidopsis thaliana
RGVCSRGGK

SEQ ID NO: 21 - protein, 11 aa, Arabidopsis thaliana
AKSCERGKGGK

SEQ ID NO: 22 - protein, 302 aa, Populus trichocarpa
MSRTITMAPLISLLTIALLTTPAISSVDTFVYGGCSQVKYTPGSPYESNVNSLLTSLVNSATFTIYNNFTIKSPTSQDTLYGLFQCRGDLSNGDCASCVARAVSQLGTLCLDSTGGALQLDGCFVKYDNTTFLGVEDKTEVLKKCGPLIAYDSDELNRRDAVMDYLGTSDGSSKPFRIGGSGDISAVAQCVQDLSASECQDCLSEVVGRLKTYCGAAASGDMYLAKCYVRFSKAGAHSHGGNVDHDENDEVEKTLAILVGLIAGVALLIVFLAFLRKACGKGKCKLLVYLSLHLYSSLYCVP

SEQ ID NO: 23 - protein, 297 aa, Prunus persica
MSLTSHQTLVFLWVFLLATVSFLATPSASAINSFVFGGCSQQKYLPGSPYESKVNSLLTSLVNSAMFTTYNNFTIPGSSSQDTVYGLFQCRGDLSNNDCAQCVARSVSQLGNLCLNSCGGALQLEGCFIKYDNSTFLGVEDKTVVIKKCGQSIGFDSDVLTRRDAVLGYLGTGDGTYRPYRVSGSGNVQGVAQCVGDLSPSECQDCLSEAIAQLKSGCGPSAWGDMFLAKCYARYSQGGYHTNGGHDYHNDDDDDDDDDELEKTLAILIGVIAGVALLVVFLSYFRKYLCEEEKCGK

SEQ ID NO: 24 - protein, 310 aa, Brassica rapa
MFKTTTTLLCFFLTSVILMVPTSSAATDNPTYSASTDTFIYANCSPAKFSPGSAYETNLKSLLSSLVTSTVLNRYNNLTVPFGSGVKPGPDVTVYGLFQCSVDLDPTSCSSCVSRAIALVGNTCPNSYSVFLQMQNCLVRYDKSSFFGVQDKTVMLKKCGQPMGFYDQDALTRVSDVIGSLGSGSEPDRTRMNGDVLGMAQCTEDLSPAQCQDCLTDAIGQLRSDCLMAQGGYVYLSKCYARFSFGGSHARQTPNSNFGGEKYDKDDDDNNIGKTLVIIIGIITLVILLVLLLAFLGKKLRKLQDDKCCR

SEQ ID NO: 25 - protein, 316 aa, Brassica rapa
MASLRNTLSLVFCLLAATGPWLCSATSATSATDTFVYGGCSQQKFSPASPYESNLNSLLTSLVNSATYSSYNNFTIMGSSSSDTARGLFQCRGDLSMPDCATCVARAVSQVGPLCPYTCGGALQLAGCYIKYDNVSFLGQEDKTVVLKKCGPSEGYNTEGISRRDAVLTELLGGGGYFRAGGSSDVQGMGQCVGDLTVSECQDCLGTAIGRLKNDCGTAVFGDMFLTKCYARYSTDGGKYNAKSHNYKTNYGGERTFAIIIGLLAAVVLLIIFLLFLRGVCSRGGNQLFFLALSSILILFNFFHNIENTHKHCILG

SEQ ID NO: 26 - protein, 343 aa, Brassica rapa
MEPHIRAPFFTLILFKARASLLYTTSIFTKLSHFFLYLSKKKKVNMSKTKTTLLSFLVTVVILMNPSASNPTDGYIYAVCSPAKFSPGSGYEANLNSLLSSFVSSTVQIRYVNFTVPNKKPEPGVTVYGLYQCRGDLDPTSCSTCVSRANAQVGTLCSNSYSGFLQMDSCLVRYDNTSFFGVQDKTLRLNKCGQAMELNDQDALTRISDVIGSLGSGSDSYRTGGNGDVQGTAQCMGDLSTAQCQDCLSDAIGRLKSDCGMAQGGYVYLSKCYARFSVASSHARQTPNYNAGEKDDKDDDDKRVGKTLAIIIGMVTLIILLVVFLAFVGKQCGKYQEDNSCKY

SEQ ID NO: 27 - protein, 342 aa, Brassica rapa
MEPHIRAPFFTLILFKARASLLYTTSIFTKLSHFFLYLSKKKKVNMSKTKTTLLSFLVTVVILMNPSASNPTDGYIYAVCSPAKFSPGSGYEANLNSLLSSFVSSTVQIRYVNFTVPNKKPEPGVTVYGLYQCRGDLDPTSCSTCVSRANAQVGTLCSNSYSGFLQMDSCLVRYDNTSFFGVQDKTLRLNKCGQAMELNDQDALTRISDVIGSLGSGSDSYRTGGNGDVQGTAQCMGDLSTAQCQDCLSDAIGRLKSDCGMAQGGYVYLSKCYARFSVASSHARQTPNYNGEKDDKDDDDKRVGKTLAIIIGMVTLIILLVVFLAFVGKQCGKYQEDNSCKY

SEQ ID NO: 28 - protein, 287 aa, Populus trichocarpa
MSRILTITTLISLLTITVLTAPATSSTDSFVFGGCSQLKYTPGSPYESNVNLLLTSLVSSAAFTTYNNFTIKSPTPQDTLYGLFQCRGDLSNGDCASCVARAVSQLGTLCLDSSGGALQLEGCFVKYDNTTFLGVEDKTEVLHKCGPLIGYDSDELNRRDAVLGYLGTSDGSYRPFRVGGSGDVSSVAQCVQDLSASECQDCLSEAIGRLKTVCGPAVWGDLYLAKCFVRFSKAGASSNGGNGHDNSGNDEVEKTLAILIGLIAAVALLIVFLSFFRKVCERERGCK

SEQ ID NO: 29 - protein, 300 aa, Arabidopsis lyrata
MFKTKTTTSLLCFLLTAVILMNPSSSSPTDNYIYAVCSPAKFSPSSGYETNLNSLLSSFVSSTAQSRYANFTVPTGKPEPTVTVYGLYQCRGDLDPTACSTCVSSAVAQVGTLCSNSYSGFLQLENCLIRYDNKSFLGVQDKTLILNKCGQAMDFNDQDALTKVSDVIGSLGSGDGPYRNGGNGNVQGVAQCSGDLSTSQCQDCLSDAIGRLKSDCGMAQGGYVYLSKCYARFSVGGSHARQTPGPNFGHEGEKDNKDDNGVGKTLAIIIGIVTLIILLVVFLAFLGKQCRKLQDEKWCK

SEQ ID NO: 30 - protein, 14 aa, artificial
  consensus sequence of a cytosolic C-terminal sequence of a modified PDLP5 protein
XKXXXXXQXXXXXX

SEQ ID NO: 31 - DNA, 22 bp, artificial
  synthetic oligonucleotide primer
tttgcatagacgaaaaacatgg

SEQ ID NO: 32 - DNA, 21 bp, artificial
  synthetic oligonucleotide primer
tggatcttacaggacaggtgg

SEQ ID NO: 33 - DNA, 33 bp, artificial
  synthetic oligonucleotide primer
ccttttcagaaatggataaatagccttgcttcc

SEQ ID NO: 34 - DNA, 24 bp, artificial
  synthetic oligonucleotide primer
ggaagaccataacccttgaggttg

SEQ ID NO: 35 - DNA, 20 bp, artificial
  synthetic oligonucleotide primer
tcttagcaccaccacggaga

SEQ ID NO: 36 - DNA, 20 bp, artificial
  synthetic oligonucleotide primer
ccgctacgccaacttcacag

SEQ ID NO: 37 - DNA, 23 bp, artificial
  synthetic oligonucleotide primer
cttctctccttcatgaccaaagt

SEQ ID NO: 38 - DNA, 28 bp, artificial
  synthetic oligonucleotide primer
gttcactcaaatctataataggcatagg

SEQ ID NO: 39 - DNA, 23 bp, artificial
  synthetic oligonucleotide primer
cgacaaattgtggaactctttca

SEQ ID NO: 40 - DNA, 22 bp, artificial
  synthetic oligonucleotide primer
ggtagaggctaacgaattcaca

SEQ ID NO: 41 - DNA, 24 bp, artificial
  synthetic oligonucleotide primer
gtgcgtctatccattacaactttc

SEQ ID NO: 42 - DNA, 24 bp, artificial
  synthetic oligonucleotide primer
tgcattggtacacaggtgagggaa

SEQ ID NO: 43 - DNA, 24 bp, artificial
  synthetic oligonucleotide primer
agccgttgcatcttggtattgctg

SEQ ID NO: 44 - DNA, 24 bp, artificial
  synthetic oligonucleotide primer
cacaatgtttggcgggattggtga

SEQ ID NO: 45 - DNA, 24 bp, artificial
  synthetic oligonucleotide primer
tgtacttcctttccggtggagcaa

SEQ ID NO: 46 - DNA, 3089 bp, Arabidopsis thaliana
caaaaaccaattggaagatacatctaaatattaccttagatattacacattaaattttatcaaaaaaataaaatatatattacacattaaacatgaaataaataaaaaagaagaagactggatcagacattatgcttattgtggtgtctaatctaacatacacatgtgtcgtgttacattcttttagttcccttttcaaatagaaaaacttttgtttaaagggacagccaagtgggtgagtttaaaccttttattcctcaaaataattagatcctgacaaattatgtcttaatccacaactaatctactttcatatatggcattgatcaatatctttatacactttcaggatgtaatggtctctaactctctctatatataagatatgctcgaatatgtagcaacaattgtaaacgagtatcatataatcaataagactttaaaaaataatcagaatcagattcttactaaccaattactgatgcgtaagaagagaaaaggtgaaaagatcataattatatctgatcttgctatgaacatattcatgtgaaaatagacagactttgatacgaagaaaactaaaacagagggaaattgatcttgtcagtttgtactataacttttgaattcttgctagatattttactctagcctctaaatgaaaacgtaaattttgaaggtaaacataagacgatcctaattataatttgtaagctaagattctcatataaataatatcagattagttcactcaaatctataataggcatagggatattggatgtctccacagattttaatcttgaaagagttccacaatttgtcgaagaaacgttttccttttggaattgacgaatgagtcaacttaattaggttagtctaaatacaagaaaattatcgagtgcccattcgaatttttttattatttttcaggaaatgaccaaagacttattagaagtcaagcacgaaattactcaccaaaatcccttaatctctgcattagtttatttctccctaaatttatattttctccatctaaatgattacggatccacctgccaaggaaaaaaaaataactgtgttacgaaaaaattaaaaatcaatacttggttggaaaactaaaactaaaaccgaacaaaaatccaaatatgacattcctattctaattcaacaatagaattaattattagcgttggaacaatctatatatagatattaaagctttgtgtgtgtaagatagaagagttcttaaaagtctatttgtgtgggtaagccattgatcgtttagtgactaaatgatacaacttctaatcccctttatcaaactttatccagtctttgtttagctgtacttttcactgatatattttctcttcttatgttacgtgaaaataaaataatactattgaaaagtaatttgattgatcaattccaccatgcaattaattaccagaactttctgctttcaattatatcttcttaattaattggttattatttcaacttcataactgaatagaatcccttatcgacaaaatgattactcttgtattgttacattattgtcattgtttccttaataattatcaatgatctagtatcataaatgttaccttctatagttaatagttcatgtactaagtactaaaacatgtgtagtatgaaaaaatacaacatccatgttgtaattaatttatagatagattcaagtatacgtacaacaataaattcttttgcagccgtaacgatttttttattttttattttttattgaataccagtaaattctgggccgaagcccgtaatcccctcaggttcgaaaccacagcatgtaatattagtttcgtcccaagcgtcattcgaactggcgacctctaagattcgcctaaggtctttactaattgagctacaatggataataaaagaatctaaccttccaacaaaaagaaatagtattactaaagtcaataagaacaatctatagccaaagaaaaggtctatgggtgttgtggttataagaactaaacaaaagtcggtcaaataacattattgtcttctctattcgttcaccggtcccgtaactgaactttgacgttggcagcctagttacttgattgatttctcatcatactaataaaaataataaaatgttgatacaacccttacataaatgtatacaacatctcgaccagcacgccaatatccctttctatataattaaaattcatcttctaaaatcattttcaagtaccattgcctatccaaactctatacgctatactacatgtaatatattaatgaccataactcgttttcatgacgtatacgtcgtcattgctcggtattattcgtattgctttttttttctgatgatatagaaaaacaaaaaaaaagacctactattgtcaatttttatatacacacacataatgcgagaattgcttcgtcactcctatcatctgatataagaaaaataaactagttgaatgcaaattttcatatctttttgtgttgattatatatacataatacatgaataaagaagttggttttttattaatttcgtggtaagtccgccgatttggcctttctatacgcattcaaacttcacaatactataagaaattaactcctcagttttattgccttcttgataagtggcaactggcaagtaagggtaattagtagtaacggtagaggctaacgaattcacacgagtcactgtcaaaataaaaaattgaattcaaatgacatgagtaaggatagattaacaaaaagaaagttgtaatggatagacgcacattgccaaattccacattatagctttaaaaaaaaaagtctgaacgaagaaagagttgcaattatttagtaatttcaatgatatggaacttgcaaattgtggaaattcttaagaacgtccccaaccaaaaaccaaaaaaaaaaaaaccaaaagcatgcaacatggaaccacatataagagtacccttctttacgtcaattatctttaaagcaagtcttcgctcttcaatttatattttaccaaatcatcttctctctttctctatctctcaaaacaaaaagtaaccatg

SEQ ID NO: 47 - DNA, 4 bp, Arabidopsis thaliana
tata

SEQ ID NO: 48 - DNA, 4 bp, Arabidopsis thaliana
gaca

SEQ ID NO: 49 - DNA, 6 bp, Arabidopsis thaliana
tgtctc

SEQ ID NO: 50 - DNA, 6 bp, Arabidopsis thaliana
gagaca

(19) United States
(12) Patent Application Publication
(10) Pub. No.: US 2016/0272995 A1
(43) Pub. Date: Sep. 22, 2016

(54) MANIPULATION OF PLASMODESMAL CONNECTIVITY TO IMPROVE PLANT YIELD AND FITNESS

(71) Applicant: UNIVERSITY OF DELAWARE, Newark, DE (US)

(72) Inventor: Jung-Youn Lee, Newark, DE (US)

(73) Assignee: UNIVERSITY OF DELAWARE, Newark, DE (US)

(21) Appl. No.: 15/021,184

(22) PCT Filed: Sep. 12, 2014
(86) PCT No.: PCT/US14/55440

§ 371 Date: Mar. 10, 2016

Related U.S. Application Data
(60) Provisional application No. 61/876,806, filed on Sep. 12, 2013.
(60) Provisional application No. 61/884,277, filed on Sep. 30, 2013.
(60) Provisional application No. 61/893,376, filed on Oct. 21, 2013.

(51) Int. Cl. C12N 15/82; C07K 14/415

(52) U.S. Cl. CPC C12N 15/8273; C07K 14/415; C12N 15/8261; C12N 15/827; C12N 15/8241; C12N 15/8205; C12N 15/8207

(57) ABSTRACT

Plants are provided that express a modified plasmodesmata localized protein 5 (PDLP5). The modified PDLP5 protein may modify plasmodesmal connectivity. The plant may have at least one improved agronomic characteristic; for example, it may exhibit increase tolerance to stress, such as heat, cold or drought. The plant may exhibit at least one trait selected from the group consisting of: increased drought tolerance, increased yield, increased biomass, increased cold tolerance, early flowering and altered root architecture. Also included are related polypeptides, polynucleotides, recombinant DNA constructs, plant seeds and other plant parts that contain a modified PDLP5 protein, as well as methods of making and using the plants, seeds, polypeptides, polynucleotides, and recombinant DNA constructs.

### CONTINUING APPLICATION DATA

This application claims the benefit of U.S. Provisional Application Serial No. 61/876,806, filed Sep. 12, 2013, U.S. Provisional Application Serial No. 61/884,277, filed Sep. 30, 2013, and U.S. Provisional Application Serial No. 61/893,376, filed Oct. 21, 2013, each of which is incorporated by reference herein.

### GOVERNMENT FUNDING

This invention was made with government support under grant numbers RR015588, RR031160 and GM103519-03 awarded by the National Institutes of Health, and under grant number IOS-0954931, awarded by the National Science Foundation. The government has certain rights in the invention.

### BACKGROUND

Plasmodesmata (PD) facilitate cell-to-cell communication throughout plant tissues by serving as symplastic conduits through which small molecules such as ions, metabolites, and hormones can diffuse from one cell to another, thereby allowing the intercellular coordination of biochemical and physiological processes.

A family of PD-localized proteins (PDLP) has been identified as affecting PD permeability. PD-located protein 1 (PDLP1) was first identified by Thomas et al. (2008 PLoS Biol. 6:e7), and additional members of the PDLP family have been identified based on sequence homology to PDLP1. PDLPs range from 30 to 35 kD in predicted size and are composed of two conserved Cys-rich repeats containing DUF26 domains at the N terminus, followed by a transmembrane domain (TMD) and a very short cytoplasmic tail at the C-terminus. The DUF26 domain, a plant specific protein module, is characterized by conserved Cys residues and is found in a plant protein superfamily including Cys-rich receptor-like kinases (CRKs) and Cys-rich secretory proteins. The eight PDLP family members (PDLP1-8) contain DUF26 domains and a TMD, which anchors the proteins to the membrane, but lack the cytosolic kinase domain.

### SUMMARY OF THE INVENTION

Abiotic stress is the primary cause of crop loss worldwide, causing average yield losses of more than 50% for major crops. Among the various abiotic stresses, drought is the major factor that limits crop productivity worldwide. Biotic stress also impacts plant health and reduces the yield of the cultivated plants or affects the quality of the harvested products. The development of plants with increased tolerance to stress, both by conventional breeding methods and by genetic engineering, is an important strategy to meet crop production demands.

The present invention provides a modified PDLP5 protein that, when expressed in a plant, has a positive effect on one or more agronomic characteristics of the plant. The plant contains a recombinant DNA construct that contains a polynucleotide operably linked to at least one regulatory element, wherein the polynucleotide encodes the modified PDLP5 protein. The plant can be a transgenic plant. Also included in the invention are seeds, other plant parts, and plant progeny that include the recombinant DNA construct that contains polynucleotide encoding the modified PDLP5 protein. The proteins, polypeptides, polynucleotides and recombinant DNA constructs set for the herein are included in the invention, as are methods of making or using them. Methods of making or using a plant, seed, or other plant part that includes a recombinant DNA construct that encodes the modified PDLP5 protein are also encompassed by the invention. A plant that contains a recombinant DNA construct of the invention may be resistant to one or more abiotic or biotic stresses. An example of an agronomic characteristic that can be enhanced by expression of the modified PDLP5 protein in the plant is drought resistance. In some embodiments, the plant may exhibit better drought tolerance, better cold tolerance, faster vegetative growth, earlier flowering, and/or better yield, compared to a control plant that does not contain the recombinant DNA construct. In some embodiments, the plant may exhibit an alteration in root architecture compared to a control plant that does not contain the recombinant DNA construct. The alteration in root architecture can take the form of more extensive root architecture.

In one embodiment, the invention provides a plant comprising a recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, and wherein said plant exhibits at least one trait selected from the group consisting of: increased drought tolerance, increased yield, increased biomass, increased cold tolerance, early flowering and altered root architecture, when compared to a control plant not comprising said recombinant DNA construct. The plant can be selected from the group consisting of maize, soybean, sunflower, sorghum, canola, wheat, alfalfa, cotton, rice, barley, millet, sugar cane and switchgrass, for example. The invention also includes the seed of the plant comprising the recombinant DNA construct, wherein said seed comprises a recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, and wherein a plant produced from said seed exhibits at least one trait selected from the group consisting of: increased drought tolerance, increased yield, increased biomass, increased cold tolerance, early flowering and altered root architecture, when compared to a control plant not comprising said recombinant DNA construct.

In one embodiment, the invention provides a method of increasing drought tolerance in a plant, wherein the method comprises:

(a) introducing into a plant cell, for example a regenerable plant cell, a recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail;

(b) regenerating a transgenic plant from the regenerable plant cell of (a), wherein the transgenic plant comprises in its genome the recombinant DNA construct; and

(c) obtaining a progeny plant derived from the transgenic plant of (b), wherein said progeny plant comprises in its genome the recombinant DNA construct and exhibits increased drought tolerance when compared to a control plant not comprising the recombinant DNA construct.

In one embodiment, the invention provides a method of producing a plant that exhibits at least one trait selected from the group consisting of increased drought tolerance, increased yield, increased biomass, increased cold tolerance, early flowering and altered root architecture, wherein the method comprises growing a plant from a seed comprising a recombinant DNA construct, wherein the recombinant DNA construct comprises a polynucleotide operably linked to at least one regulatory element, wherein the polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, wherein the plant exhibits at least one trait selected from the group consisting of: increased drought tolerance, increased yield, increased biomass, increased cold tolerance, early flowering and altered root architecture, when compared to a control plant not comprising the recombinant DNA construct.

In one embodiment, the invention provides a method of producing a seed, the method comprising the following:

(a) crossing a first plant with a second plant, wherein at least one of the first plant and the second plant comprises a recombinant DNA construct, wherein the recombinant DNA construct comprises a polynucleotide operably linked to at least one regulatory element, wherein the polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail; and

(b) selecting a seed of the crossing of step (a), wherein the seed comprises the recombinant DNA construct.

The invention further provides a plant grown from the seed, wherein the plant exhibits at least one trait selected from the group consisting of: increased drought tolerance, increased yield, increased biomass, increased cold tolerance, early flowering and altered root architecture, when compared to a control plant not comprising the recombinant DNA construct.

In one embodiment, the invention provides a method of producing oil or a seed by-product, or both, from a seed, the method comprising extracting oil or a seed by-product, or both, from a seed that comprises a recombinant DNA construct, wherein the recombinant DNA construct comprises a polynucleotide operably linked to at least one regulatory element, wherein the polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail. The seed can be obtained from a plant that comprises the recombinant DNA construct and exhibits at least one trait selected from the group consisting of: increased drought tolerance, increased yield, increased biomass, increased cold tolerance, early flowering and altered root architecture, when compared to a control plant not comprising the recombinant DNA construct. The oil or the seed by-product, or both, may comprise the recombinant DNA construct.

In one embodiment, the invention provides a method of selecting for a plant that exhibits at least one trait selected from the group consisting of: increased drought tolerance, increased yield, increased biomass, increased cold tolerance, early flowering and altered root architecture, wherein the method comprises:

(a) obtaining a transgenic plant, wherein the transgenic plant comprises in its genome a recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail;

(b) growing the transgenic plant of part (a) under conditions wherein the polynucleotide is expressed; and

(c) selecting the transgenic plant of part (b) that exhibits at least one trait selected from the group consisting of: increased drought tolerance, increased yield, increased biomass, increased cold tolerance, early flowering and altered root architecture, when compared to a control plant not comprising the recombinant DNA construct.

The altered root architecture may be an increase in root mass. The increase in root mass may be at least 5%, when compared to a control plant not comprising the recombinant DNA construct.

In any of the methods provided herein, the plant may be selected from the group consisting of maize, soybean, sunflower, sorghum, canola, wheat, alfalfa, cotton, rice, barley, millet, sugar cane and switchgrass.

In one embodiment, the invention provides an isolated polynucleotide comprising:

(a) a nucleotide sequence encoding a modified PDLP5 protein with drought tolerance activity, wherein the modified PDLP5 protein has an amino acid sequence of at least 80% and less than 100% sequence identity when compared to SEQ ID NO:4, based on the Clustal V method of alignment with pairwise alignment default parameters of KTUPLE=1, GAP PENALTY=3, WINDOW=5 and DIAGONALS SAVED=5; or

(b) the full complement of the nucleotide sequence of (a).

The amino acid sequence of the modified PDLP5 protein may comprise SEQ ID NO:6. The nucleotide sequence may comprise SEQ ID NO:5.

Also provided by the invention is a plant or seed comprising a recombinant DNA construct, wherein the recombinant DNA construct comprises the polynucleotide operably linked to at least one regulatory element.

The plant or seed may be selected from the group consisting of maize, soybean, sunflower, sorghum, canola, wheat, alfalfa, cotton, rice, barley, millet, sugar cane and switchgrass.

The modified PDLP5 protein may comprise a modification at a cysteine residue in the C-terminal cytoplasmic tail, compared to a wild-type PDLP5 protein. The modification in the C-terminal cytoplasmic tail may comprise a modification of at least one cysteine residue selected from C288, C289, and C298 of A. thaliana PDLP5 (SEQ ID NO:4) or an analogous cytosolic cysteine residue in a homologous PDLP5 protein, at least two cysteine residues selected from C288, C289, and C298 of A. thaliana PDLP5 (SEQ ID NO:4) or analogous cytosolic cysteine residues in a homologous PDLP5 protein, or all three cysteine residues C288, C289, and C298 of A. thaliana PDLP5 (SEQ ID NO:4) or analogous cytosolic cysteine residues in a homologous PDLP5 protein. The modification may comprise an amino acid substitution or a deletion. The amino acid substitution may be a substitution with alanine.

In one embodiment, the invention provides a recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified

PDLP5 protein has 0, 1 or 2 cysteines in the cytosolic C-terminal tail. The polynucleotide may comprise an isolated polynucleotide comprising:

(a) a nucleotide sequence encoding a modified PDLP5 protein with drought tolerance activity, wherein the modified PDLP5 protein has an amino acid sequence of at least 80% and less than 100% sequence identity when compared to SEQ ID NO:4, based on the Clustal V method of alignment with pairwise alignment default parameters of KTUPLE=1, GAP PENALTY=3, WINDOW=5 and DIAGONALS SAVED=5; or

(b) the full complement of the nucleotide sequence of (a).

The amino acid sequence of the polypeptide may comprise SEQ ID NO:6. The nucleotide sequence may comprise a polynucleotide sequence encoding SEQ ID NO:6, such as SEQ ID NO:5.

The regulatory element included in the DNA construct may comprise a promoter. The promoter may be selected from the group consisting of a constitutive promoter, a tissue-specific promoter, and a physically inducible promoter that stimulates expression in response to exposure to plant stress.

The modified PDLP5 protein may exhibit semi-dominant negative gain-of-function activity when compared to a wild-type PDLP5 protein.

The modified PDLP5 protein may be PDLP5-m5 (SEQ ID NO:6).

In one embodiment, the plant may comprise an endogenous PDLP5 protein, and the modified PDLP5 protein may exhibit a semi-dominant negative gain-of-function activity.

In one embodiment, the invention provides a plant or plant seed comprising a recombinant DNA construct encoding PDLP5-m5 (SEQ ID NO:6).

In one embodiment, the invention provides a method of producing a plant that exhibits at least one trait selected from the group consisting of increased drought tolerance, increased yield, increased biomass, increased cold tolerance, early flowering and altered root architecture, wherein the method comprises:

(a) introducing into a plant cell a recombinant DNA construct comprising a polynucleotide operably linked to at least one regulatory element, wherein said polynucleotide encodes a modified PDLP5 protein comprising PDLP5-m5 (SEQ ID NO:6);

(b) growing a transgenic plant from the plant cell of (a), wherein the transgenic plant comprises in its genome the recombinant DNA construct; and

(c) obtaining a progeny plant derived from the transgenic plant of (b), wherein said progeny plant comprises in its genome the recombinant DNA construct and exhibits at least one trait selected from the group consisting of increased drought tolerance, increased yield, increased biomass, increased cold tolerance, early flowering and altered root architecture when compared to a control plant not comprising the recombinant DNA construct.

In one embodiment, the invention provides a method of producing a plant that exhibits at least one trait selected from the group consisting of increased drought tolerance, increased yield, increased biomass, increased cold tolerance, early flowering and altered root architecture, wherein the method comprises growing a plant from a seed comprising a recombinant DNA construct, wherein the recombinant DNA construct comprises a polynucleotide operably linked to at least one regulatory element, wherein the polynucleotide encodes a modified PDLP5 protein comprising PDLP5-m5 (SEQ ID NO:6); wherein the plant exhibits at least one trait selected from the group consisting of: increased drought tolerance, increased yield, increased biomass, increased cold tolerance, early flowering and altered root architecture, when compared to a control plant not comprising the recombinant DNA construct.

In one embodiment, the invention provides a method of making a plant wherein the endogenous PDLP5 has been modified, wherein the method comprises the steps of:

(a) introducing a mutation into the endogenous PDLP5 gene; and

(b) detecting the mutation.

Steps (a) and (b) may be done using a Targeting Induced Local Lesions IN Genomics (TILLING) method, and the mutation may be effective in modifying activity of the endogenous PDLP5 gene. Alternatively or additionally, step (a) may be performed using clustered regularly interspaced short palindromic repeats (CRISPR) technology. The mutation can be a site-specific mutation. The mutation can comprise a modification in a codon for a cysteine residue in the C-terminal cytoplasmic tail of the endogenous PDLP5. The mutation can result in a modified PDLP5 protein in which 1, 2, 3 or more cysteine residues in the cytosolic C-terminal tail have been changed or removed.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows a model illustrating role of plasmodesmata in the symplastic pathway in plants. (a) A schematic diagram of a plasmodesma illustrating the ultrastructure and cell-to-cell trafficking of diffusible signaling molecules. Spheres and short rods represent hypothetical proteinaceous and filamentous components observed within plasmodesma. (b) Plasmodesmal-mediated signaling among symplastically connected cells. Some signals move only into cells adjacent to the original cell that generated them for local communication, whereas systemic signals move farther to reach phloem for long-distance communication. (c) Environmental signals (e.g. day length or light intensity) or challenges (e.g. biotic stresses caused by microbial pathogen infection) perceived in the leaves are processed in the receptive cells and transported through plasmodesmata for local communication within a tissue. These signals can then enter phloem for inter-organ signaling and are transported to distantly located target cells and tissues, such as the shoot or root tips, to bring about appropriate biochemical, physiological, and/or developmental changes. Broken arrows indicate phloem (transporting food and nutrients from leaves to storage organs and growing parts of plant) and xylem (transporting water and mineral transport from roots to aerial parts of the plant). Figure adapted from Lee et al., 2011 Trends in Plant Science 16:201-210.

FIG. 2 shows the general domain structure of PDLP family members. A signal peptide (SP) is located at the N-terminus followed by two domains of unknown function (DUF26). A single pass transmembrane domain (TM) follows the region containing the DUFs and ends in a C-terminal cytosolic tail (CT). The amino acid sequence of the C-terminal tail of each PDLP family member from Arabidopsis thaliana (see the worldwide web at http://www.arabidopsis.org/) is shown (SEQ ID NOs: 14-21).

FIG. 3 shows PDLP sequences (A) an amino acid sequence comparison of PDLP1 (SEQ ID NO:1), PDLP3 (SEQ ID NO:2), and PDLP5 (SEQ ID NO:4), from A. thaliana; (B) a nucleotide sequence (SEQ ID NO:3) from A. thaliana encoding PDLP5; and (C) a sequence alignment showing several PDLP5 homologs: Arabidopsis thaliana PDLP5 (SP|Q8GUJ2|CRR2_ARATH; SEQ ID NO:4); Populus trichocarpa (Western balsam poplar) (TR|A9PGZ2|A9PGZ2_POPTR; SEQ ID NO:22); Prunus persica (Peach) (TR|M5XG08|M5XG08_PRUPE; SEQ ID NO:23); Brassica rapa subsp. pekinensis (Chinese cabbage) (TR|M4DI67|M4DI67₁₃BRARP; SEQ ID NO:24); Brassica rapa subsp. pekinensis (Chinese cabbage) (TR|M4EH70|EH70_BRARP; SEQ ID NO:25); Brassica rapa subsp. pekinensis (Chinese cabbage) (TR|R015Y2|R015Y2_9BRAS; SEQ ID NO:26); Brassica rapa subsp. pekinensis (Chinese cabbage) (TR|R0HUA4|R0HUA4_9BRAS; SEQ ID NO:27); Populus trichocarpa (TR|B9HW29|B9HW29_POPTR; SEQ ID NO:28); Arabidopsis lyrata subsp. lyrata (TR|D7KYD3|D7KYD3_ARALL; SEQ ID NO:29). The shaded region corresponds to the transmembrane domain and the boxed region corresponds to the C-terminal tail.

FIG. 4 shows the nucleotide (SEQ ID NO:5) and amino acid (SEQ ID NO:6) sequences of a PDLP5 mutant, PDLP5-m5, which contains three mutations (Cysteine→Alanine; boxed) relative to the wild type PDLP5 protein.

FIG. 5 shows fast vegetative growth of PDLP5-m5 plants. Wild type (WT) plants, plants overexpressing PDLP5 (PDLP5-OX), and plants with a severe knock down of PDLP5 (pdlp5-1) were used as comparisons. (a) Whole plants at 10, 14, and 18 days post germination (dpg). Plants were grown at 22-20° C. with ˜50% humidity and with 16/8 hours light/darkness. (b) Leaves at 3 weeks post germination. Plants were grown at 24-22° C. with ˜70% humidity and with 16/8 hours light/darkness.

FIG. 6 shows relative rosette diameter is maintained in the fast vegetative growth of PDLP5-m5 plants. Wild type (WT) plants, plants overexpressing PDLP5 (OX), and plants with a severe knock down of PDLP5 (KO) were used as comparisons. Plants overexpressing PDLP5 (OX) exhibit reduced rosette diameter. Seeds were germinated on plates, were transferred to soil after 1 week, and plants were grown at 22-20° C. with 16/8 hours light/darkness.

FIG. 7 shows early flowering of PDLP5-m5 plants. Wild type (WT) plants and plants overexpressing PDLP5 (PDLP5-OX) were used as comparisons. Plants were grown at 24-22° C. with ˜70% humidity and with 16/8 hours light/darkness.

FIG. 8 shows early flowering of PDLP5-m5 plants is associated with decreased number of rosette leaves. Wild type (WT) plants and plants overexpressing PDLP5 (PDLP5-OX) were used as comparisons.

FIG. 9 shows normal inflorescence development of PDLP5-m5 plants. Wild type (WT) plants were used as comparisons.

FIG. 10 shows PDLP5-m5 plants have normal to better seed yield when compared to wild type (WT) plants and plants with a severe knock down of PDLP5 (pdlp5-1).

FIG. 11 shows PDLP5-m5 plants have improved resistance to chilling when compared to wild type (WT) plants and plants overexpressing PDLP5 (PDLP5-OX). Plants were grown to 2 weeks post germination at 20° C. followed by 7 weeks of culture at 5° C. with 16/8 hours light/darkness.

FIG. 12 shows enhanced root branching and root growth of PDLP5-m5 plants at 10 days post germination (DPG) when compared to wild type (WT) plants. (a) PDLP5-m5 plants exhibit a 30-40% increase in root length. (b) PDLP5-m5 plants exhibit an increase in total secondary root growth relative to the wild type plants.

FIG. 13 shows plants overexpressing PDLP5 (OX), wild type (WT) plants, plants having a severe knock down of PDLP5 (pdlp5-1), and PDLP5-m5 plants (PDLP5-m5 T2-26) at the end of a 2 week water withdrawal. All plant lines were grown for 2 weeks with water prior to the 2 week water withdrawal. (a) Plants having a severe knock down of PDLP5 (pdlp5-1) and PDLP5-m5 plants bolt faster than plants overexpressing PDLP5 (OX) and wild type (WT) plants. (b) Plants overexpressing PDLP5 (OX) revive 3 days post rewatering (DPR). (c) Plants overexpressing PDLP5 (OX) fully recover and 10 days post rewatering (DPR); wild type (WT) plants are dead, and PDLP5-m5 plants continue to survive.

FIG. 14 shows that early bolting of the PDLP5-m5 plants affected plant revival after water withholding. (a) Plants overexpressing PDLP5 (OX), wild type (WT) plants, plants having a severe knock down of PDLP5 (pdlp5-1), and three different PDLP5-m5 plants (T3-4-2, T2-6, and T2-26) which were all grown a different number of days as required to reach same rosette size before water withdrawal. Plants having the same rosette size are referred to as “same day bolting samples.” (b) Same day bolting samples of plants overexpressing PDLP5 (OX), wild type (WT) plants, plants having a severe knock down of PDLP5 (pdlp5-1), and three different PDLP5-m5 plants (T3-4-2, T2-6, and T2-26) at the end of a 2 week water withdrawal. (c) Same day bolting samples of plants overexpressing PDLP5 (OX) and two different PDLP5-m5 plants (T3-4-2 and T2-26) recover fully 3 days post rewatering.

FIG. 15 shows synthesis and expression of the PDLP5-m5. (a) Schematics of PDLP5-m5, PDLP5-C, and PDLP5-2C mutants as created by overlapping PCR. (b) Transient expression of mutants in Nicotiana benthamiana leaves. PDLP5-m5 (3C-3A) expression results in more extensive viral movement, whereas PDLP5 WT protein overexpression results in a delay in viral movement. PDLP5-2C (2C-2A) or PDLP5-C (1C-1A) mutants did not show PDLP5-m5 effect.

FIG. 16 shows PDLP5 is required for normal LR emergence. (A) Lateral root development in wild type (WT) plants, plants having a severe knock down of PDLP5 (pdlp5-1), and plants overexpressing PDLP5 (PDLPOE) expressing DR5:GUS. Arrowheads indicate tertiary roots. (B) Quantification of total lateral root numbers (both emerged and unemerged secondary [2°], tertiary [3°], and quaternary [4°] roots). n≧30 per seedling set. Bars, standard deviation. Stars, significance determined by student T-test (P<0.01). (C) A diagram depicting several stages of LRP emergence. (D) Percent distribution of LRP stages, recorded at root bend at indicated time points following gravistimulation at 3 dpg. n≧20 seedlings per set.

FIG. 17 shows images and quantification of roots. (A-C) GUS-stained 11-day-old seedlings expressing DR5:GUS in wild type (WT) Col-0 plants (A), plants having a severe knock down of PDLP5 (pdlp5-1) (B), and plants overexpressing PDLP5 (PDLPOE) (C). Size bar in (A), common to (B) and (C). (D) Measurement of primary root length in 10-day-old wild type (WT) seedlings, seedlings having a severe knock down of PDLP5 (pdlp5-1), and seedlings overexpressing PDLP5 (PDLPOE). n=30 seedling per line. (E) Measurement of % emerged lateral root (LR) of total secondary roots in 10-day-old WT, pdlp5-1, and PDLP5OE seedlings. n=30 seedling per line. (F) Measurement of average secondary root length per seedling in 7-day-old WT and pdlp5-1. n=28 seedlings per line.

FIG. 18 shows spatiotemporal expression of PDLP5 in overlying cells during lateral root primordia (LRP) development. (A) Close-up of dividing pericycle cells in first stage of LRP development (arrowheads indicate first divisions). Xy, xylem; Pe, pericycle; Co, cortex; En, endodermis; Ep, epidermis. Scale bars, 10 μm. (B) GUS-staining of LRP in pre-emergence, emerging, and post-emergence stages. (C) PDLP5pro:GUS staining 2 days post shoot-removal, at various stages of LRP emergence. Stars in (B) and (C) indicate center of LRP tip. Scale bars in (B) and (C), 25 μm. (D) Close-up of GUS-stained LR initiation sites showing expression of PDLP5 in wild type (WT), shy2-2, and iaa28-1 backgrounds. Scale bars, 50 μm. (E) ChIP assay showing the upstream regions of PDLP5 containing canonical (−2341 to −2260) or core (−394 to −285) AREs are bound to ARF19. Fold enrichment is calculated as the amount of promoter fragment immunoprecipted relative to the non-immunoprecipitated input chromatin. Anti-ARF 19 immunoprecipitated DNA is normalized to input chromatin using an internal control (TUB3). Results are representative of 3 biological repeats. Bars, standard error.

FIG. 19 shows GUS-stained 7-day-old seedlings. LRI, lateral root initiation site; EZ, elongation zone; MZ, meristematic zone; RC, root cap.

FIG. 20 shows GUS-stained 7-day-old seedlings before and after shoot removal. Shoots were removed at 5 days post germination. Scale bar, 25 μm; common to all panels.

FIG. 21 shows PDLP5pro:GUS expressed in shy2-2 and iaa28-1 mutant backgrounds, showing the changes in staining pattern and intensity relative to wild type (WT) backgrounds.

FIG. 22 shows effect of PDLP5 on LAX3 expression. (A) Seedlings were gravistimulated at 3 days post germination and the LAX3::LAX3-YFP signal was monitored under a confocal microscope from 14 hours post-gravitropic induction through 36 hours post-gravitropic induction at the root bend. Images were selected to show the earliest detection time points. Arrowhead, Co cells expressing a low but detectable LAX3-YFP fluorescence. Scale bars, 50 μm. (B) Quantification of relative occurrence of LAX3-YFP signal in Co at 22 hours post-gravitropic induction (hpg) based on the data presented in Table 3.

FIG. 23 shows exogenous auxin—induction of PDLP5proGUS in roots and PD closure in leaf tissues. (A) GUS staining of napthalene acetic acid (NAA)-treated roots. Images are tiled and manually aligned. Scale bars, 50 μm. (B) Model for the possible role of PDLP5 during lateral root (LR) emergence. In the wild type (WT) plant, auxin-triggered PDLP5 limits excess diffusion of auxin through PD. This helps regulate the expression timing of genes encoding LAX3 and CWR enzymes in the cortex that positively influence LR emergence. In the plant having a severe knock-down in PDLP5 (pdlp5-1), auxin can diffuse through PD into cortical (and later, epidermal) cells earlier than it should. En, endodermis; Co, cortex; Ep, epidermis This expedites the expression of emergence-promoting genes, accelerating LR emergence.

FIG. 24 shows seven-day-old Arabidopsis seedlings expressing PDLP5pro:GUS or DR5:GUS were mock-treated with water or treated with the indicated hormones for 9 hours, followed by 3 hr GUS staining. Concentrations: salicylic acid (SA), 100 μM; JA, 50 μM; ABA, 10 μM; 6-BAP, 1 μM. n=10 per treatment for each line. Representative images are shown. Scale bar, 100 μm.

FIG. 25 shows nine-day-old Arabidopsis wild type seedlings were mock-treated with water or sprayed with the indicated hormones for 4 hours, then roots were excised, frozen, and RNA collected for RT-PCR. Relative band intensity was quantified with Image-J and standardized against ubiquitin. Concentrations: 100 μM salicylic acid (SA), 5 μM napthalene acetic acid (NAA). Three biological and two technical repeats were performed.

FIG. 26 shows PDLP5 promoter segments. (A) Upstream sequence of PDLP5 (SEQ ID NO:26) showing promoter and auxin-regulated elements. (B) Position of promoter and auxin-regulated elements including ATG, transcription start site, Y-patch, TATA box (SEQ ID NO:47), TGTC core forward sequence (SEQ ID NO:13), GACA core reverse sequence (SEQ ID NO:48), TGTCTC forward sequence (SEQ ID NO:49), and the GAGACA reverse sequence (SEQ ID NO:50).

### DETAILED DESCRIPTION

The terms “monocot” and “monocotyledonous plant” are used interchangeably herein. A monocot includes the Gramineae family. Maize, wheat and rice are exemplary monocots.

The terms “dicot” and “dicotyledonous plant” are used interchangeably herein. A dicot includes the following families: Brassicaceae, Leguminosae, and Solanaceae.

A “trait” refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic or biotic stress tolerance, greenness, stay-green, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen stress tolerance, nitrogen uptake, root lodging, root mass, harvest index, stalk lodging, plant height, ear height, ear length, salt tolerance, cold tolerance, early flowering, early seedling vigor and seedling emergence under low temperature stress. Favorable traits may be determined by observing any one of a number of agronomic characteristics and phenotypes.

Yield can be measured in many ways, including, for example, test weight, seed weight, seed number per plant, seed number per unit area (i.e. seeds, or weight of seeds, per acre), bushels per acre, tonnes per hectare, tonnes per acre, tons per acre and kilograms per hectare.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E.Westgate & K. Boote; CSSA (Crop Science Society of America)Special Publication No.29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

Plant stresses include both abiotic (non-pathogenic) stress and biotic stress. Abiotic stresses include, for example, at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., N,N′-dimethyl-4,4′-bipyridium dichloride, known by the trade name paraquat) that induce production of reactive oxygen species (ROS). Biotic stress can include exposure to pathogens or pests, such as bacterial or fungal pathogens, insects, weeds, invasive or competitive species, and the like.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions. A plant with “increased stress tolerance” can exhibit increased tolerance to one or more different stress conditions. Such plant may exhibit improved plant yield and/or fitness when exposed to abiotic or biotic plant (pathogenic) stress.

“Stress tolerance activity” of a polypeptide indicates that expression of the polypeptide in a transgenic plant confers increased stress tolerance to the transgenic plant relative to a reference or control plant.

“Drought” refers to a decrease in water availability to a plant that, especially when prolonged, can cause damage to the plant or prevent its successful growth (e.g., limiting plant growth or seed yield). “Water limiting conditions” refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions.

The terms “heat stress” and “temperature stress” are used interchangeably herein, and are defined as where ambient temperatures are hot enough for sufficient time that they cause damage to plant function or development, which might be reversible or irreversible in damage “High temperature” can be either “high air temperature” or “high soil temperature”, “high day temperature” or “high night temperature, or a combination of more than one of these.

“Modified plasmodesmal connectivity” of a plant is measured relative to a reference or control plant, and is a trait of the plant that is reflected in an altered plasmodesmal response when exposed to a plant stress condition, relative to the response seen in a comparable wild type plant, and which provides the plant with or is accompanied by at least one improved agronomic characteristic providing tolerance to a plant stress condition. Whether a genetically engineered plant exhibits modified plasmodesmal connectivity can be determined using any suitable assay. For example, suitable techniques may include the Drop-ANd-See assay method or any standard PD permeability assay as described, for example, in Lee et al. (2011 Plant Cell 23:3353-3373).

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores. The term “plant part” includes plant organs, plant tissues, plant propagules, seeds and plant cells.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes. “Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is modified from its native form in composition and/or genomic locus by deliberate human intervention.

“Regenerable plant cell” is a cell that can be regenerated into a plant and includes, but is not limited to, a callus cell, an embryogenic callus cell, a gametic cell, a meristematic cell, or a cell of an immature embryo. A regenerable plant cell may derive from an inbred maize plant.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory elements and coding sequences that are derived from different sources, or regulatory elements and coding sequences derived from the same source, but arranged in a manner different than that normally found in nature. The terms “recombinant DNA construct” and “recombinant construct” are used interchangeably herein.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory elements may include, but are not limited to, promoters, translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA). A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

### Plasmodesmata-Located Protein (PDLP) Family

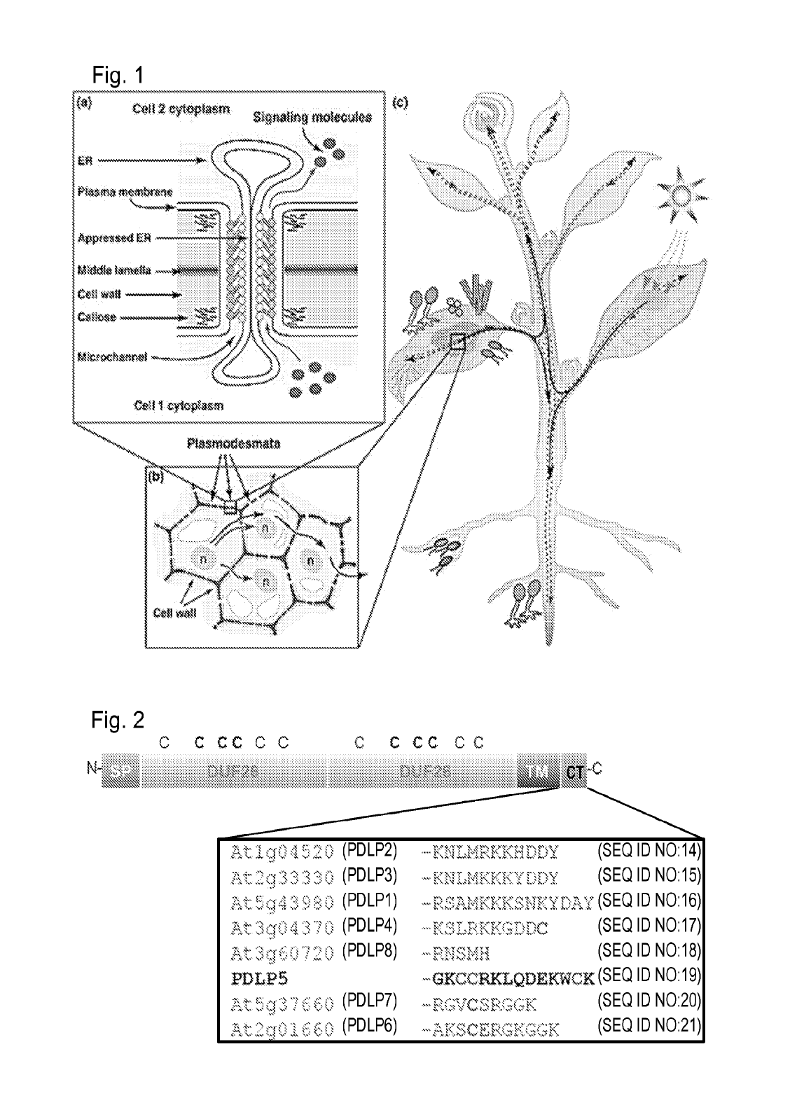

Plasmodesmata (PD) are plant-unique intercellular communication channels, which allow plant cells to share their cytoplasm and build a multicellular organism. PD serve as a signaling pathway between neighboring cells and facilitate cell-to-cell communication across the cell wall. See FIG. 1 for a schematic diagram of a plasmodesma.

A family of PD-localized proteins (PDLP) has been identified as affecting PD permeability. PDLPs typically range from 30 to 35 kD in predicted size. The general domain structure of PDLP family members is shown in in FIG. 2. A signal peptide (SP) is located at the N-terminus followed by two domains of unknown function (DUFs, more specifically, DUF26 domains). A single pass transmembrane domain (TM) follows the region containing the DUFs and ends in a cytoplasmic C-terminal tail (CT) (Thomas et al., 2008 PLoS Biol. 6:e7; Lucas et al., 2009 Trends Cell Biol. 19:495-503). The signal peptide serves to direct the protein into the secretory pathway. The transmembrane domain serves to target the protein to the PD. The C-terminal tail resides in the cytoplasm while the DUF26 domains are extracellular, being in the apoplast (Thomas et al., 2008 PLoS Biol. 6:e7). Each DUF26 domain, a plant-specific protein module, is characterized by conserved Cys residues and is found in a plant protein superfamily including Cys-rich receptor-like kinases (CRKs) and Cys-rich secretory proteins (Chen, 2001 Plant Physiol. 126:473-476).

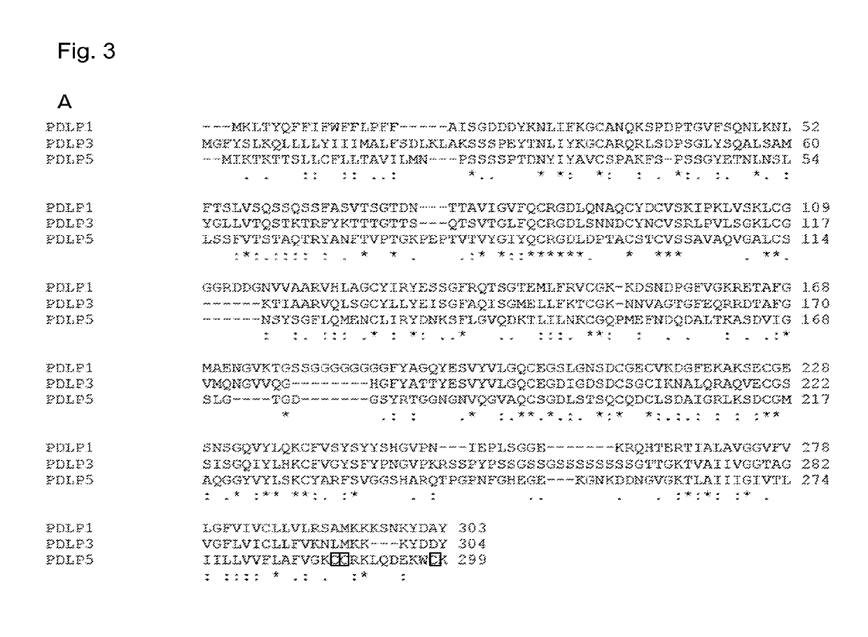

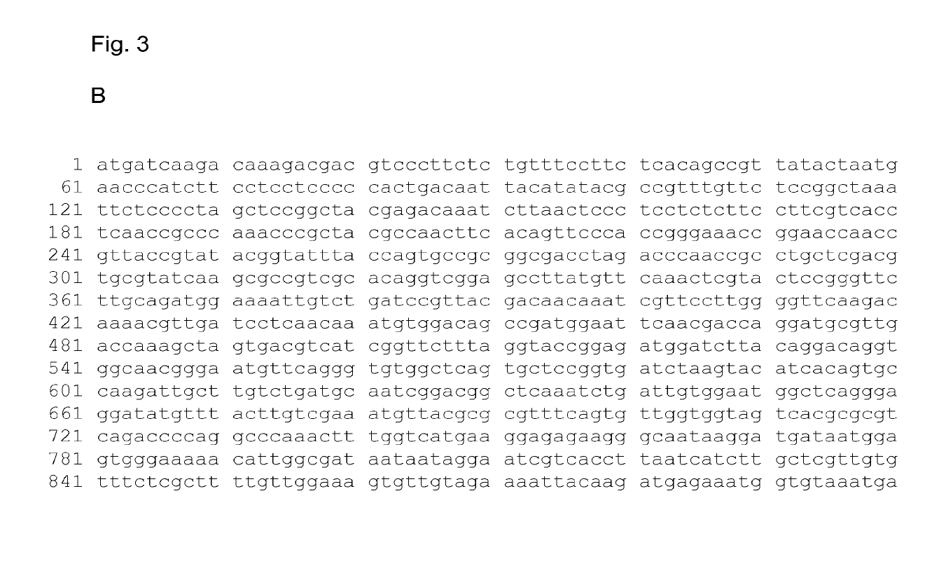

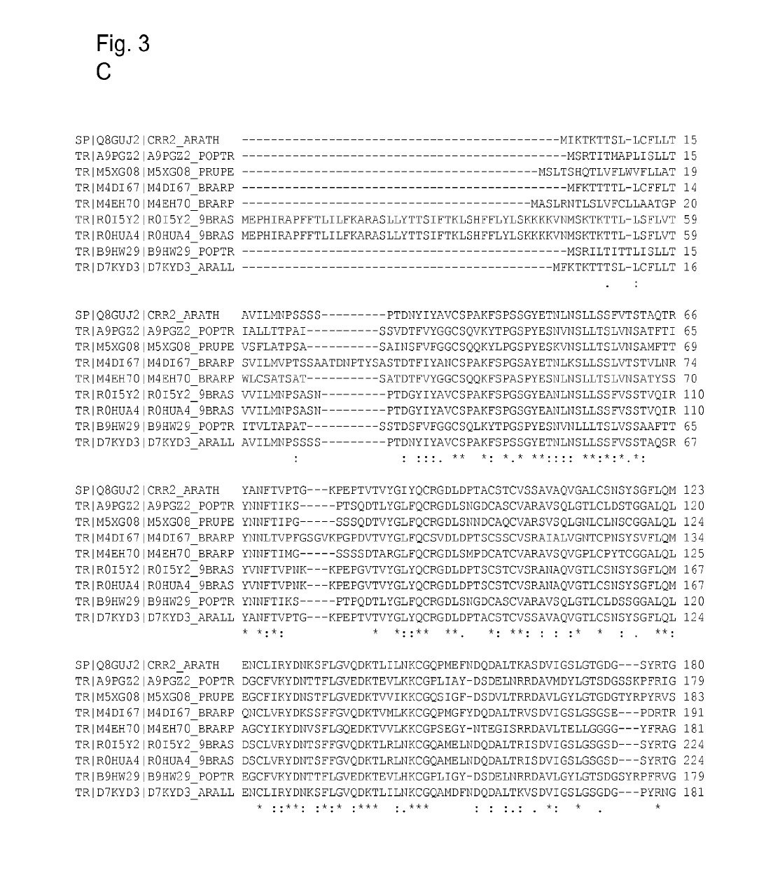

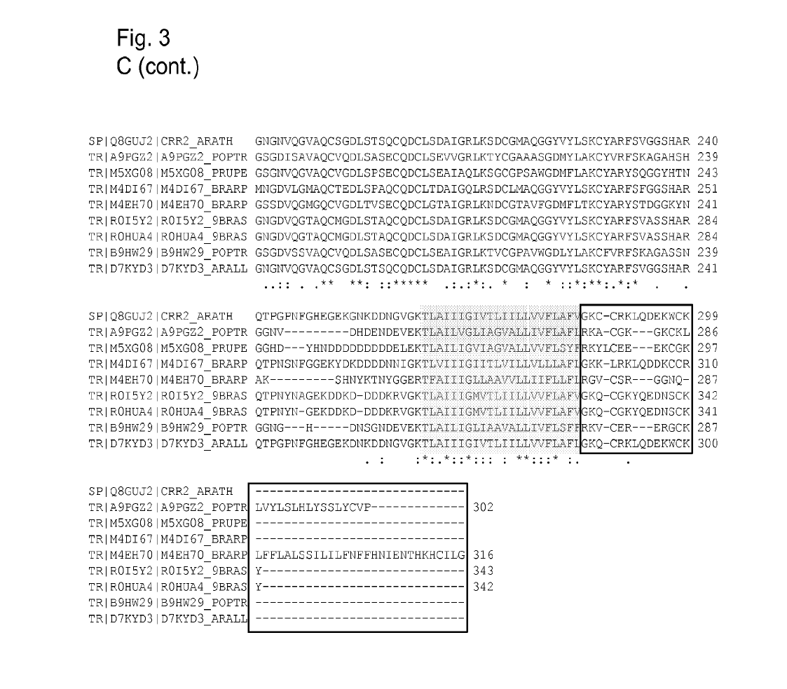

A first member of the PDLP family, PDLP1, was identified in A. thaliana (GenBank Accession No. At5g43980; SEQ ID NO:1) (Thomas et al., 2008 PLoS Biol. 6:e7). A proteomics analysis of a cell wall fraction prepared from Arabidopsis seedlings identified two additional members of the PDLP family, PDLP3 (GenBank Accession No. At2g33330; SEQ ID NO:2) and PDLP5 (GenBank Accession No. At1g70690; UniProtKB/Swiss-Prot Accession No. Q8GUJ2, available on the world wide web at uniprot.org/uniprot/Q8GUJ2; SEQ ID NO:4) (Lee et al., 2011 Plant Cell 23:3353-3373). PDLP3 and PDLP5 show ˜50 and 30% amino acid sequence identities, respectively, with PDLP1 (SEQ ID NO:1). A sequence alignment is shown in FIG. 3A.

The eight known members of the PDLP family are listed in Table 1.

TABLE 1
Members of PDLP family.

| | GenBank Locus/ | |
|---|---|---|
| Gene Name(s) | Accession | References |
| PDLP1 | AT5G43980 | 1, 2 |
| PDLP2 | AT1G04520 | 1, 2 |
| PDLP3 | AT2G33330 | 1, 2 |
| PDLP4 | AT3G04370 | 1 |
| PDLP5 (HWI1) | AT1G70690 | 1, 3 |
| PDLP6 | AT2G01660 | 1, 2 |
| PDLP7 | AT5G37660 | 1, 2 |
| PDLP8 | AT3G60720 | 1, 2 |

1 Thomas et al., 2008 PLoS Biol. 6: e7;
2 Bayer et al. 2008 Plant Signal Behav. 3: 853-855; and
3 Lee et al., 2008 Plant J. 54: 452-65.

### PDLP5 Protein

A representative PDLP5 protein was identified in A. thaliana. An A. thaliana PDLP5 amino acid sequence (SEQ ID NO:4) is shown in FIG. 3A, and a nucleotide sequence encoding an A. thaliana PDLP5 protein (SEQ ID NO:3) is shown in FIG. 3B. As noted in the UNIPROT entry on world wide web at uniprot.org/uniprot/Q8GUJ2, A. thaliana PDLP5 contains a 25 amino acid signal peptide (amino acids 1-25), a large extracellular topological domain (amino acids 26-264), a single 21 amino acid transmembrane domain (amino acids 265-285) and a short, flexible, 14 amino acid cytoplasmic domain (amino acids 286-299).

PDLP5 is believed to exist in all seed plants; FIG. 3C shows homologous PDLP5 sequences from plants as diverse as Western balsam poplar, peach, and Chinese cabbage. It should be noted that PDLP5 has also been known in Arabidopsis as HOPW1-1-INDUCED GENE1 (HWI1), and can also be referred to as cysteine-rich repeat secretory protein 2 (CRRSP2) or cysteine-rich repeat protein HWI1 (see the world wide web at uniprot.org/uniprot/Q8GUJ2).

Genetically engineered plants overexpressing PDLP5 exhibit constricted or closed PD and slower movement of nutrients and other compounds between cells, ultimately inducing spontaneous cell death. For example, movement was reduced by 70% in Arabidopsis plants overexpressing PDLP5 (Lee 2011 Plant Cell 23:3353-3373). Genetically engineered plants having a severe knock-down of PDLP5 (pdlp5-1) maintain open PD and exhibit more compounds flowing back and forth between cells. For example, movement was enhanced by 25% in Arabidopsis pdlp5-1 plants (Lee 2011 Plant Cell 23:3353-3373). It should be understood that the term engineered or genetically engineered is inclusive of the term transgenic, but also includes, for example, possessing multiple genomic copies of endogenous or homologous polynucleotides, and/or disruptions or changes in an endogenous polynucleotide, such as in a knock out or knock down strain, or altered gene expression levels and patterns or protein coding sequences, relative to a comparable wild type cell.

Surprisingly, PDLP5 polynucleotide sequences modified to delete the cytosolic tail are unable to express functional PDLP5 polypeptides, suggesting that the cytosolic tail plays an important role in PDLP5 function. A. thaliana PDLP5 differs from other members of the PDLP family in A. thaliana in that it contains three cysteine residues in its cytosolic tail (FIG. 2; compare SEQ ID NO:19 to SEQ ID NOs:14-18 and 20-21). Specifically, residues 288, 289, and 298 of the PDLP5 (SEQ ID NO:4) polypeptide sequence are cysteine residues (FIG. 3A).

### Modified PDLP5 Protein and Polynucleotide

PDLP5 is a novel target for genetic engineering so as to produce or enhance one or more favorable agronomic characteristics in a plant, including for example increased tolerance to abiotic stress, such as cold temperatures or drought, as well as biotic stress, such as pathogen infection. PDLP5-like or homologous or orthologous proteins in other plant species, such as those shown in FIG. 3C, can be identified and selected based on the finding that C-terminal cysteine residues are useful targets for modulating the function of PDLP5 in A. thaliana (see Example 1). These proteins contain at least one cysteine residue in the C-terminal cytoplasmic tail, and typically contain two or more cysteine residues in the C-terminal cytoplasmic tail. FIG. 3C shows the amino acid sequences for PDLP5 proteins from A. thaliana (SEQ ID NO:4), as well as homologs from several other organisms. The cytosolic cysteine residues in PDLP5 proteins serve to distinguish PDLP5 proteins from some other members of the PDLP family, and are target sites for modification in accordance with the present invention.

A PDLP5 protein suitable for use as a target molecule for mutation to yield a modified PDLP5 protein of the invention includes any polypeptide that is homologous to A. thaliana PDLP5 (SEQ ID NO:4), regardless of its biological source or the nomenclature assigned to it, provided it has at least one cysteine, preferably two or three or more cysteines, in the cytosolic tail region which can be mutated. An exemplary list of naturally occurring PDLP5 amino acid sequences amenable to mutation in accordance with the invention is shown in FIG. 3C.

As an example, the amino acid sequence of the cytoplasmic C-terminal tail of a representative embodiment, A. thaliana PDLP5, is GKCCRKLQDEKWCK (SEQ ID NO:19), representing amino acids 286 to 299 of the full amino acid sequence of A. thaliana PDLP5 (SEQ ID NO:4) as shown in FIG. 3A. The C-terminal sequence has three cysteine (C) residues, as positions 288, 289 and 298. These cytosolic cysteine residues are targets for modification in accordance with the present invention. It will be noted that PDLP5 proteins also contain a number of cysteine residues within their extracellular DUF26 domains (FIG. 2); however, these extracellular cysteines are not targets for modification in the present invention.

Under appropriate conditions, two cysteine residues can form a disulfide bond. Disulfide bonds can be intramolecular or intermolecular. Without intending to be bound by theory, it is suspected that modification of one, two, three or more (if present) cytosolic cysteines in the C-terminal region of a PDLP5 protein may affect PDLP5 function by interfering with the formation of one or more intramolecular and/or intermolecular bonds.

A modified PDLP5 protein is a PDLP5 protein which contains a modification of at least one cysteine residue in the C-terminal cytoplasmic tail. A modified PDLP5 protein may have a modification at one, two or all three (or more) of the PDLP5 cytosolic cysteine residues. The PDLP5 protein that is so modified can be a naturally occurring PDLP5 protein (such as a PDLP5 protein shown in FIG. 3C), including known or as yet unknown naturally occurring PDLP5 homologs and orthologs, and it likewise can be a protein that has a specified level of sequence identity or homology to a PDLP5 protein as described elsewhere herein. Advantageously, the modified PDLP5 can be introduced into a plant and expressed in a wild-type background or in a genetically modified background. The plant may, or may not, express a native form of PDLP5. Optionally, the native expression of PDLP5 may be suppressed in the plant, by any convenient means known to the art.

In some embodiments, the modified PDLP5 protein exhibits stress tolerance activity, in that expression of the modified PDLP5 protein in a genetically engineered plant confers increased stress tolerance to the transgenic plant relative to a reference or control plant. Increased stress tolerance can be, for example, increased tolerance to drought or to temperature extremes (either high or low).

In some embodiments, the modified PDLP5 protein exhibits PDLP5-m5 activity. The term “PDLP5-m5 activity” means that the protein exhibits negative gain-of-function activity similar to that exhibited by the PDLP5-m5 protein (SEQ ID NO:6), when expressed in a plant, including a plant with a wild-type PDLP5 background. Negative gain-of-function activity, including semi-dominant negative gain-of-function activity, is characterized with reference to the activity of the corresponding wild-type PDLP5 protein, in this instance, A. thaliana PDLP5.

As noted elsewhere, a polynucleotide operably encoding a modified PDLP5 protein can be introduced into the plant's genome to yield a transgenic plant of the invention. More generally, expression of the modified PDLP5 protein in a transgenic plant is expected to cause a favorable change to the plant's phenotype.

The modification that results in a modified PDLP5 protein can be a substitution of a cysteine with a different amino acid, or it can be a deletion of a cysteine. In embodiments based on a PDLP5 that natively contains more than one cytosolic cysteine residue, a combination of both substitution and deletion may optionally be used. More generally, the modification is one that results in the elimination, through whatever means, of one or more cytosolic cysteine residue from the PDLP5 protein.

Deletion of a cysteine can take the form of deletion of a single amino acid (i.e., the cysteine residue) or the deletion of cysteine residue plus one or more contiguous amino acids; in some embodiments, deletion of a cysteine can take the form of a C-terminal truncation, particularly when the cysteine is the last, the penultimate, or the antepenultimate residue. An exemplary modified PDLP5 protein has a truncation at the C-terminus of at least 2 amino acids, thereby removing a cysteine that is in the penultimate position (e.g. position 298 in A. thaliana PDLP5).

Substitution of a cysteine with a different amino acid typically takes the form of a replacement of cysteine by another single amino acid, but in some embodiments two or more amino acids can be inserted in place of the cysteine (referred to as an insertion in contrast to a substitution). It should be understood that where a substitution is performed, and insertion or two or more amino acids can likewise be used to obtain the same result and is encompassed by the invention.

An exemplary modified PDLP5 protein of the invention includes a mutation at one or more of the target cysteine residues, Cys288, Cys289 or Cys298 (as specified for A. thaliana), or their analogous positions in PDLP5 from other organisms. Modified PDLP5 proteins include PDLP5 proteins having amino acid substitutions at Cys288; at Cys289; at Cys298; at both Cys288 and Cys289; at both Cys288 and Cys298; at both Cys289 and Cys298; or at all three of Cys288, 289 and 298, as well as analogous positions in homologous PDLP5 proteins. Exemplary amino substitutions include substituting alanine in place of one or more of the cysteines, but any amino acid can be used in amino acid substitution. In one embodiment, one or more of the target cytosolic cysteines is independently substituted with an uncharged amino acid, such as alanine, isoleucine, leucine, methionine, phenylalanine, glutamine, threonine, glycine, tryptophan, proline, valine, serine, tyrosine, or asparagine. In another embodiment, one or more of the three target cysteines is independently substituted with a charged amino acid, such as glutamate, aspartate, lysine, arginine or histidine. A mixture of charged and uncharged amino acid substitutions can be employed when two more of the target cysteines are substituted. In an exemplary embodiment, one, two or three target cysteines are substituted with alanine In another embodiment, one, two or three target cysteines are independently substituted with an uncharged amino acid, for example alanine, valine, isoleucine or leucine.

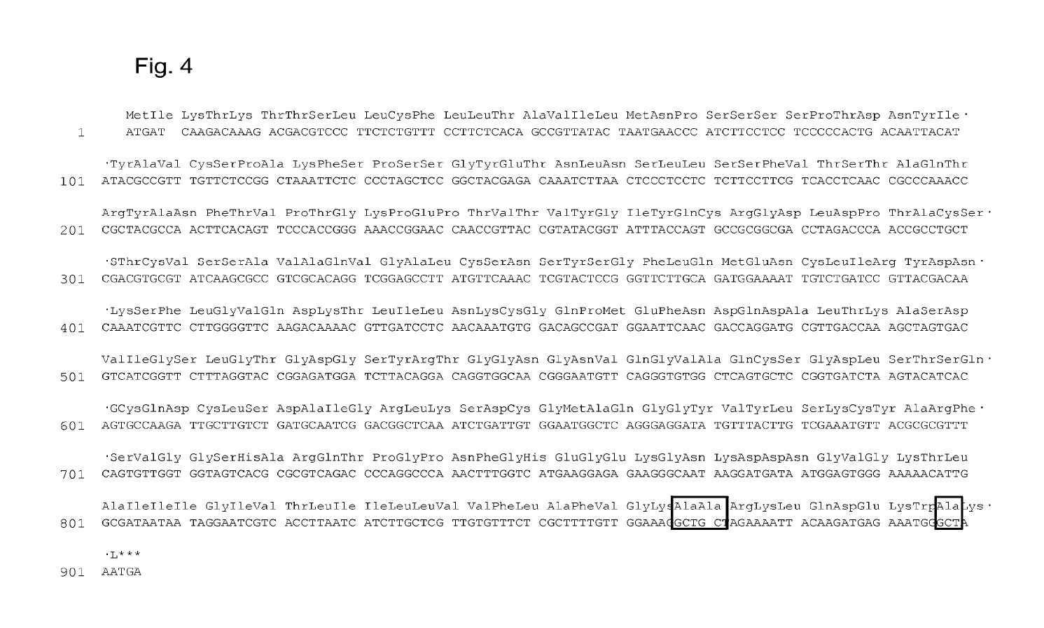

In an exemplary embodiment, one, two or three target cysteines are substituted with alanine FIG. 4 shows the nucleotide (SEQ ID NO:5) and amino acid (SEQ ID NO:6) sequences of an exemplary PDLP5 mutant, PDLP5-m5, which contains three mutations (Cysteine→Alanine; highlighted) relative to the wild-type A. thaliana PDLP5 protein. PDLP5-m5 may also be referred to herein as PDLP5m5, m5, PDLP-m5, or PDLPm5. Introducing this mutant, PDLP5-m5, into Arabidopsis as a model system enhanced the vigor of plant growth and drought resistance (see Example 1).

Optionally, a modified PDLP5 protein can further include one or more amino acid substitutions for one or more other non-cysteine residue in the cytosolic tail region or elsewhere in the protein. As used herein, the term “one or more amino acids” is intended to mean a possible number of amino acids which may be deleted, substituted, inserted and/or added by site-directed mutagenesis. For example, a modified PDLP5 protein of the invention may include an amino acid sequence having deletion, substitution, insertion and/or addition of one or more amino acids in an amino acid sequence presented in SEQ ID NO:4 or SEQ ID NO:6. Mutations at sites other than the cytosolic cysteines in the PDLP5 protein are optional, but permitted, as long as the modified PDLP5 protein retains the ability to alter PD connectivity and/or maintains semi-dominant gain-of-function activity. Such mutations are preferably conservative mutations and maintain the charge, polar or nonpolar character at the mutated site. Alterations in a nucleic acid sequence which result in the production of a chemically equivalent amino acid at a given site, but do not affect the functional properties of the encoded polypeptide, are well known in the art. A substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between charged or polar residues such as Lys-Arg, Glu-Asp or Gln-Asn replacement. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

A modified PDLP5 protein includes a PDLP5 protein having structural similarity to Arabidopsis PDLP5 (SEQ ID NO:4), in addition to a mutation at one, two or all three cysteines in the cytosolic C-terminal region. Structural similarity of two proteins can be determined by sequence alignments and/or percent identity calculations. A modified PDLP5 protein of the invention may have a specified level of sequence homology or identity to A. thaliana PDLP5 (SEQ ID NO:4), as described elsewhere herein, provided it contains an amino acid other than cysteine at position 288, or position 289, or position 298, or any combination of positions 288, 289 and 298, including all three of positions 288, 289 and 298.

In some embodiments, a modified PDLP5 protein has a cytosolic C-terminal sequence that is, or includes, an amino acid sequence selected from any of the amino acid sequences encompassed by the consensus sequence at positions 286 to 299 of (G/R)KXX(R/G/E)(K/R)(L/Y)Q(D/E)(D/E)(K/R)XX(K/R), where X represents any amino acid other than cysteine (SEQ ID NO:30). See FIG. 2.

It is understood, as those skilled in the art will appreciate, that the invention encompasses more than the specific exemplary sequences.

PDLP5 homologs and orthologs can be found by standard sequence homology comparison techniques well known to the art, followed by modifiying the naturally occurring PDLP5 as described herein to produce a host plant-derived modified PDLP5 protein having semi-dominant negative gain-of-function activity and/or exhibiting modified plasmodesmal connectivity. A recombinant DNA construct encoding the host plant-derived modified PLDP5 can be introduced into a regenerative plant cell to generate a transgenic plant cell, plant seed, other plant part or plant that is capable of expressing the modified PDLP5 protein. Alternatively, the modified PDLP5 protein can be derived from a plant that differs from the host plant, such as an A. thaliana derived modified PDLP5 protein as described herein.

In some embodiments, the modified PDLP5 protein of the invention can confer, on the plant or plant part in which it is expressed, modified plasmodesmal (cell-to-cell) connectivity. The plant or plant part may exhibit an altered PD response when exposed to a plant stress condition, relative to the response seen in a comparable wild type plant, and which provide the plant with at least one improved agronomic characteristic providing tolerance to the plant stress condition. Without intending to be bound by theory, it is suggested that genetically engineered plants and plant parts of the invention, such as seeds, may exhibit modified plasmodesmal connectivity that maintains open communication when exposed to a plant stress condition which would normally induce the PD to close. By maintaining open PD between plant cells, the flow or transport/movement of water and/or nutrients between plant cells may also be maintained. The open flow of water and/or nutrients between plant cells may activate a positive feedback loop which stimulates the plant to produce additional nutrients which are then also distributed throughout the plant via the open PD, which may be associated with increased stress tolerance conferred by a modified PDLP5 protein of the invention.

In some embodiments, the modified PDLP5 protein of the invention can confer, on the plant or plant part in which it is expressed, at least one improved agronomic characteristic.

In some embodiments, the modified PDLP5 of the invention, such as a PDLP5-m5 (SEQ ID NO:6), represents a negative gain-of-function mutation. In a wild-type background, for example, where stress-related induction of PDLP5 expression would normally cause the PD to close, concurrent stress-induced expression of the modified PDLP5 results in PD that remain somewhat open. Since plants that express the modified PDLP5 in a WT background exhibit an intermediate phenotype between the WT phenotype (PD close in reaction to stress) and a knock-down, loss-of-function phenotype (PD remain open in the presence of stress), it is referred to herein as a semi-dominant negative gain-of-function mutation. In exhibiting a semi-dominant negative gain-of-function effect in the host plant, modified PDLP5 proteins such as PDLP5-m5 are said to “subdue” the native form of PDLP5 that plants normally express, thereby preventing the native PDLP5 protein, when induced, from having its full effect in closing or constricting the PD. An exemplary modified PDLP5 protein which is a semi-dominant negative gain-of-function mutant is PDLP5-m5 (SEQ ID NO:6).

Advantageously, a semi-dominant negative gain-of-function mutation does not require deletion or inactivation of the endogenous, wild-type PDLP5 in order to confer the benefit of the altered phenotype. Therefore, the semi-dominant negative gain-of-function modified PDLP5 protein of the invention can be introduced into any plant background of choice, including a wild-type plant background, or a null background, or any genetically altered background of interest. Optionally, expression of a WT PDLP5 in the host plant can be suppressed using techniques known to the art. Exemplary gene suppression techniques are described, for example, in Allen et al., Allen et al., US Pat Pubs. 20140245497, published Aug. 28, 2014, and 20120023622, published Jan. 26, 2012. It should be further noted that a semi-dominant negative gain-of-function PDLP5 mutation does not need to be bred to and maintained as homozygous for the PDLP5 mutation.

Also included in the invention is a polynucleotide encoding a modified PDLP5 protein, which in some embodiments takes the form of a nucleotide sequence that operably encodes a modified PDLP5 protein of the invention. A protein is operably encoded if it can be expressed in a host cell or cell-free system. A modified PDLP5 protein of the invention may be encoded by a polynucleotide including deletion, substitution, insertion and/or addition of one or more nucleotides in the nucleotide sequence of SEQ ID NO:3 or SEQ ID NO:5. Nucleotide deletion, substitution, insertion and/or addition may be accomplished by site-directed mutagenesis or other techniques as mentioned herein or otherwise known to the art.

Methods of making the modified PDLP5 protein, as well as a polynucleotide encoding a modified PDLP5 protein, are also encompassed by the invention. A polynucleotide encoding modified PDLP5 protein can be generated by standard molecular biology techniques or direct gene synthesis. For example, overlapping PCR can be used to engineer Cys to Ala substitutions and create PDLP5-m5 as described in Example 1. The method for making a polynucleotide encoding PDLP-m5 protein is illustrative and can be extended to any modified PDLP5 amino acid or nucleic acid of interest.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p.6487-6500, 1982). Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein may also be a protein which is encoded by a nucleic acid comprising a nucleotide sequence comprising deletion, substitution, insertion and/or addition of one or more nucleotides in the nucleotide sequence of SEQ ID NOs:3 or 5, provided that the modified PDLP5 protein encoded by the nucleotide sequence has 0, 1 or 2 cysteines in the cytosolic C-terminal tail, preferably 0 or 1. Nucleotide deletion, substitution, insertion and/or addition may be accomplished by site-directed mutagenesis or other techniques as mentioned above.

The protein of the invention may also be a protein which is encoded by a nucleic acid comprising a nucleotide sequence hybridizable under stringent conditions with the complementary strand of the nucleotide sequence of SEQ ID NOs:3 or 5.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

In some embodiments, the modified PDLP5 protein includes an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail. Preferably, the modified PDLP5 protein has 0 or 1 cysteine in the cytosolic C-terminal tail; more preferably it has no cysteines in the cytosolic C-terminal tail. The modified PDLP5 protein may confer increased stress tolerance on the plant or plant part which expresses it. The modified PDLP5 protein may confer increased drought tolerance on the plant or plant part that expresses it.

In some embodiments, a polynucleotide includes a nucleotide sequence, wherein the nucleotide sequence is derived from SEQ ID NO:3 (A. thaliana wild-type PDLP5) or SEQ ID NO:5 (PDLP5-m5) by alteration of one or more nucleotides by at least one method selected from the group consisting of: deletion, substitution, addition and insertion.

In some embodiments, a polynucleotide includes (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:3 (A. thaliana wild-type PDLP5) or SEQ ID NO:5 (PDLP5-m5); or (ii) a full complement of the nucleic acid sequence of (i); provided that the modified PDLP5 protein encoded by the nucleic acid sequence or its complement has 0, 1 or 2 cysteines in the cytosolic C-terminal tail. Preferably, the modified PDLP5 protein encoded by the nucleic acid sequence or its complement has 0 or 1 cysteine in the cytosolic C-terminal tail; more preferably it has no cysteines in the cytosolic C-terminal tail.

In some embodiments, a polynucleotide includes (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5); or (ii) a full complement of the nucleic acid sequence of (i), wherein the full complement and the nucleic acid sequence of (i) consist of the same number of nucleotides and are 100% complementary; provided that the modified PDLP5 protein encoded by the nucleic acid sequence or its complement has 0, 1 or 2 cysteines in the cytosolic C-terminal tail. Preferably, the modified PDLP5 protein encoded by the nucleic acid sequence or its complement has 0 or 1 cysteine in the cytosolic C-terminal tail; more preferably it has no cysteines in the cytosolic C-terminal tail.

The polynucleotide may encode a modified PDLP5 protein that confers increased stress tolerance on the plant or plant part that expresses it. The polynucleotide may encode a modified PDLP5 protein that confers increased drought tolerance on the plant or plant part that expresses it.

Also included in the invention are isolated modified PDLP5 proteins, isolated polynucleotides encoding modified PDLP5 proteins, recombinant DNA constructs including polynucleotides operably encoding modified PDLP proteins, compositions (such as plants or seeds) including these recombinant DNA constructs, and methods utilizing these recombinant DNA constructs. Any of the foregoing polynucleotides may be utilized in any recombinant DNA constructs of the invention.

### Recombinant DNA Constructs

Recombinant DNA Constructs and Suppression DNA Constructs:

In one aspect, the invention includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5); or (ii) a full complement of the nucleic acid sequence of (i), provided that the modified PDLP5 protein encoded by the nucleic acid sequence or its complement has 0, 1 or 2 cysteines in the cytosolic C-terminal tail. Preferably, the modified PDLP5 protein encoded by the nucleic acid sequence or its complement has 0 or 1 cysteine in the cytosolic C-terminal tail; more preferably it has no cysteines in the cytosolic C-terminal tail.

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:3 (A. thaliana wild-type PDLP5) or SEQ ID NO:5 (PDLP5-m5); or (ii) a full complement of the nucleic acid sequence of (i); provided that the modified PDLP5 protein encoded by the nucleic acid sequence or its complement has 0, 1 or 2 cysteines in the cytosolic C-terminal tail. Preferably, the modified PDLP5 protein encoded by the nucleic acid sequence or its complement has 0 or 1 cysteine in the cytosolic C-terminal tail; more preferably it has no cysteines in the cytosolic C-terminal tail.

In another embodiment, a recombinant DNA construct comprises a polynucleotide operably linked to at least one regulatory sequence (e.g., a promoter functional in a plant), wherein said polynucleotide encodes a modified PDLP5 protein. The modified PDLP5 protein preferably has stress tolerance activity, for example drought tolerance activity . The modified PDLP5 polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, Triticum aestivum, Populus trichocarpa, Prunus persica, Brassica rapa Populus trichocarpa or Arabidopsis lyrata subsp. lyrata, for example.

### Regulatory Sequences

Typically a recombinant DNA construct includes regulatory sequences operably linked to the polynucleotide encoding the modified PDLP5.

In some embodiments, a recombinant DNA construct includes a promoter to initiate transcription of the polynucleotide encoding the modified PDLP. A“promoter” refers to a nucleic acid fragment capable of controlling transcription of another nucleic acid fragment. A “promoter functional in a plant” is a promoter capable of controlling transcription in plant cells whether or not its origin is from a plant cell. A promoter may be homologous (from the same species) or the promoter may be heterologous (from a different plant species). A promoter may be a native promoter (a single genomic fragment derived from a single gene) or a composite promoter (an engineered promoter containing a combination of elements from different origins or a combination of regulatory elements of the same origin, but not natively found together). A number of promoters can be used in recombinant DNA constructs of the invention. The promoters can be selected based on the desired outcome, and may include constitutive, cell/tissue specific, developmentally regulated, inducible, or other promoters for expression in the host plant.

Promoters that cause a gene to be expressed in most cell types at most times are commonly referred to as “constitutive promoters.” Commonly used constitutive promoters include, without limitation, cauliflower mosaic virus (CaMV) 35S promoter, plant ubiquitin promoter (Ubi), rice actin 1 promoter (Act-1), and maize alcohol dehydrogenase 1 promoter (Adh-1). High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. 1999 Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., 1985 Nature 313:810-812); rice actin (McElroy et al., 1990 Plant Cell 2:163-171); ubiquitin (Christensen et al., 1989 Plant Mol. Biol. 12:619-632, Christensen et al., 1992 Plant Mol. Biol. 18:675-689); pEMU (Last et al., 1991 Theor. Appl. Genet. 81:581-588); MAS (Velten et al., 1984 EMBO J. 3:2723-2730); promoter (U.S. Pat. No. 5,659,026), the constitutive synthetic core promoter SCP1 (International Publication No. WO 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and U.S. Pat. No. 6,177,611.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. “Tissue-specific promoter” and “tissue-preferred promoter” are used interchangeably, and refer to a promoter that is expressed predominantly but not necessarily exclusively in one tissue or organ, but that may also be expressed in one specific cell. As used herein “tissue-specific” also includes cell-specific promoters. “Developmentally regulated promoter” refers to a promoter whose activity is determined by developmental events. Any identifiable promoter may be used in the methods of the invention which causes the desired temporal and spatial expression.

Exemplary tissue specific promoters include promoters that function in the epidermal layer (e.g., Arabidopsis ML1 promoter), phloem-specific promoters such as AtSUT2 promoter, green tissue-specific promoters such as RuBisCo small subunit promoter, and lateral root-primordia and overlying cell-specific promoters.

Promoters which are seed or embryo-specific and may be useful include soybean Kunitz trypsin inhibitor (Kti3, Jofuku and Goldberg, 1989 Plant Cell 1:1079-1093), patatin (potato tubers) (Rocha-Sosa et al. 1989 EMBO J. 8:23-29), convicilin, vicilin, and legumin (pea cotyledons) (Rerie et al. 1991 Mol. Gen. Genet. 259:149-157; Newbigin et al. 1990 Planta 180:461-470; Higgins et al. 1988 Plant. Mol. Biol. 11:683-695), zein (maize endosperm) (Schemthaner et al. 1988 EMBO J. 7:1249-1255), phaseolin (bean cotyledon) (Segupta-Gopalan et al. 1985 Proc. Natl. Acad. Sci. U.S.A. 82:3320-3324), phytohemagglutinin (bean cotyledon) (Voelker et al. 1987 EMBO J. 6:3571-3577), B-conglycinin and glycinin (soybean cotyledon) (Chen et al. 1988 EMBO J. 7:297-302), glutelin (rice endosperm), hordein (barley endosperm) (Marris et al. 1988 Plant Mol. Biol. 10:359-366), glutenin and gliadin (wheat endosperm) (Colot et al. 1987 EMBO J. 6:3559-3564), and sporamin (sweet potato tuberous root) (Hattori et al. 1990 Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., 1989 Bio/Technology 7:L929-932), bean lectin and bean beta-phaseolin promoters to express luciferase (Riggs et al., 1989 Plant Sci. 63:47-57), and wheat glutenin promoters to express chloramphenicol acetyl transferase (Colot et al., 1987 EMBO J 6:3559-3564).

Additional promoters for regulating the expression of the nucleotide sequences of the invention in plants are stalk-specific promoters. Such stalk-specific promoters include the alfalfa S2A promoter (GenBank Accession No. EF030816; Abrahams et al., 1995 Plant Mol. Biol. 27:513-52) and S2B promoter (GenBank Accession No. EF030817) and the like.

Inducible promoters selectively express an operably linked DNA sequence in response to the presence of an endogenous or exogenous stimulus, for example by chemical compounds (chemical inducers) or in response to environmental, hormonal, chemical, and/or developmental signals (physical inducers). Inducible or regulated promoters include, for example, promoters regulated by light, heat, stress, flooding or drought, phytohormones, wounding, or chemicals such as ethanol, jasmonate, salicylic acid, or safeners.

Examples of suitable chemically inducible promoters include, without limitation, Es (which stimulates expression in response to the non-plant steroid estradiol). Examples of suitable physically inducible promoters include, without limitation, heat-inducible promoter barley Hvhsp17 (Freeman et al., 2011 Plant Biotechnology Journal 9:788-796), and a stress-induced promoter complex ABA-inducible promoter complex (Vendruscolo et al., 2007 Journal of Plant Physiology 164:1367-1376). Additional inducible promoters are known in the art (see, for example, US 2001/047525 A1, US 5837848A, US 5023179A, US 7888556B2, US 2012/0210463A1, and US 6518483B1). In one embodiment, the polynucleotide encoding the modified PDLP is under the control of a physically inducible promoter which stimulates expression in response to exposure to plant stress.

Additional promoters include the following: 1) the stress-inducible RD29A promoter (Kasuga et al. 1999 Nature Biotechnol. 17:287-91); 2) the barley promoter, B22E; expression of B22E is specific to the pedicel in developing maize kernels (Klemsdal et al., 1991 Mol. Gen. Genet. 228:9-16); and 3) maize promoter, Zag2 (Schmidt et al., 1993 Plant Cell 5:729-737; Theissen et al. 1995 Gene 156:155-166; NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published July 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664).

Recombinant DNA constructs of the invention may also include other regulatory sequences, including but not limited to, translation leader sequences, introns, and polyadenylation recognition sequences. In another embodiment of the invention, a recombinant DNA construct of the invention further includes an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

In some embodiments, a recombinant DNA construct also includes a reporter gene. A reporter gene encodes a protein with an easily detectable phenotype that not only allows one to confirm expression of the expressed protein, but also enables the analysis and/or observation of the localization of the expressed protein. The reporter gene may be attached to the same regulatory sequence(s) of polynucleotide encoding the modified PDLP5, or the reporter gene may be under the control of an independent regulatory sequence(s). Suitable reporter genes include, without limitation, beta-glucuronidase (GUS), luciferase, and fluorescent proteins.

In some embodiments, a recombinant DNA construct may also include a selectable marker. A selectable marker encodes a protein that confers a transformed plant with trait that allows one to distinguish between transformed from non-transformed plants. Typically, a selectable marker is under the control of an independent, constitutive promoter. In some embodiments, a selectable marker encodes a protein that enables a transformed plant to survive in the presence of a normally toxic compound. The protein encoded by selectable marker genes generally renders these selective agents harmless to the transgenic plant. The most often used selective agents include, for example, antibiotics (such as kanamycin and hygromycin), antimetabolites, and herbicides (such as glufosinate).

Any plant can be selected for the identification of regulatory sequences and polynucleotides encoding a modified PDLP5 to be used in recombinant DNA constructs and other compositions (e.g. transgenic plants, seeds and cells) and methods of the invention. Examples of suitable plants for the isolation of genes and regulatory sequences and for compositions and methods of the invention would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini

Presence of the transgene and the determination of whether a modified PDLP5 is expressed can easily be made by a person of skill in the art using any basic in vitro or in vivo assays. Methods based on foreign DNA detection include, without limitation, Southern blot analysis, and polymerase chain reaction (PCR) assay. Methods based on RNA detection include, without limitation, northern blot analysis, reverse-transcriptase PCR, and in situ hybridization. Methods based on protein detection include, without limitation, enzyme linked immunosorbent assays (ELISA), western blot analysis, lateral flow strip assay, and immunohistochemistry. Common methods for measuring the amount of the protein may include, without limitation, chromatographic techniques such as size exclusion chromatography, separation based on charge or hydrophobicity, ion exchange chromatography, affinity chromatography, or liquid chromatography.

The invention further includes a genetically engineered plant that includes a nucleotide sequence encoding the modified PDLP5 protein of the invention and optionally the modified PDLP5 protein. The plant may be a monocot or a dicot. The modified PDLP5 protein can be expressed in the plant. Expression of the modified PDLP5 protein may be constitutive or regulated, as further described elsewhere herein. Advantageously, a plant that expresses a modified PDLP5 of the invention exhibits one or more favorable agronomic characteristics, such as resistance or tolerance to drought, or to infection by a pathogen, such as a microbial or fungal pathogen.

Also included is a genetically engineered plant part, including a plant seed, includes a nucleotide sequence encoding the modified PDLP5 protein of the invention. Optionally the modified PDLP5 protein is expressed in the plant part. Expression may be constitutive or regulated, as further described elsewhere herein.

Methods of introducing a nucleotide sequence encoding the modified PDLP5 protein of the invention into a plant or plant part, such as a seed, to yield a genetically engineered plant or plant part, such as a seed, are also included in the invention, as are methods of using the genetically engineered plant or plant part, such as a seed, which may include planting the genetically engineered plant seed and/or growing or harvesting the genetically engineered plant.

In some embodiments, the genetically engineered plants or plant parts, including seeds, may have increased stress tolerance, such as drought tolerance.

In some embodiments, the genetically engineered plant is a crop plant. A crop plant is any plant or plant product grown and harvested extensively for subsistence. Crop plants may include food crops (for human consumption) including but not limited to field crops such as corn (field, sweet, popcorn), hops, jojoba, peanuts, rice, safflower, small grains (barley, oats, rye, wheat, etc.), or leguminous plants (beans, lentils, peas, soybeans); vegetable crops such as artichokes, kohlrabi, arugula, leeks, asparagus, lettuce (e.g., head, leaf, romaine), bok Choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, chinese cabbage, peppers, collards, potatoes, cucumbers, pumpkins, cucurbits, radishes, dry bulb onions, rutabaga, eggplant, salsify, escarole, shallots, endive, garlic, spinach, green onions, squash, greens, beet (sugar beet and fodder beet), sweet potatoes, swiss chard, horseradish, tomatoes, kale, turnips, and spices; and fruit and vine crops such as apples, apricots, cherries, nectarines, peaches, pears, plums, prunes, quince almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, and lychee. Crop plants may also include feed crops (for livestock consumption) including but not limited to, corn, soy, oats, and alfalfa. Crop plants may also include fibre crops for cordage and textiles (e.g., cotton, flax, hemp, jute); oil crops for consumption or industrial uses (e.g., rape, mustard, poppy, olives, sunflowers, coconut, castor oil plants, cocoa beans, groundnuts); energy crops used to make biofuels such as bioethanol (e.g., switchgrass and giant miscanthus) or biodiesel (e.g., rapeseed and soybean); and industrial crops for various personal and industrial uses (e.g., coffee, sugarcane, tea, tobacco and natural rubber plants). In some embodiments, a crop plant is a food crop. In some embodiments, a food crop is corn.

In other embodiments, the genetically engineered plant is a medicinal plant or herb, such as blackberry, black cohosh, calendula, cayenne, german, cleavers, comfrey, crampbark, dandelion, echinacea (purple coneflower), elder, fennel, ginger, ginseng, goldenseal, gumweed, hawthorn, marshmallow, mugwort, mullein, nettle, peppermint, pipsissewa, plantain, St. John's Wort, skullcap, turmeric, valerian, vitex, willow bark, yarrow, or yellow hock.

In other embodiments, the genetically engineered plant is an ornamental plant. Ornamental plants are plants that are grown for decorative purposes in gardens and landscape design projects, as houseplants, for cut flowers and specimen display. Ornamental plants are plants which are grown for display purposes, rather than functional ones; although some may be both functional and ornamental. For example, food crops that may also be used as ornamental plants include, without limitation, strawberry, rhubarb, loose-leaf lettuce, blueberry, and citrus. Ornamental plants come in a range of shapes, sizes and colors suitable to a broad array of climates, landscapes, and gardening needs. Ornamental plants may be annuals or perennials. Ornamental plants may include garden plants for the display of aesthetic features (such as flowers, leaves, scent, overall foliage texture, fruit, stem and bark, and aesthetic form) including but not limited to geranium, morning glory, marigold, or hydrangea. Ornamental plants may include ornamental trees, used as part of a garden or landscape setting including but not limited to eastern redbuds, kousa dogwood, lilac, Japanese maple, magnolia, or crabapple.

Photosynthesis is the process in plant metabolism that converts carbon dioxide and water into oxygen and glucose, and is well understood in the art. Photorespiration may also be referred to as C2 photosynthesis. Alternative carbon fixation pathways include C3 carbon fixation, C4 carbon fixation, and CAM photosynthesis. Non-limiting examples of C3 plants include rice and barley. Non-limiting examples of C4 plants include Poaceae grass species and the food crops maize, sugar cane, millet, and sorghum. Non-limiting examples of CAM plants include epiphytes (e.g., orchids, bromeliads), succulent xerophytes (e.g., cacti, cactoid Euphorbias), hemiepiphytes (e.g., Clusia); lithophytes (e.g., Sedum, Sempervivum); terrestrial bromeliads; and wetland plants (e.g., Isoetes, Crassula (Tillaea), Lobelia). In addition, studies are currently underway to convert C3 plants into C4 plants (Von Caemmerer et al., 2012 “The Development of C4 Rice: Current Progress and Future Challenges,” Science 336(6089):1671-1672). Thus, in some embodiments, a C4 plant may be a plant that has been converted from a C3 plant.

Maize is an exemplary C4 crop plant that can be agronomically enhanced by the introduction of a recombinant DNA construct of the invention. Maize is generally cold-intolerant and its root system is generally shallow, so the plant is dependent on soil moisture. Maize is most sensitive to drought at the time of silk emergence, when the flowers are ready for pollination. The C4 leaf anatomy relies on PD to transfer photosynthates between the bundle sheath cells and mesophyll cells. Cold induces plasmodesmal frequency changes at the interfaces between mesophyll, bundle sheath, and parenchyma cells, and during drought, sucrose transport from leaf into the ovules is blocked. Maize (and other C4 plants) may be particularly amenable to the effects of a modified PDLP5 of the invention, which may enhance plasmodesmal connectivity. Expression of a modified PDLP5 protein of the invention (from any source, for example, a PDLP5-m5 protein) in a maize plant can improve cold tolerance, drought tolerance, and/or other favorable agronomic attributes. In some embodiments, expression is under the control of a constitutive promoter. In some embodiments, expression is under the control of a cell- or tissue-specific promoter. Exemplary tissue- or cell-specific expression includes, without limitation, expression in the leaf, root, reproductive organs such as the silk or ear, mesophyll and bundle sheath, and/or endodermis. In some embodiments, expression is under the control of a temperature-inducible promoter, such as a heat-inducible promoter. Optionally, a PDLP5 homolog can be identified in maize through analysis of the maize leaf PD-cell wall proteome, and the maize PDLP5 homolog can be genetically engineered as described herein to yield a maize-derived modified PDLP5 protein having semi-dominant negative gain-of-function activity. The modified protein, when expressed in a plant (maize or other plant) can modify plasmodesmal connectivity in the plant. The maize-derived modified PDLP5 protein can be introduced into a regenerative maize cell to yield a transgenic cell, seed, plant part, or plant as described herein. The modified PDLP5 protein can be expressed in any desired maize background. In some embodiments, the maize background includes expression of the wild-type PDLP. In other embodiments, expression of the WT PDLP5 protein in the maize plant can be suppressed using techniques known to the art. Exemplary gene suppression techniques are described, for example, in Allen et al., US Pat Pubs. 20140245497, published Aug. 28, 2014, and 20120023622, published Jan. 26, 2012.

It should be noted that Arabidopsis thaliana is commonly used as a plant model system to demonstrate proof-of-principle. It is well understood that, although Arabidopsis is not a crop plant, successful demonstration of a genetic modification exhibiting a desirable phenotype in Arabidopsis is typically also successful in other plants, including crop plants such as maize and ornamental plants.

### Compositions

A composition of the invention includes a genetically engineered microorganism, cell, plant, and seed including the recombinant DNA construct. The genetically engineered microorganism, cell, plant, and seed may be transgenic. The cell may be eukaryotic, e.g., a yeast, insect or plant cell, or prokaryotic, e.g., a bacterial cell.

A composition of the invention is a plant includes in its genome any of the recombinant DNA constructs of the invention (such as any of the constructs discussed above). Compositions also include any progeny of the plant, and any seed obtained from the plant or its progeny, wherein the progeny or seed includes within its genome the recombinant DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under water limiting conditions), or used in a breeding program to produce hybrid seed, which can be grown to produce plants that would exhibit such an altered agronomic characteristic. The seeds may be maize seeds.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant. Particular embodiments include but are not limited to the following:

A plant (for example, a maize, rice or soybean plant) comprising in its genome a recombinant DNA construct comprising a polynucleotide operably linked to at least one regulatory sequence, wherein said polynucleotide encodes a modified PDLP5 protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail. Preferably, the modified PDLP5 protein has 0 or 1 cysteine in the cytosolic C-terminal tail; more preferably it has no cysteines in the cytosolic C-terminal tail. Preferably the plant exhibits increased drought tolerance when compared to a control plant that does not contain the recombinant DNA construct. The plant may further exhibit an alteration of at least one agronomic characteristic when compared to the control plant.

A plant (for example, a maize, rice or soybean plant) comprising in its genome a recombinant DNA construct comprising a polynucleotide operably linked to at least one regulatory sequence, wherein said polynucleotide encodes a modified PDLP5 protein, and wherein said plant exhibits increased drought tolerance when compared to a control plant not comprising said recombinant DNA construct. The plant may further exhibit an alteration of at least one agronomic characteristic when compared to the control plant.

A plant (for example, a maize, rice or soybean plant) comprising in its genome a recombinant DNA construct comprising a polynucleotide operably linked to at least one regulatory sequence, wherein said polynucleotide encodes a modified PDLP5 protein, and wherein said plant exhibits an alteration of at least one agronomic characteristic when compared to a control plant not comprising said recombinant DNA construct.

A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NOs:3 or 5; or (b) derived from SEQ ID NOs:3 or 5 by alteration of one or more nucleotides by at least one method selected from the group consisting of: deletion, substitution, addition and insertion; and wherein said plant exhibits increased tolerance to drought stress, when compared to a control plant not comprising said recombinant DNA construct. The plant may further exhibit an alteration of at least one agronomic characteristic when compared to the control plant.

A plant (for example, a maize, rice or soybean plant) comprising in its genome a recombinant DNA construct comprising a polynucleotide operably linked to at least one regulatory element, wherein said polynucleotide encodes a modified PDLP5 protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail. Preferably, the modified PDLP5 protein has 0 or 1 cysteine in the cytosolic C-terminal tail; more preferably it has no cysteines in the cytosolic C-terminal tail. Preferably, the plant exhibits an alteration of at least one agronomic characteristic when compared to a control plant not comprising said recombinant DNA construct.

A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NOs:3 or 5; or (b) derived from SEQ ID NOs:3 or 5 by alteration of one or more nucleotides by at least one method selected from the group consisting of: deletion, substitution, addition and insertion; and wherein said plant exhibits an alteration of at least one agronomic characteristic when compared to a control plant not comprising said recombinant DNA construct.

A plant (for example, a maize, rice or soybean plant) comprising in its genome a polynucleotide (optionally an endogenous polynucleotide) operably linked to at least one regulatory element, wherein said polynucleotide encodes a modified PDLP5 protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail; preferably 0 or 1 cysteine in the cytosolic C-terminal tail; more preferably no cysteines in the cytosolic C-terminal tail; and wherein said plant exhibits at least one trait selected from the group consisting of: increased drought tolerance, increased yield, increased biomass, increased cold tolerance, early flowering and altered root architecture, when compared to a control plant not comprising the recombinant regulatory element. The at least one regulatory element may comprise an enhancer sequence or a multimer of identical or different enhancer sequences. The at least one regulatory element may comprise one, two, three or four copies of the CaMV 35S enhancer.

Any progeny of the plants in the embodiments described herein, any seeds of the plants in the embodiments described herein, any seeds of progeny of the plants in embodiments described herein, and cells from any of the above plants in embodiments described herein and progeny thereof.

In any of the embodiments described herein, the recombinant DNA construct (or suppression DNA construct) may comprise at least a promoter functional in a plant as a regulatory sequence.

In any of the embodiments of the invention, the alteration of at least one measurable agronomic characteristic can be in the form of either an increase or decrease in that characteristic.

In any of the embodiments described herein, the at least one agronomic characteristic may be selected from the group consisting of: abiotic stress tolerance, greenness, stay-green, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen stress tolerance, nitrogen uptake, root lodging, root mass, harvest index, stalk lodging, plant height, ear height, ear length, salt tolerance, cold tolerance, early flowering, early seedling vigor and seedling emergence under low temperature stress. For example, the alteration of at least one agronomic characteristic may be an increase, e.g., in drought tolerance, yield, stay-green or biomass (or any combination thereof), or a decrease, e.g., in root lodging.

In any of the embodiments described herein, the plant may exhibit the alteration of at least one agronomic characteristic when compared, under water limiting conditions, to a control plant not comprising said recombinant DNA construct (or said suppression DNA construct). In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

The disclosure includes a method for transforming a cell (or microorganism) comprising transforming a cell (or microorganism) with any of the isolated polynucleotides or recombinant DNA constructs of the invention. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes. Examples of plant cells that can be transformed according to the invention include, without limitation, a regenerable plant cell, such as a stem cell or a meristemic cell, a differentiated plant cell such as a leaf cell or a root cell, or a plant cell that has been hormonally treated to de-differentiate it into, for example, a callus cell.

Transformation may be stable or transient. In some embodiments, a genetic modification resulting from transformation can be transferred to a different genetic background using plant breeding techniques.

The disclosure also a includes method for producing a transgenic plant comprising transforming a plant cell with any of the isolated polynucleotides or recombinant DNA constructs of the invention and regenerating a transgenic plant from the transformed plant cell. The invention is also directed to the transgenic plant produced by this method, and transgenic seed obtained from this transgenic plant. The transgenic plant obtained by this method may be used in other methods of the invention.

The invention also includes a method for isolating a polypeptide of the invention from a cell or culture medium of the cell, wherein the cell comprises a recombinant DNA construct comprising a polynucleotide of the invention operably linked to at least one regulatory sequence, and wherein the transformed host cell is grown under conditions that are suitable for expression of the recombinant DNA construct.

The invention further includes methods for increasing drought tolerance in a plant, methods for evaluating drought tolerance in a plant, methods for increasing pathogen tolerance in a plant, methods for altering an agronomic characteristic in a plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Introducing a Modified PDLP5 Protein into a Plant

The introduction of a modified PDLP5 protein into a plant involves expression of one or more polynucleotides encoding a modified PDLP5 protein as described herein. The genetically engineered plant described herein is a transgenic plant.

Also included is the use of a recombinant DNA construct for producing a plant that exhibits at least one trait selected from the group consisting of: increased drought tolerance, increased yield, increased biomass, increased cold tolerance, early flowering and altered root architecture, when compared to a control plant not including said recombinant DNA construct, wherein the recombinant DNA construct includes a polynucleotide operably linked to at least one regulatory element, wherein the polynucleotide encodes a modified PDLP5 protein having an amino acid sequence of at least 70%, 75%, 80%, 85%, 90%, 91%, 92%, 93%, 94%, 95%, 96%, 97%, 98%, 99% or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines. The polypeptide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

In preferred embodiments, the recombinant polynucleotide is introduced in a plant and stably transformed.

As will be appreciated by a person of skill in the art, expression of a modified PDLP protein can be achieved through a number of molecular biology techniques. For example, the introduction of recombinant DNA constructs encoding a modified PDLP protein into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276. More common methods of engineering transgenic plants are known in the art and include, without limitation, molecular techniques such as floral dipping (also referred to as the Agrobacterium method), and mechanical techniques such as bombardment (also referred to as the biolistic method or gene gun delivery).

In some embodiments, a polynucleotide encoding a modified PDLP protein is introduced into the genetically engineered plant using the floral dipping method. A floral dipping protocol is described in Clough and Bent (1998 Plant J. 16:735-743). Agrobacterium tumefaciens is a naturally occurring organism that is capable of inter-kingdom gene transfer and can therefore be adapted to transform a plant. Briefly, the Agrobacterium method uses A. tumefaciens, to introduce a transfer DNA, or T-DNA, into the host's nuclear DNA. A polynucleotide encoding a modified PDLP protein can be introduced into an A. tumefacienscell using a vector and standard molecular biology techniques. The vector can be any molecule that may be used as a vehicle to transfer genetic material into a cell for replication or expression. Examples of vectors include plasmids, viral vectors, cosmids, and artificial chromosomes, without limitation. A recombinant DNA construct designed for transformation (i.e., a “transformation cassette”) may include one or more copies of a polynucleotide encoding a modified PDLP5 protein. The recombinant DNA construct may be circular or linear. A recombinant DNA construct be inserted into the Agrobacteria by any means. Methods of inserting a transformation cassette into a bacterium are well known in the art and include, without limitation, transfection, electroporation or particle bombardment. The Agrobacterium containing the transformation cassette may then be used to infect a plant and integrates the transformation cassette into the plant genome.

In the floral dipping method, plants are grown to a specific life cycle point, dipped into an inoculation medium containing A. tumefacienscarrying the transformation cassette, and allowed to grow to maturity (Clough and Bent, 1998 Plant J. 16:735-743). In some embodiments, the Agrobacterium method is applied to flowering plants. In order to grow new plants with the transgene, it is necessary to insert the transgene into the sex cells of the plants.

An exemplary transformation protocol is as described in Bott (“Generation and Screening of T-DNA Insertion Mutants that Alter Localization of PDLP5,” Senior thesis submitted to fulfill requirements for a Degree with Distinction from the University of Delaware, Spring 2012, available from the University of Delaware Library through the world wide web at udspace.udel.edu/handle/19716/11326). Briefly, plants are grown to the desired stage, a suitable medium is inoculated with A. tumefaciens carrying the transformation cassette, and plants are dipped into the inoculated medium. In one embodiment, 4-week-old Arabidopsis thaliana, Col-0 plants are dipped into a solution containing Agrobacteria strain GV3101 transformed with a pGWB-35S:PDLP5-m5 recombinant construct to produce transgenic plants expressing PDLP5-m5. Transgenic T1 plants were selected on basta⁻ plates and homozygous T2 lines were identified by segregation test on T3 plants (Example 1).

In some embodiments, the genetically engineered plant may be further engineered to introduce additional traits into the plant. The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes. In such an embodiment, “genetically engineered plant” also includes reference to plants which includes more than one heterologous polynucleotide within their genome. Each heterologous polynucleotide may confer a different trait to the transgenic plant.

The method for transforming a cell (or microorganism) can include transforming a cell (or microorganism) with any of the isolated polynucleotides or recombinant DNA constructs of the invention. The cell (or microorganism) transformed by this method is also included in the invention. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

The method for producing a transgenic plant can include transforming a plant cell with any of the isolated polynucleotides or recombinant DNA constructs described herein and regenerating a transgenic plant from the transformed plant cell. The invention is also directed to the transgenic plant produced by this method, and transgenic seed obtained from this transgenic plant. The transgenic plant obtained by this method may be used in other methods of the present invention.

The invention also provides for a method for producing a plant that exhibits at least one trait selected from the group consisting of increased drought tolerance, increased yield, increased biomass, increased cold tolerance, early flowering and altered root architecture, includes growing a plant from a seed including a recombinant DNA construct, wherein the recombinant DNA construct includes a polynucleotide operably linked to at least one regulatory element, wherein the polynucleotide encodes a modified PDLPS protein having an amino acid sequence of at least 70%, 75%, 80%, 85%, 90%, 91%, 92%, 93%, 94%, 95%, 96%, 97%, 98%, 99% or 100% sequence identity, based on the Clustal V or Clustal W method of alignment, when compared to SEQ ID NO:4 (A. thaliana wild-type PDLPS) or SEQ ID NO:6 (PDLPS-m5), provided that the modified PDLP5 protein has 0, 1 or 2 cysteines in the cytosolic C-terminal tail, preferably 0 or 1 cysteine; more preferably no cysteines, wherein the plant exhibits at least one trait selected from the group consisting of: increased drought tolerance, increased yield, increased biomass, increased cold tolerance, early flowering and altered root architecture, when compared to a control plant not including the recombinant DNA construct. The modified PDLP5 protein may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

One may evaluate altered root architecture in a controlled environment (e.g., greenhouse) or in field testing. The evaluation may be under limiting or non-limiting water conditions. The evaluation may be under simulated or naturally-occurring low or high nitrogen conditions. The altered root architecture may be an increase in root mass. The increase in root mass may be at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45% or 50%, when compared to a control plant not including the recombinant DNA construct.

Also provided are methods of using plants having increased stress tolerance. Plants described herein have increased stress tolerance activity. Methods of plants having increased stress tolerance include growing the plant under exposure to abiotic and biotic plant stress. Methods of using plants having increased stress tolerance also include providing plants having at least one improved agronomic characteristic when exposed to plant stress. In some embodiments, the methods include providing at least one improved agronomic characteristic when exposed to drought conditions. In other embodiments the methods include providing at least one improved agronomic characteristic when exposed to plant stress when exposed to pathogens.

### Drought Tolerant Plants

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the invention in which a control plant is utilized (e.g., compositions or methods as described herein). In the case of a plant comprising a recombinant DNA construct, for example, the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence

Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Plants having increased stress tolerance may be tolerant to abiotic plant stress. Abiotic stresses include at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS). Provided herein are methods for increasing tolerance to abiotic plant stress. In some embodiments, the methods for increasing tolerance to an abiotic plant stress include providing a plant having modified plasmodesmal connectivity, and growing the plant under exposure to the abiotic plant disorder. In other embodiments, the methods for increasing tolerance to an abiotic plant disorder include providing a plant seed having modified plasmodesmal connectivity, and growing the plant seed under exposure to the abiotic plant stress.

In some embodiments, increasing tolerance to abiotic plant stress includes increasing drought tolerance. Plants have many natural adaptations for drought conditions, including adaptations of the stomata to reduce water loss, water storage in succulent above-ground parts or water-filled tubers, adaptations in the root system to increase water absorption, and using trichomes (small hairs) on the leaves to absorb atmospheric water. However, drought remains a major cause of crop failure. In one embodiment, the genetically engineered plants are drought tolerant plants. As used herein, “drought tolerant” refers to plants having improved plant yield and fitness when exposed to abiotic plant stress, as compared to normal circumstances, and the ability of the plant to function and survive in such environments. Drought tolerant plants may also be referred to as “drought resistant” plants.

PDLP5-m5 plants were shown to have increased root length as well as an increase in total secondary root growth compared to WT plants (Example 1). Uga et al. have recently demonstrated that an increase in root branching increases drought tolerance in rice (2013 Nat Genet 45, 1097-1102). Without being bound by theory, it is believed that this change in root architecture might enable plant roots to reach water that is deeper in the ground and may thus be related to drought-resistant phenotype. In addition, the modified plasmodesmal connectivity enables plants to maintain constricted PD allowing water and/or nutrients to pass from cell to cell and maintain plant survival. Indeed, PDLP5-m5 plants also shown to have improved drought resistance (Example 1).

In some embodiments, increased tolerance to abiotic plant stress includes increased frost and/or cold tolerance. PDLP5-m5 plants were also exposed to cold conditions and demonstrated improved resistance relative to wild type plants and plants overexpressing PDLP5 (Example 1).

More generally, the invention provides a method of selecting for (or identifying) an alteration of an agronomic characteristic in a plant includes (a) obtaining a transgenic plant, wherein the transgenic plant includes in its genome a recombinant DNA construct including a polynucleotide operably linked to at least one regulatory sequence (for example, a promoter functional in a plant), wherein said polynucleotide encodes a modified PDLP protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines; (b) obtaining a progeny plant derived from said transgenic plant, wherein the progeny plant includes in its genome the recombinant DNA construct; and (c) selecting (or identifying) the progeny plant that exhibits an alteration in at least one agronomic characteristic when compared, optionally under water limiting conditions, to a control plant not including the recombinant DNA construct. The polynucleotide may encode a modified PDLP5 protein. The modified PDLP5 protein may confer increased stress tolerance.

In another embodiment, the invention provides a method of selecting for (or identifying) an alteration of at least one agronomic characteristic in a plant includes: (a) obtaining a transgenic plant, wherein the transgenic plant includes in its genome a recombinant DNA construct including a polynucleotide operably linked to at least one regulatory element, wherein said polynucleotide encodes a modified PDLP5 protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines, wherein the transgenic plant includes in its genome the recombinant DNA construct; (b) growing the transgenic plant of part (a) under conditions wherein the polynucleotide is expressed; and (c) selecting (or identifying) the transgenic plant of part (b) that exhibits an alteration of at least one agronomic characteristic when compared to a control plant not including the recombinant DNA construct. Optionally, said selecting (or identifying) step (c) includes determining whether the transgenic plant exhibits an alteration of at least one agronomic characteristic when compared, under water limiting conditions, to a control plant not including the recombinant DNA construct. The at least one agronomic trait may be yield, biomass, or both and the alteration may be an increase.

In some embodiments, the invention provides a method of selecting for (or identifying) an alteration of an agronomic characteristic in a plant, includes: (a) obtaining a transgenic plant, wherein the transgenic plant includes in its genome a recombinant DNA construct including a polynucleotide operably linked to at least one regulatory element, wherein said polynucleotide includes a nucleotide sequence, wherein the nucleotide sequence is: (i) hybridizable under stringent conditions with a DNA molecule including the full complement of SEQ ID NO:5; or (ii) derived from SEQ ID NO:5 by alteration of one or more nucleotides by at least one method selected from the group consisting of: deletion, substitution, addition and insertion; (b) obtaining a progeny plant derived from said transgenic plant, wherein the progeny plant includes in its genome the recombinant DNA construct; and (c) selecting (or identifying) the progeny plant that exhibits an alteration in at least one agronomic characteristic when compared, optionally under water limiting conditions, to a control plant not including the recombinant DNA construct. The polynucleotide may encode a modified PDLP5 protein. The modified PDLP5 protein may confer increased stress tolerance.

In some embodiments, a method of increasing drought tolerance in a plant includes: (a) introducing into a regenerable plant cell a recombinant DNA construct including a polynucleotide operably linked to at least one regulatory sequence (for example, a promoter functional in a plant), wherein the polynucleotide encodes a modified PDLP protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines; and (b) regenerating a transgenic plant from the regenerable plant cell after step (a), wherein the transgenic plant includes in its genome the recombinant DNA construct and exhibits increased drought tolerance when compared to a control plant not including the recombinant DNA construct. The method may further include (c) obtaining a progeny plant derived from the transgenic plant, wherein said progeny plant includes in its genome the recombinant DNA construct and exhibits increased drought tolerance when compared to a control plant not including the recombinant DNA construct.

In some embodiments, a method of increasing drought tolerance includes: (a) introducing into a regenerable plant cell a recombinant DNA construct including a polynucleotide operably linked to at least one regulatory element, wherein said polynucleotide includes a nucleotide sequence, wherein the nucleotide sequence is: (a) hybridizable under stringent conditions with a DNA molecule including the full complement of SEQ ID NOs:3 or 5; or (b) derived from SEQ ID NO:5 by alteration of one or more nucleotides by at least one method selected from the group consisting of: deletion, substitution, addition and insertion; and (b) regenerating a transgenic plant from the regenerable plant cell after step (a), wherein the transgenic plant includes in its genome the recombinant DNA construct and exhibits increased drought tolerance when compared to a control plant not including the recombinant DNA construct. The method may further include (c) obtaining a progeny plant derived from the transgenic plant, wherein said progeny plant includes in its genome the recombinant DNA construct and exhibits increased drought tolerance, when compared to a control plant not including the recombinant DNA construct.

In some embodiments, a method of selecting for (or identifying) increased drought tolerance in a plant, includes (a) obtaining a transgenic plant, wherein the transgenic plant includes in its genome a recombinant DNA construct including a polynucleotide operably linked to at least one regulatory sequence (for example, a promoter functional in a plant), wherein said polynucleotide encodes a modified PDLP5 protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines; (b) obtaining a progeny plant derived from said transgenic plant, wherein the progeny plant includes in its genome the recombinant DNA construct; and (c) selecting (or identifying) the progeny plant with increased drought tolerance compared to a control plant not including the recombinant DNA construct.

In another embodiment, a method of selecting for (or identifying) increased drought tolerance in a plant, includes: (a) obtaining a transgenic plant, wherein the transgenic plant includes in its genome a recombinant DNA construct including a polynucleotide operably linked to at least one regulatory element, wherein said polynucleotide encodes a modified PDLP protein having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines; (b) growing the transgenic plant of part (a) under conditions wherein the polynucleotide is expressed; and (c) selecting (or identifying) the transgenic plant of part (b) with increased drought tolerance compared to a control plant not including the recombinant DNA construct.

In some embodiments, a method of selecting for (or identifying) increased drought tolerance in a plant includes: (a) obtaining a transgenic plant, wherein the transgenic plant includes in its genome a recombinant DNA construct including a polynucleotide operably linked to at least one regulatory element, wherein said polynucleotide includes a nucleotide sequence, wherein the nucleotide sequence is: (i) hybridizable under stringent conditions with a DNA molecule including the full complement of SEQ ID NO:5; or (ii) derived from SEQ ID NO:5 by alteration of one or more nucleotides by at least one method selected from the group consisting of: deletion, substitution, addition and insertion; (b) obtaining a progeny plant derived from said transgenic plant, wherein the progeny plant includes in its genome the recombinant DNA construct; and (c) selecting (or identifying) the progeny plant with increased drought tolerance, when compared to a control plant not including the recombinant DNA construct.

### Pathogen Tolerant Plants

Plant pathogens can spread rapidly over great distances assisted by, for example, water, wind, insects, and/or humans. Across large regions and many crop species, it is estimated that diseases typically reduce plant yields by 10% every year in more developed nations or agricultural systems, but yield loss to diseases often exceeds 20% in less developed settings, an estimated 15% of global crop production. Plants pathogens infect plants by moving through PD.

Plants having increased stress tolerance may be tolerant to pathological plant stress. Biotic stresses include any infectious disease caused by a plant pathogen. As used herein, “pathogen tolerant” refers to a plant having improved plant yield and fitness when exposed to pathological plant stress, as compared to normal circumstances, and the ability of the plant to function and survive when exposed to pathological plant stress. Pathogen tolerant plants may also be referred to as “pathogen resistant” plants. The term “plant pathogen” includes any microorganism including, for example, fungi, oomycetes, bacteria, viruses, viroids, virus-like organisms, phytoplasmas, protozoa, nematodes and parasitic plants that can cause infectious disease in its host. Examples of bacterial plant pathogens include, without limitation, Pseudomonas syringae pv. tomato, Pseudomonas syringae pathovars, Ralstonia solanacearum, Agrobacterium tumefaciens, Xanthomonas oryzae pv. oryzae, Xanthomonas campestris pathovars, Xanthomonas axonopodis pathovars, Erwinia amylovora, Xylella fastidiosa, Dickeya (dadantii and solani), Pectobacterium carotovorum, Pectobacterium atrosepticum, Clavibacter michiganensis, Clavibacter sepedonicus, Pseudomonas savastanoi, and Candidatus Liberibacter asiaticus. Examples of viral plant pathogens include, without limitation, Tobacco mosaic virus, Cucumber mosaic virus, Brome mosaic virus, Tomato spotted wilt virus, Beet yellows virus, Citrus tristeza virus. Examples of fungal plant pathogens include, without limitation, ascomycetes (such as Fusarium spp., Thielaviopsis spp., Verticillium spp., Magnaporthe grisea, Sclerotinia sclerotiorum), and basidiomycetes (such as Ustilago spp., Rhizoctonia spp., Phakospora pachyrhizi, Puccinia spp., and Armillaria spp.).

Provided herein are methods for increasing pathogen resistance. In some embodiments, the methods for increasing pathogen resistance include providing a plant having increased stress tolerance, and growing the plant under exposure to the pathogen. In other embodiments, the methods for increasing pathogen resistance include providing a plant seed having increased stress tolerance, and growing the plant seed under exposure to the pathogen.

PD permeability is integrated into innate immune response and that this process is mediated by PDLP5 (Lee et al., 2011 Plant Cell 3353-3373). Specifically, PDLP5 plays a positive role in plant defense responses. Salicylic acid (SA) is a phenolic phytohormone and is found in plants with roles in plant growth and development, photosynthesis, transpiration, ion uptake and transport. SA plays a role in the resistance to pathogens by inducing the production of pathogenesis-related proteins and is involved in the systemic acquired resistance (SAR) in which a pathogenic attack on one part of the plant induces resistance in other parts. PDLP5 expression was induced by bacterial infection, suggesting that the regulation of PD constitutes a part of the innate immune response (Lee et al., 2011 Plant Cell 3353-3373). PDLP5 expression is induced by a salicylic acid (SA)-dependent signaling pathway activated by microbial infection, but PDLP5 also functions in a regulatory circuit via feedback amplification of SA, escalating immune responses while imposing a blockage of overall cytoplasmic coupling among cells (Lee et al., 2011 Plant Cell 3353-3373). The SA content in Arabidopsis pdlp5-1 plants having a severe knock-down in PDLP5 was similar to the wild-type control; however, Arabidopsis plants overexpressing PDLP5 accumulated 15-fold higher total SA compared with the wild-type control (Lee et al., 2011 Plant Cell 3353-3373) and exhibited innate immunity to Pseudomonads infection. Lee et al. (2011 Plant Cell 3353-3373) also provide evidence that a positive feedback regulatory loop exists between PDLP5 expression and accumulation of SA.

Without being bound by theory, it is believed that modified plasmodesmal connectivity enables plants to maintain constricted PD allowing water and/or nutrients to pass from cell to cell and maintain plant survival.

### Additional Methods for Producing Plants and Seeds

The invention further provides a method of producing a plant that exhibits at least one trait selected from the group consisting of: increased drought tolerance, increased yield, increased biomass, increased cold tolerance, early flowering and altered root architecture, wherein the method comprises growing a plant from a seed comprising a recombinant DNA construct, wherein the recombinant DNA construct comprises a polynucleotide operably linked to at least one regulatory element, wherein the polynucleotide encodes a modified PDLP5 protein having an amino acid sequence of at least 70%, 75%, 80%, 85%, 90%, 91%, 92%, 93%, 94%, 95%, 96%, 97%, 98%, 99% or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines, wherein the plant exhibits at least one trait selected from the group consisting of: increased drought tolerance, increased yield, increased biomass, increased cold tolerance, early flowering and altered root architecture, when compared to a control plant not comprising the recombinant DNA construct.

The polypeptide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

One may evaluate altered root architecture in a controlled environment (e.g., greenhouse) or in field testing. The evaluation may be under limiting or non-limiting water conditions. The evaluation may be under simulated or naturally-occurring low or high nitrogen conditions. The altered root architecture may be an increase in root mass. The increase in root mass may be at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45% or 50%, when compared to a control plant not comprising the recombinant DNA construct.

The invention also provides use of a recombinant DNA construct for producing a plant that exhibits at least one trait selected from the group consisting of: increased drought tolerance, increased yield, increased biomass, increased cold tolerance, early flowering and altered root architecture, when compared to a control plant not comprising said recombinant DNA construct, wherein the recombinant DNA construct comprises a polynucleotide operably linked to at least one regulatory element, wherein the polynucleotide encodes a modified PDLP5 protein having an amino acid sequence of at least 70%, 75%, 80%, 85%, 90%, 91%, 92%, 93%, 94%, 95%, 96%, 97%, 98%, 99% or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines. The polypeptide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

The invention also provides a method of producing a seed includes (a) crossing a first plant with a second plant, wherein at least one of the first plant and the second plant includes a recombinant DNA construct, wherein the recombinant DNA construct includes a polynucleotide operably linked to at least one regulatory element, wherein the polynucleotide encodes a modified PDLP5 protein having an amino acid sequence of at least 70%, 75%, 80%, 85%, 90%, 91%, 92%, 93%, 94%, 95%, 96%, 97%, 98%, 99% or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines; and (b) selecting a seed of the crossing of step (a), wherein the seed includes the recombinant DNA construct. A plant grown from the seed may exhibit at least one trait selected from the group consisting of: increased drought tolerance, increased yield, increased biomass, increased cold tolerance, early flowering and altered root architecture, when compared to a control plant not including the recombinant DNA construct. The modified PDLP5 protein may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

In some embodiments, the invention provides a method of producing seed (for example, seed that can be sold as a drought tolerant product offering) includes any of the preceding methods, and further including obtaining seeds from said progeny plant, wherein said seeds include in their genome said recombinant DNA construct.

In some embodiments, the invention provides a method of producing oil or a seed by-product, or both, from a seed includes extracting oil or a seed by-product, or both, from a seed that includes a recombinant DNA construct, wherein the recombinant DNA construct includes a polynucleotide operably linked to at least one regulatory element, wherein the polynucleotide encodes a modified PDLP having an amino acid sequence of at least 70%, 75%, 80%, 85%, 90%, 91%, 92%, 93%, 94%, 95%, 96%, 97%, 98%, 99% or 100% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, preferably 0 or 1 cysteine; more preferably no cysteines. The seed may be obtained from a plant that includes the recombinant DNA construct, wherein the plant exhibits at least one trait selected from the group consisting of: increased drought tolerance, increased yield, increased biomass, increased cold tolerance, early flowering and altered root architecture, when compared to a control plant not including the recombinant DNA construct. The modified PDLP5 protein may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass. The oil or the seed by-product, or both, may include the recombinant DNA construct.

Methods of isolating seed oils are well known in the art: (Young et al., Processing of Fats and Oils, In The Lipid Handbook, Gunstone et al., eds., Chapter 5 pp 253 257; Chapman & Hall: London (1994)). Seed by-products include but are not limited to the following: meal, lecithin, gums, free fatty acids, pigments, soap, stearine, tocopherols, sterols and volatiles.

In any of the preceding methods or any other embodiments of methods of the invention, in said introducing step said regenerable plant cell may include a callus cell, an embryogenic callus cell, a gametic cell, a meristematic cell, or a cell of an immature embryo. The regenerable plant cells may derive from an inbred maize plant.

In any of the preceding methods or any other embodiments of methods of the invention, said regenerating step may include (i) culturing said transformed plant cells in a media including an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the invention, the at least one agronomic characteristic may be selected from the group consisting of: abiotic stress tolerance, greenness, stay-green, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen stress tolerance, nitrogen uptake, root lodging, root mass, harvest index, stalk lodging, plant height, ear height, ear length, salt tolerance, cold tolerance, early flowering, early seedling vigor and seedling emergence under low temperature stress. The alteration of at least one agronomic characteristic may be an increase, e.g., in drought tolerance, yield, stay-green or biomass (or any combination thereof), or a decrease, e.g., in root lodging.

In any of the preceding methods or any other embodiments of methods of the invention, the plant may exhibit the alteration of at least one agronomic characteristic when compared, under water limiting conditions, to a control plant not including said recombinant DNA construct.

In any of the preceding methods or any other embodiments of methods of the invention, alternatives exist for introducing into a regenerable plant cell a recombinant DNA construct including a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the invention.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the invention containing a desired polypeptide is cultivated using methods well known to one skilled in the art.

Methods known to the art for making and using recombinant DNA constructs and transgenic plants, such as drought tolerant plants, are exemplified in Allen et al., US Pat Pubs. 20140245497, published Aug. 28, 2014, and 20120023622, published Jan. 26, 2012. Exemplary methods relating to PDLP5 expression are found in Lee et al., The Plant Cell, Vol. 23: 3353-3373, 2011.

In the preceding description, particular embodiments may be described in isolation for clarity. For example, several different plant types may be described in one section of the description, while several different proteins or biological sources of proteins may be described in another section of the description. It is expected that one of skill in the art will understand, that the description is explicitly intended to convey, that the various plant types described may be used in combination with the various proteins and/or biological sources of proteins, individually or collectively, in any reasonable conceivable combination to effect the biological production of the genetically engineered plant described herein. Unless it is otherwise expressly specified that the features of one particular embodiment are incompatible with the features of another embodiment, the invention is intended to encompass embodiments which include a combination of two or more compatible features described herein in connection, regardless of the textual position of the description of those embodiments within the document.

Moreover, it should be understood that preceding description is not intended to disclose every embodiment or every implementation of the present invention. The description more particularly exemplifies illustrative embodiments. For example, certain genes and plants are described herein. However, it should be understood that what is important is that the genetically engineered plant possess the designated improved yield and fitness; the actual biological source of those activities is not determinative or limiting and can be determined by the skilled artisan based on availability or convenience. In several places throughout the application, guidance is provided through lists of examples, which examples can be used in various combinations. In each instance, the recited list serves only as a representative group and should not be interpreted as an exclusive list.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Synthesis and Expression of PDLP5-m5

### Materials and Methods

A. thaliana transformed with p35S:PDLP5 (PDLP5-OX) which overexpresses PDLP5 under the 35S promoter, was prepared, and a TNA-insertion mutant, pldp5-1 (a severe knockdown strain) was isolated, as described in Lee et al., 2011 Plant Cell 23:3353-3373.

Using standard genetic engineering techniques, a PDLP5-m5 coding sequence containing 3 Cys→3 A1a mutations at positions 288, 289, and 298 of the C-terminal tail region of PDLP5 was cloned. Primers used to clone PDLP5-m5 by overlapping PCR are shown in Table 2. The 3′ primers contain missense mutations encoding the substituted Ala residues. A pSCB-PDLP5 cDNA clone was used as the PCR template. The resulting PCR products were cloned using pENTR/D-TOPO kit.

TABLE 2
Primers used to clone PDLP5-m5 by overlapping PCR

| Primer | | | SEQ |
|---|---|---|---|
| name | Description | Sequence (5′ → 3′) | ID NO: |
| PDLP5- | 5′ Forward | caccgaatcATGATCAAGACAAAGACGACGTC | 9 |
| GWF 1 | | | |
| PDLP5- | 3′ Reverse: nested | TCATCTTGTAATTTTCTAGCAGCCTTTCCAACAAAAGCGAG | 10 |
| GWR5 | 3′end of PDLP5 CT | | |
| | 3C −> 3A) | | |
| PDLP5- | 3′ Reverse: 3′end of | TCATTTAGCCCATTTCTCATCTTGTAATTTTC | 11 |
| GWR6* | PDLP5 CT 3C −> 3A) | | |

The sequence of the PCR product was confirmed followed by transformation of Top10 E. coli competent cells. After sequence confirmation, the PDLP5-m5 sequence was transferred from the entry clone pEN-PDLP5-m5 into a binary vector (pGWB) including the constitutive 35S promoter by a Gateway reaction; more particularly, the sequence was cloned into an expression vector under the control of a constitutive 35S promoter to create a pGWB-35S:PDLP5-m5 clone. Agrobacteria were transformed with the pGWB-35S:PDLP5-m5 clone. More particularly, pGWB was introduced into Agrobacteria strain GV3101, and the transformed Agrobacteria were used to transform 4-week-old Arabidopsis thaliana Col-0 plants using floral dipping to produce transgenic plants expressing PDLP5-m5. Transgenic Arabidopsis plants expressing the PDLP5-m5 mutant (pro35S:PDLP5-m5) were made in Col-0 WT and pldp5-1 (severe knockdown) backgrounds.

Transgenic T1 plants were selected on basta+plates and homozygous T2 lines were identified by segregation test on T3 plants. For the drought test, homozygous lines were grown in soil for the 2 weeks with watering followed by 2 weeks of water withdrawal. Tolerance to drought stress of the PDLP5-m5 was assessed by the plant growth or death upon resuming watering.

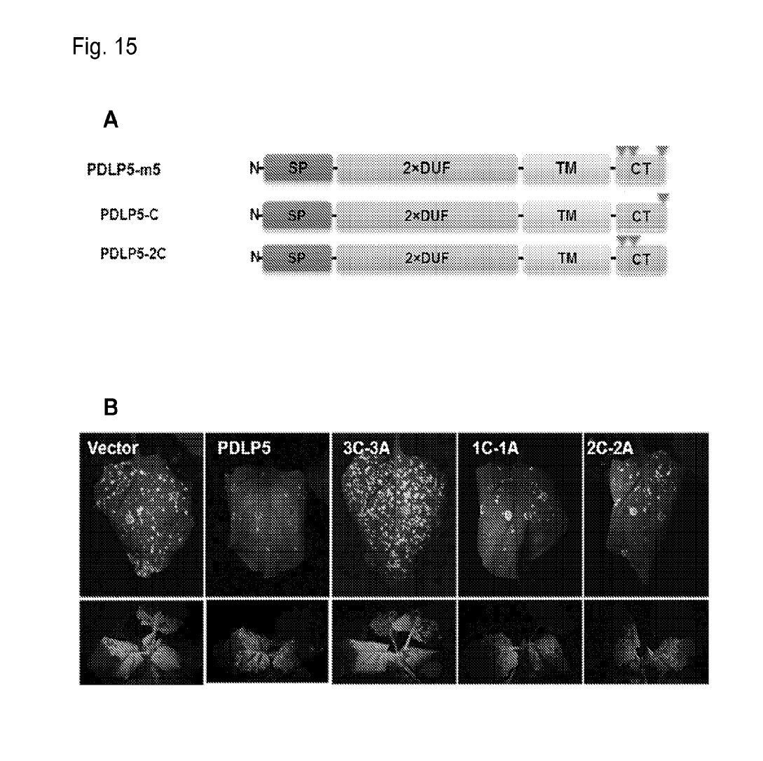

We also created mutants altering just two cysteines (“CC”, residues 288 and 289 of the C-terminal tail region) or a single cysteine (“C”, residue 298 of the C-terminal tail region) residues to A1a. The phenotypes of these partial mutants, as well as the PDLP5-OX, pldp5-1 and PDLP5-m5, were tested in transient expression in Nicotiana benthamiana leaves via Agrobacteria-infiltration, targeting the extent of viral movement for comparison. PDLP5-m5 overexpression results in more extensive viral movement, whereas PDLP5 WT protein overexpression results in a delay in viral movement. PDLP5-CC or PDLP5-C mutants did not show PDLP5-m5 effect (FIG. 15).

### Results and Discussion

PDLP5-m5 expression in a wild type background exhibits normal basal levels of expression and exhibits open PD, similar to wild type plants. In response to plant stress, PDLP5-m5 expression is induced as is seen in wild-type PDLP5; however, the PDLP5-m5 function may be altered relative to wild type PDLP5.

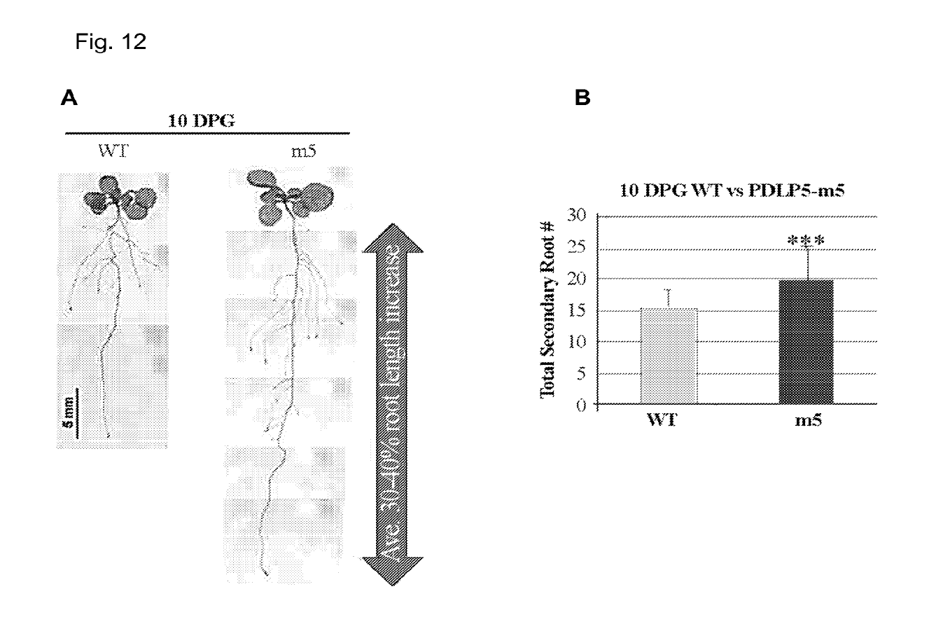

PDLP5-m5 plants exhibit a 30-40% increase in root length (FIG. 12A) as well as an increase in total secondary root growth (FIG. 12B), compared to wild-type plants.

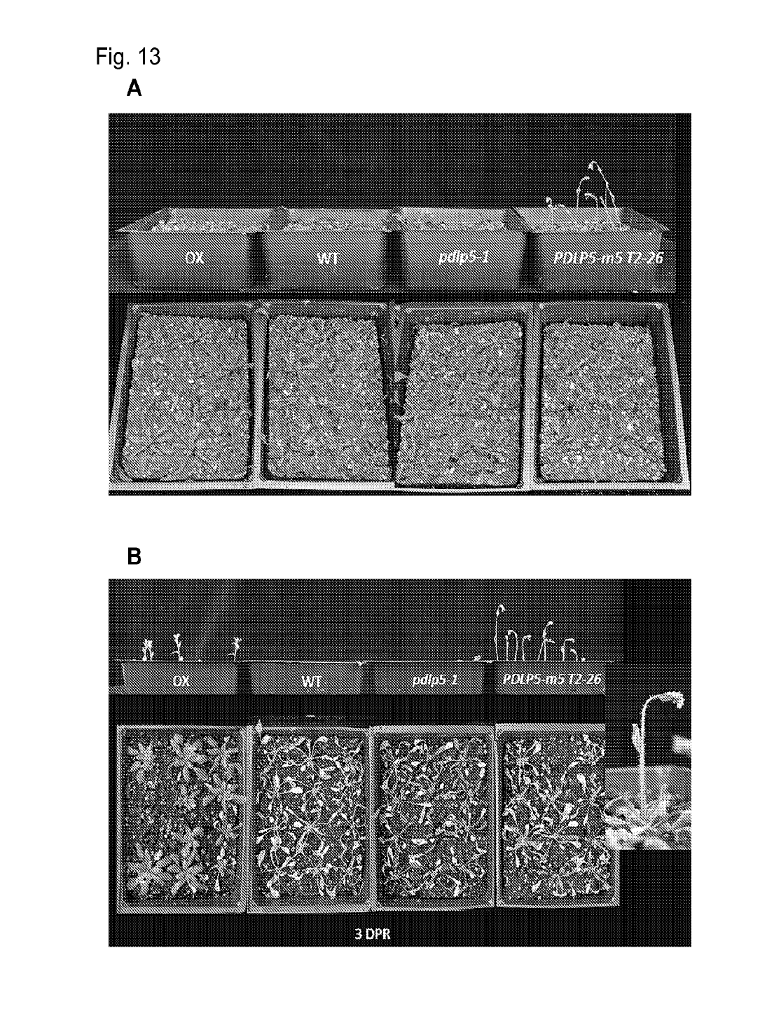

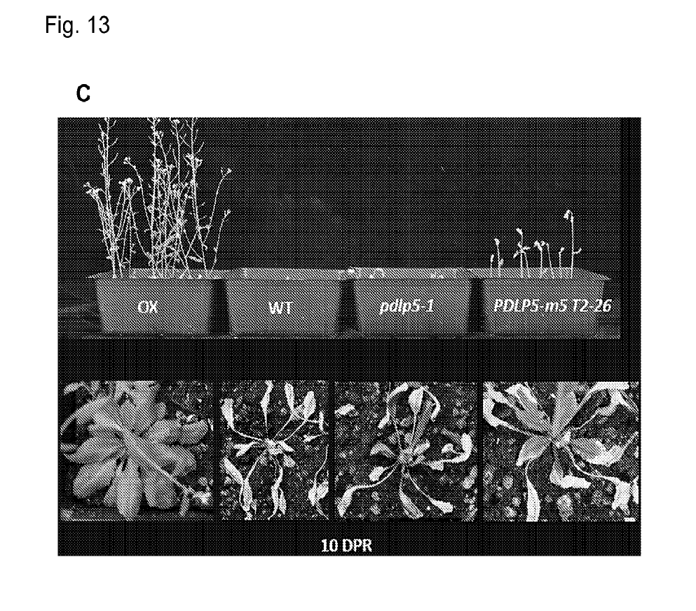

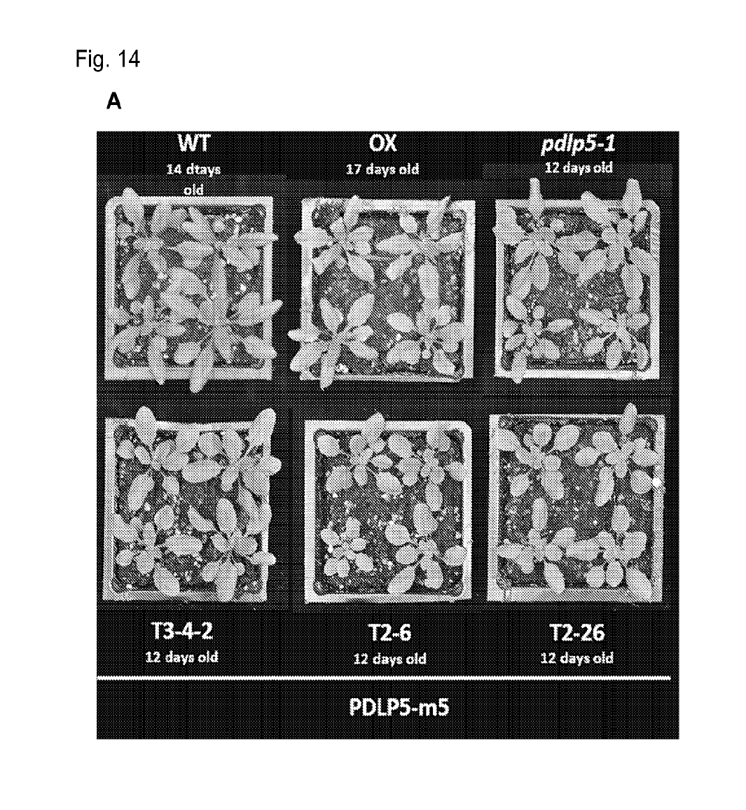

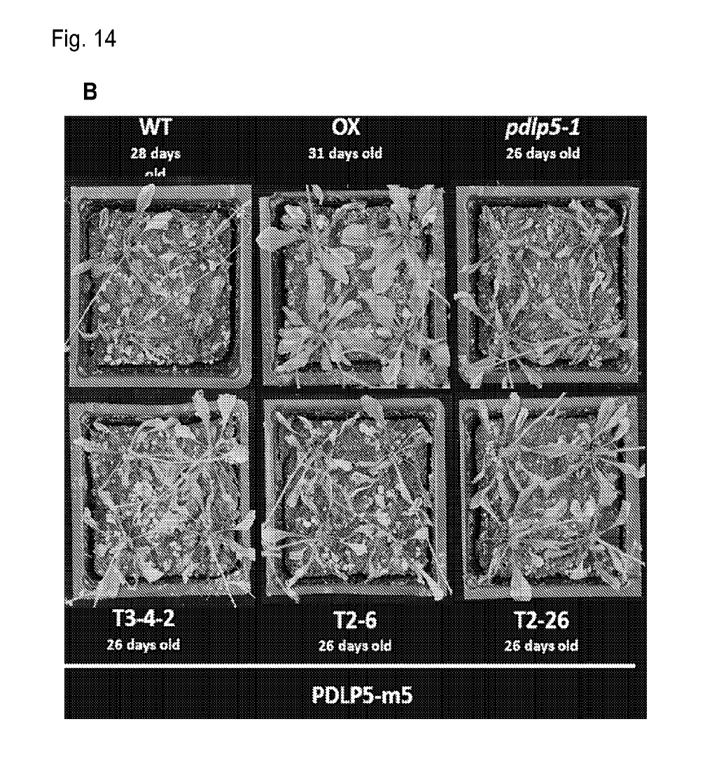

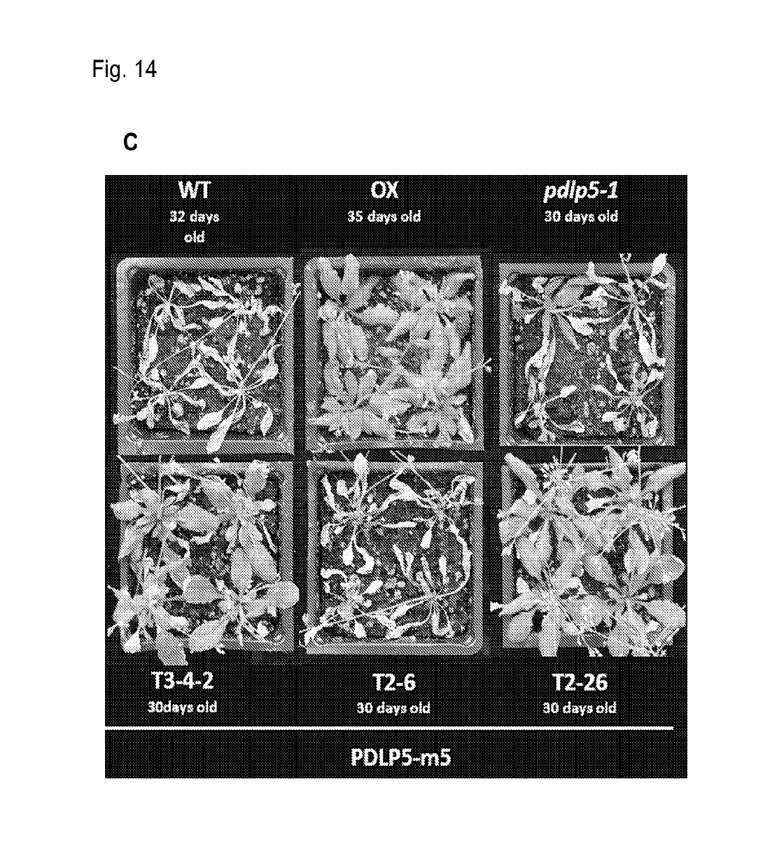

PDLP5-m5 plants were evaluated by exposing them to drought conditions. PDLP-m5 plants were grown under normal conditions with water, water was withdrawn for 2 weeks, and rewatering was commenced. Plants having a severe knock down of PDLP5 (pdlp5-1) and PDLP5-m5 plants bolt faster than plants overexpressing PDLP5 and wild type plants (FIG. 13A). PDLP5-m5 plants were resistant to water withdrawal (FIG. 13B) and continued to grow upon rewatering (FIG. 13C). Plants overexpressing PDLP5 recovered after rewatering (FIG. 13C). Wild type plants and plants having a severe knock down of PDLP5 (pdlp5-1) died as a result of the water withdrawal and were not able to recover following rewatering (FIG. 13B and FIG. 13C). The early bolting of the PDLP5-m5 plants appeared to affect plant revival. Therefore, the experiment was repeated using same day bolting samples. Briefly, plants having the same rosette size were used, regardless of the number of days required to achieve that rosette size (FIG. 14A). Again, PDLP5-m5 plants were resistant to water withdrawal (FIG. 14B) and continued to grow upon rewatering (FIG. 14C).

PDLP5-m5 plants were also exposed to cold conditions and demonstrated improved resistance relative to wild type plants and plants overexpressing PDLP5 (FIG. 11).

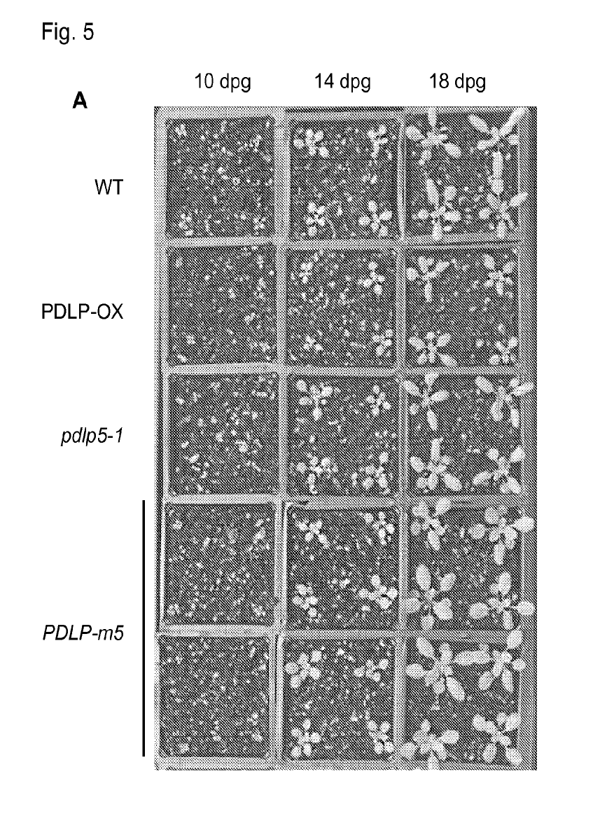

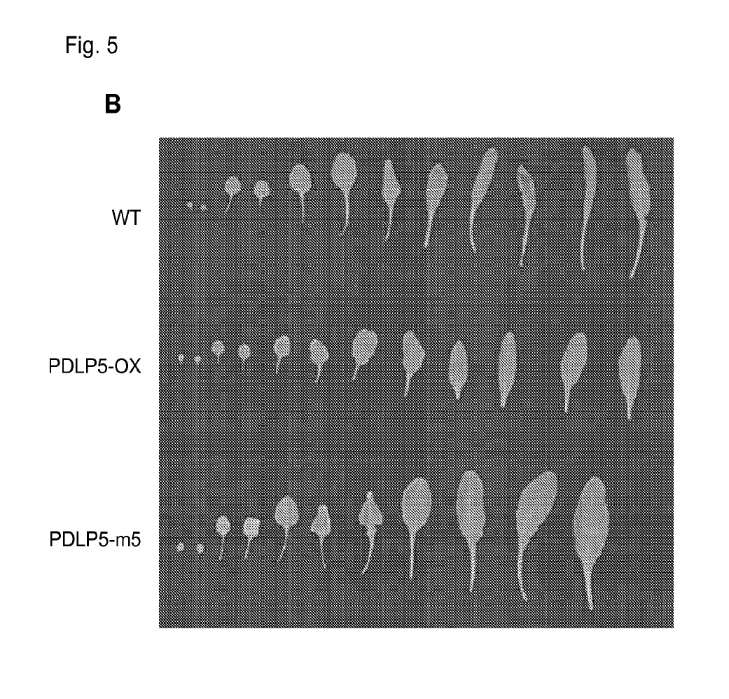

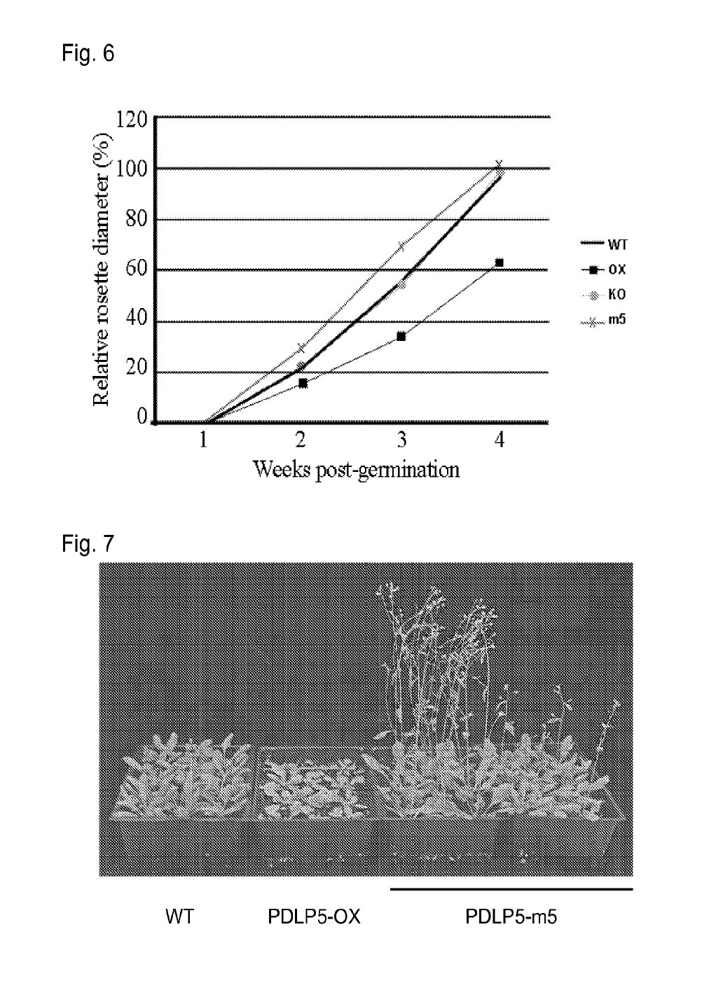

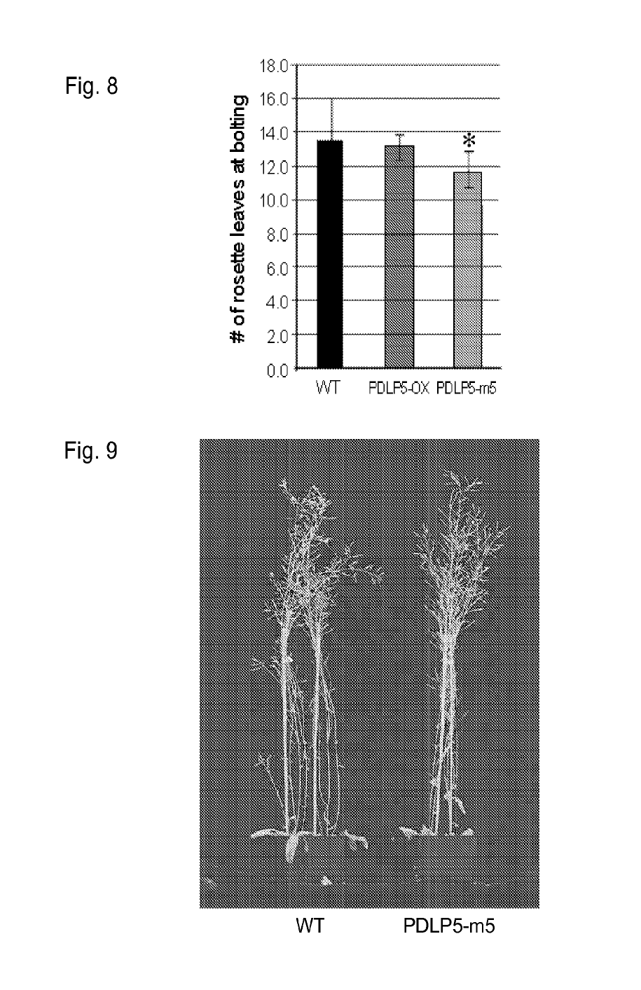

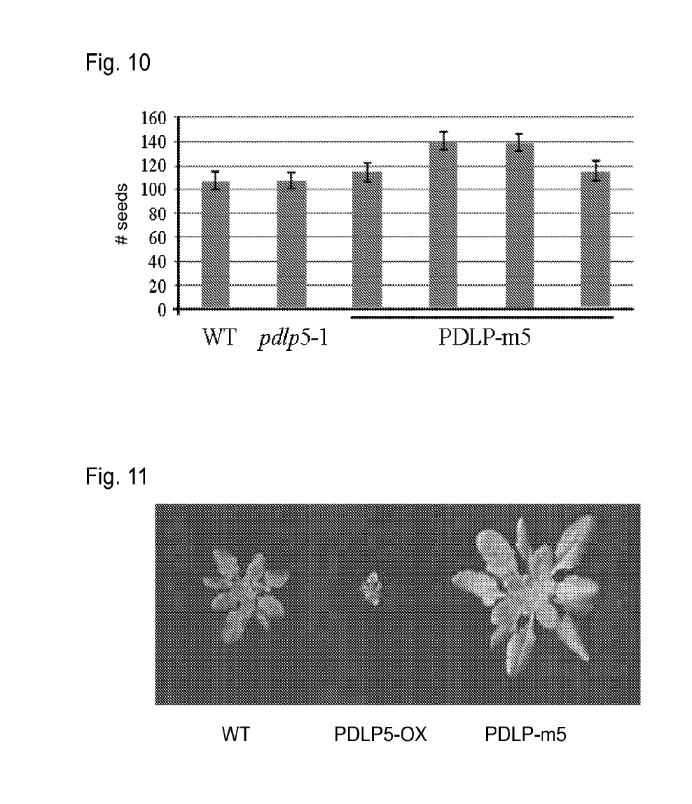

Thus, introducing PDLP5-m5 into Arabidopsis enhanced the vigor of plant growth. More specifically, introducing PDLP5-m5 into Arabidopsis confers increased stress tolerance (drought resistance and cold resistance; FIGS. 11, 13, and 14) and improved agronomic characteristics (increased vegetative growth, extensive root architecture, early flowering, and increased yield; FIGS. 5-10 and 12).

Induced PDLP5-m5 expression does not appear to close PD as is seen in wild type plants and in genetically engineered plants overexpressing PDLP5. Without intending to be limited by theory, it appears that plasmodesmata connectivity may be modified such that the flow of compounds back and forth between cells is limited, but still available.

### Example 2

### Auxin Induces Expression of Plasmodesmata Regulator PDLP5 in Cells Overlying New Lateral Roots to Control Organ Emergence

New lateral roots originate from stem cells deep within the primary root. Lateral root initiation, patterning and emergence require coordination between the new primordium and overlying cells through which it must emerge. To facilitate organ emergence the hormone auxin is released by lateral root primordia (LRP) and taken up by overlying cells, activating expression of wall remodeling enzymes that trigger cell separation. Here we report that auxin also controls the induction of PDLP5, a gene known to modulate plasmodesmata permeability, within cells overlying new LRP. LRP emergence is accelerated in a loss-of-function mutation of this gene, pdlp5-1, resulting in more extensive root branching, whereas ectopic overexpression of PDLP5 results in a delay in LRP emergence as well as a severe reduction in LR numbers. We propose that auxin-induced PDLP5 expression promotes symplastic isolation of the cells overlying new LRP, restricting intercellular auxin diffusion and controlling the rate of cell separation during organ emergence.

### Introduction

Lateral root (LR) emergence is a well-coordinated cell patterning process in plants, driven by auxin (Swamp et al. 2008 Nat Cell Biol 10(8):946-954; Peret et al. 2009 Trends Plant Sci 14(7):399-408). Following the initiation of LR primordia (LRP), new organs undergo multiple cell divisions and begin outward growth and development (Malamy and Benfey 1997 Development 124(1):33-44; Lucas et al. 2013 Proc Nall Acad Sci USA. 110(13):5229-34). During this later stage, growing LRP push through the cells in the outer layers and finally emerge from the main root (Peret et al. 2009 Trends Plant Sci 14(7):399-408). Many components of the LR emergence pathway have been identified through analyses of genetic mutants that lead to an aberrant number of LRs. These include the auxin influx carrier mutants auxin1 (aux1) and like aux1-3 (lax3), and transcriptional regulator mutants indole acetic acid 3 (iaa3)/short hypocotyl 2 (shy2), iaa14/solitary root (slr), and auxin response factors 7 (arf7) & 19 (arf19) (Peret et al. 2012 Plant Cell 24(7):2874-2885; Tian, 2002 Plant Cell 14(2):301-319; Fukaki et al. 2002 Plant J29(2):153-168; Okushima et al. 2005 Plant Cell 17(2):444-463). According to the current model, the tip of newly developing LRP release auxin to the overlying cells that are in direct contact with the LPR. This occurs in a highly localized manner to activate cell wall-remodeling (CWR) within the target overlying cells so that these cells can separate, allowing LRP to push through (Swamp et al. 2008 Nat Cell Biol 10(8):946-954; Gonzalez-Carranza et al. 2007 J Exp Bot 58(13):3719-3730). Key players in this process include LAX3 and ARF7, which act upstream of several CWR enzymes and newly identified signaling components, INFLORESCENCE DEFICIENT IN ABSCISION (IDA) and a leucine-rich repeat receptor-like kinase HAESA (HAE) and HAESA-LIKE2 (HSL2) (Kumpf et al. 2013 Proc Natl Acad Sci USA. 110(13):5235-40).

For the auxin-driven emergence process to occur across cellular boundaries in a highly localized, spatiotemporal manner, coordination between intra- and inter-cellular auxin signaling is critical. To date, attention has focused on studying the role of polar auxin transport during lateral root emergence (Swamp et al. 2008 Nat Cell Biol 10(8):946-954; Marhavy et al. 2013 EMBO J 32(1):149-158). Nevertheless, plant cells are inter-connected by pore like structures termed plasmodesmata (PD) that may also serve as a conduit for movement of signals like hormones. Recently, we reported the identification and characterization of the PD-located protein PDLP5, which closes PD as an innate immune response (Lee et al. 2011 Plant Cell 23(9):3353-3373). PDLP5 expression is induced by a salicylic acid (SA)-dependent signaling pathway activated by microbial infection, but PDLP5 also functions in a regulatory circuit via feedback amplification of SA, escalating immune responses while imposing a blockage of overall cytoplasmic coupling among cells. Under normal growth conditions, restriction of PD is compromised in pdlp5-1, causing an anomalously extensive basal cell-to-cell permeability. Surprisingly, this inability to control PD also led to an increased susceptibility. In addition, a gain-of-function mutant generated by ectopic over production of PDLP5 under the control of the 35S promoter, not only strictly closed PD but also boosted innate immunity, underscoring the critical role of cell-to-cell connectivity in mounting whole plant immune signaling.

In this study, we report that spatiotemporal expression of PDLP5 within cells overlying LRP is under the control of auxin, and its level of expression is negatively correlated with the rate of LR emergence. By employing auxin markers in the mutant pdlp5-1 and a PDLP5 over-expressor lines, we demonstrate that PDLP5 is specifically required for LR emergence stages but not for LR initiation. We conclude by proposing a model that spatiotemporal regulation of PDLP5 by auxin is necessary to ensure that optimal levels of auxin accumulate within LRP and overlying cells, thereby influencing organ emergence and subsequent growth.

### Results

### PDLP5 is Required for Normal Progression of Lateral Root Emergence and Branching

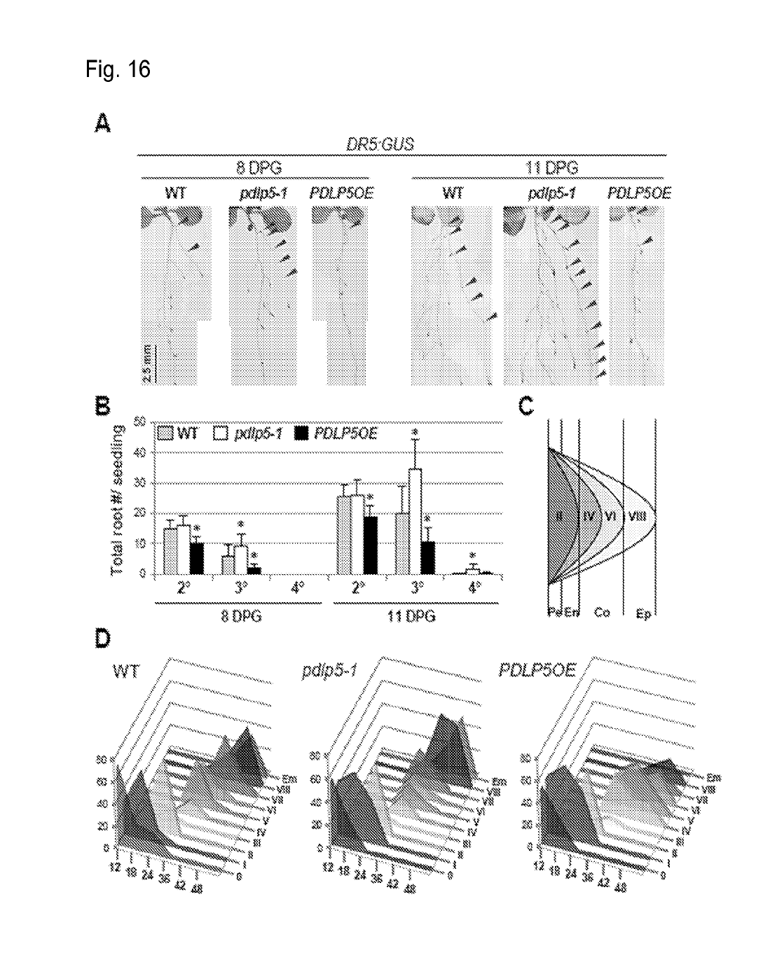

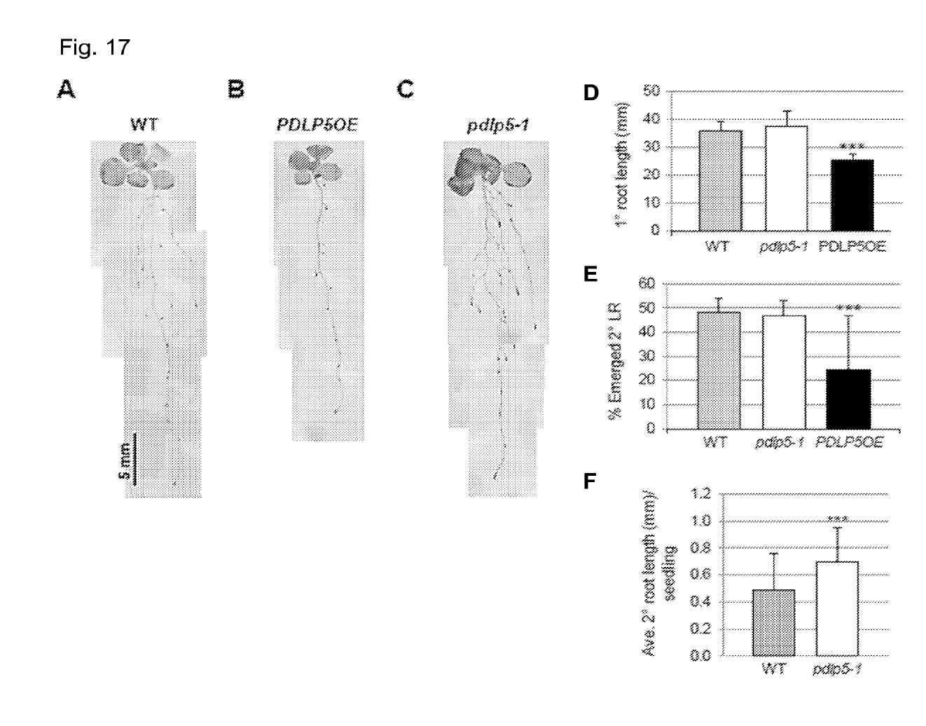

To investigate the importance of PD function during lateral root emergence, PDLP5 loss-of-function and over-expressing lines were characterized. Constitutive overexpression of PDLP5 under the 35S promoter (hereafter called PDLP5OE) exhibited reduced primary root growth and LR formation compared to WT (FIG. 16 A and FIG. 17A). Compared to wild type (WT) seedlings, ten-day-old PDLP5OE showed a reduction in the primary root length by approximately 30% (FIG. 17B) and produced significantly fewer secondary roots (only 66% and 77% of WT number at both 8 and 11 dpg, respectively) (FIG. 16A and B). Furthermore, this reduction in secondary root development was reiterated in later stages of root branching, with PDLP5OE showing reduced tertiary root occurrence (26% and 50% of WT at 8 and 11 day post germination [dpg], respectively). Notably, among the total secondary roots examined, the emerged LRP in PDLP5OE corresponded to only 50% of that found in WT (FIG. 17C). Conversely, pdlp5-1 exhibited enhanced lateral root length and branching pattern (FIG. 16A and FIG. 17A). Lateral roots were 1.4-fold longer on average in pdlp5-1 than WT (FIG. 17F) at 7 dpg and later. The total numbers of tertiary and quaternary roots were also higher in the pdlp5-1 background compared to WT, having about 33% more tertiary roots at 8 and 11 dpg, and almost twice as many quaternary roots by 11 dpg (FIG. 16B). Given the overall increase in average secondary root length in pdlp5-1, we conclude that the higher occurrence of tertiary and quaternary root formation can be attributed to an accelerated secondary root growth.

To determine whether PDLP5 plays a role in the initiation and/or emergence of LRP, we employed a bioassay that was previously developed to monitor the dynamics of LRP progression following an inductive gravitropic stimulus (Peret et al. 2012 Nat Cell Biol 14(10):991-998). In this assay, seedlings grown vertically on plates are subjected to gravistimulation by turning the plates 90° at 3 dpg, which triggers LRP formation in a highly synchronized temporal manner. Using this experimental setup, we compared the rates of LRP development in WT, PDLP5OE, and pdlp5-1. LRP undergoe eight stages of development, from its initiation at the xylem pole pericycle to emergence through overlying cell layers of endodermis, cortex, and epidermis (FIG. 16C) (Peret et al. 2012 Nat Cell Biol 14(10):991-998). Early LRP initiation and organization (stages 0-IV) during 12 to 36 hours post-gravitropic induction (hpg) in PDLP5OE or pdlp5-1 was similar to WT (FIG. 16D). However, during the later time period of 36-48 hpg corresponding to emergence stages VI-VIII, LRP in pdlp5-1 began to progress faster than those in WT. By 42 hpg, pdlp5-1 had 32% more LRP in stage VIII, and by 48 hpg, 17% more LRP had emerged in pdlp5-1 compared to WT. In contrast, progression of LRP in PDLP5OE was disrupted; by 48 hpg, PDLP5OE LRP were abnormally spread out across stages IV-VIII, and none had yet emerged unlike WT where most were at stage VIII or already emerged (FIG. 16D). Hence, PDLP5 appears to regulate LRP emergence but not organ initiation.

### PDLP5 Expression is Induced in Cells Overlying Lateral Root Primordia

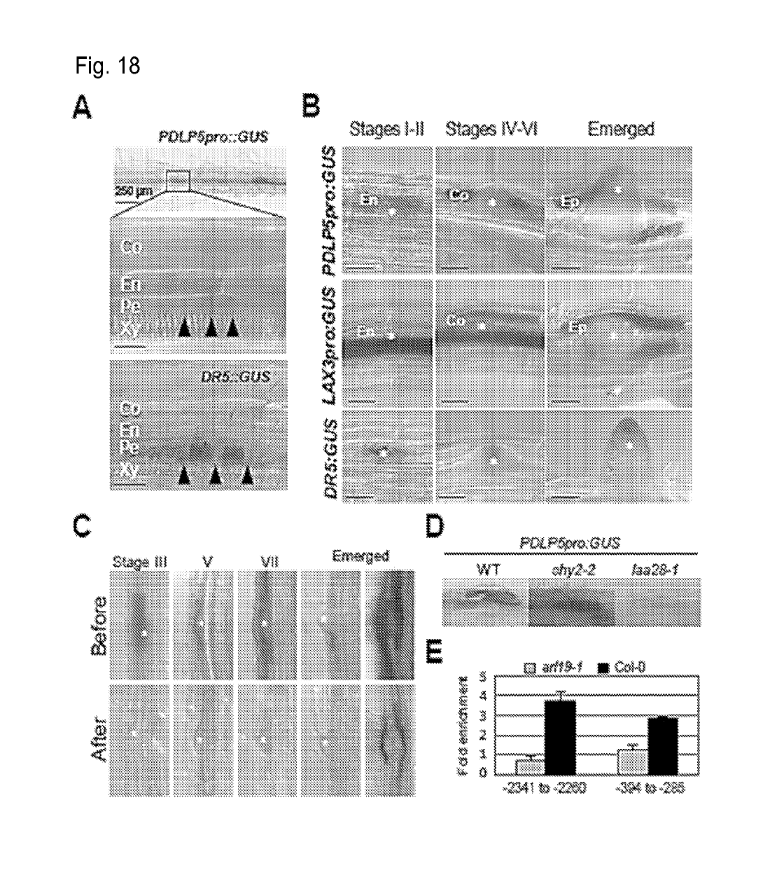

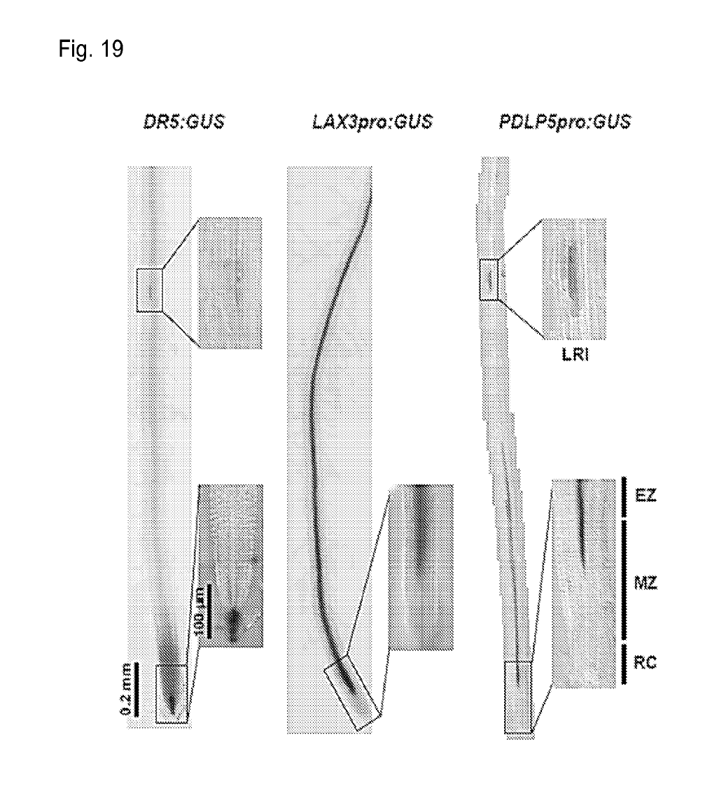

Given the LRP phenotypes exhibited in PDLP5 over expression and mutant lines, and the known role of PDLP5 in facilitating PD closure (Lee et al. 2011 Plant Cell 23(9):3353-3373), it is tempting to speculate that the rate of LRP emergence through overlying cell layers is sensitive to the extent of direct cell-to-cell coupling. If this were the case, endogenous PDLP5 expression may exhibit spatial regulation in cells overlying LRP, similar to the CWR enzymes xyloglucan:xyloglucosyl transferase (XTR6), subtilisin-like protease (AIR3), and polygalacturonase (PG) (Swamp et al. 2008 Nat Cell Biol 10(8):946-954; Gonzalez-Carranza et al. 2007 JExp Bot 58(13):3719-3730; Neuteboom et al. 1999 Plant Mol Biol 39(2):273-287; Vissenberg et al. 2005 J Exp Bot 56(412):673-683). To test this hypothesis, we examined PDLP5 expression in root tissues histochemically using PDLP5pro:GUS seedlings. Strong GUS staining was detected in the provasculature of the primary and lateral root tips but excluded from the meristematic zone (FIG. 19). More conspicuously, localized points of staining occurred along the length of primary and lateral roots, reminiscent of the positions of developing LRP. Microscopic analysis of those stained patches of cells at a higher magnification confirmed that the PDLP5po:GUS expression occurred in cells overlying LRP (FIG. 19). Specifically, PDLP5pro:GUS expression was detected in endodermal (En) cells overlying dividing xylem pole pericycle cells at the earliest stages of LRP development, followed by cortical (Co) and epidermal (Ep) cells overlying emerging LRP in a progressive manner (FIGS. 18A and B). PDLP5 expression pattern in Co and Ep cells resembled the auxin influx carrier LAX3 that controls LRP emergence (FIG. 18B) (Swamp et al. 2008 Nat Cell Biol 10(8):946-954). However, unlike the auxin response reporter DR5:GUS, PDLP5pro:GUS expression was excluded from the LR meristem similar to LAX3 (FIG. 18B and FIG. 19). Collectively, PDLP5 expression in roots occurs in a controlled manner within the cells above emerging LRP, throughout every developmental stage, consistent with its proposed role during organ emergence.

### Auxin, ARF19, IAA28 and SHY2 Control PDLP5 Expression in Cells Overlying New LRP

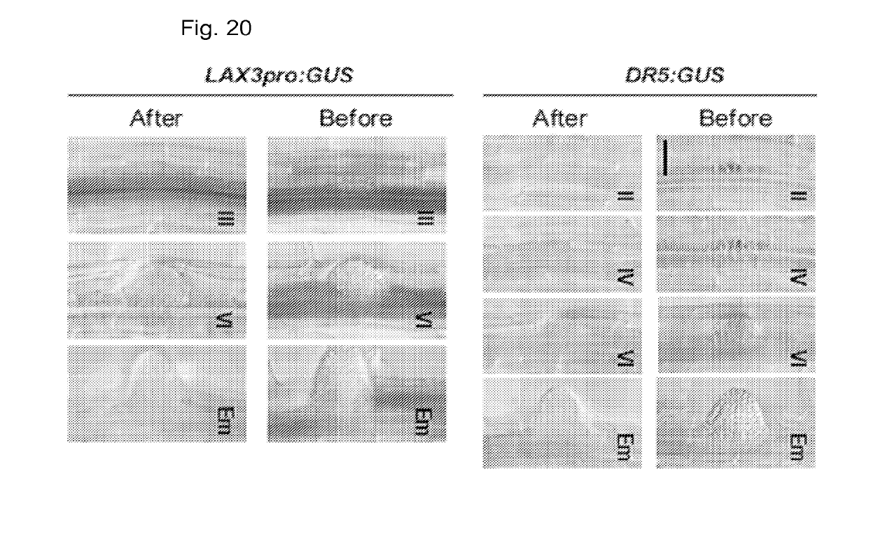

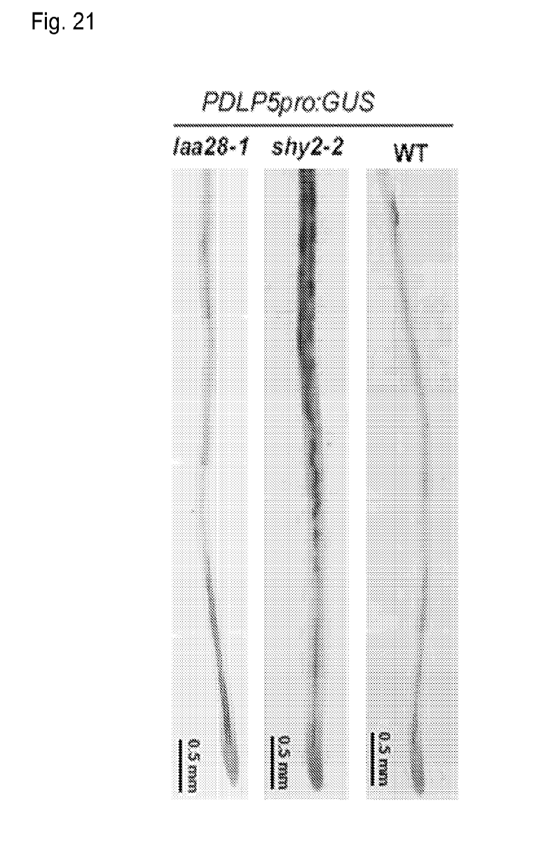

Regulation of the auxin-induced LAX3 gene with a similar expression pattern to PDLP5 in overlying cells of LPR has been shown to rely on auxin flow from the shoots (Swamp et al. 2008 Nat Cell Biol 10(8):946-954). Shoot-derived auxin is targeted to newly initiated LRP, providing a localized source of auxin for overlying cells. To test whether PDLP5 expression was also dependent on shoot-derived auxin, we grew PDLP5pro:GUS seedlings vertically on plates for five days, then excised the shoots and allowed roots to grow two more days before histochemical staining This treatment revealed a clear reduction in the activity of auxin-inducible PDLP5pro:GUS (FIG. 18C), and DR5:GUS and LAX3pro:GUS (FIG. 20) reporters.

To pinpoint which components of the auxin signaling pathway controls PDLP5 expression in roots, we expressed the PDLP5pro:GUS reporter in the auxin response mutants, iaa28-1 and shy2-2. LRP development is severely suppressed in iaa28-1 because it disrupts auxin-dependent founder cell specification (De Rybel et al. 2010 Curr Biol 20(19):1697-1706). In contrast, shy2-2 blocks auxin responses in the En cell layer, resulting in a high auxin in pericycle cells and increased LR formation but also causing an inhibition of LRP emergence (Goh et al., 2012 Philos T Roy Soc B 367(1595):1461-1468). GUS staining revealed PDLP5pro:GUS expression was decreased in cells above the few early-stage LRP that formed in iaa28-1 mutant background (FIG. 18D, 21). In contrast, PDLP5pro:GUS expression was strongly concentrated in En cells above the many aborted LRP in shy2-2.

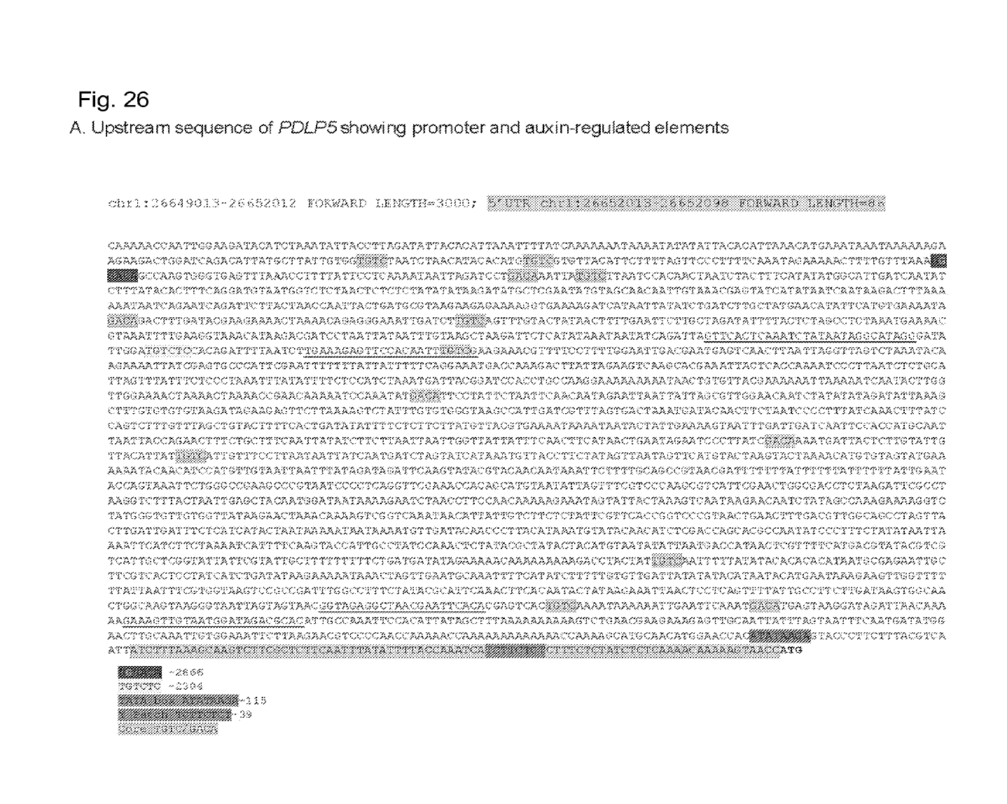

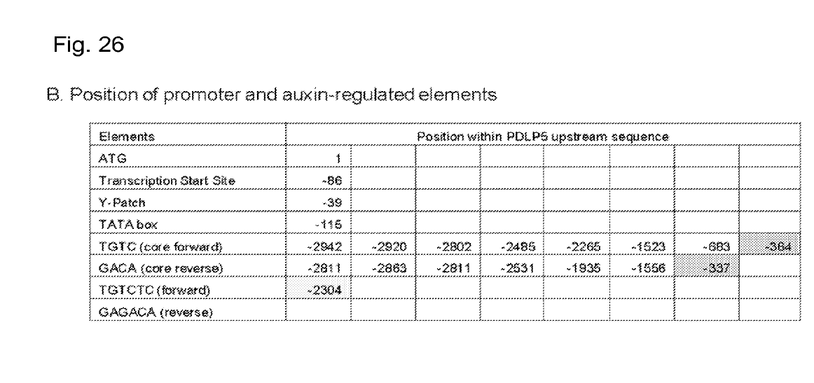

IAA28 and SHY2 encode repressors of transcription factors such as Auxin Reponse Factor 19 (ARF19) during LRP formation (Rogg et al. 2001 Plant Cell 13(3):465-480; Tian and Reed 1999 Development 126(4):711-721). Chromatin immunoprecipitation assays revealed that ARF19 binds to PDLP5 promoter segments containing either a canonical or core auxin responsive elements in WT but not arf19 mutant background (FIG. 18E and FIG. 26). Collectively, our data indicate that expression and correct patterning of PDLP5 in cells overlying new root organs requires auxin signaling pathway components ARF19, IAA28 and SHY2, which are known to control early LRP development and emergence (De Rybel et al. 2010 Curr Biol 20(19):1697-1706; Goh et al., 2012 Philos T Roy Soc B 367(1595):1461-1468; Rogg et al. 2001 Plant Cell 13(3):465-480; Tian and Reed 1999 Development 126(4):711-721).

### PDLP5 Modulates the Auxin-Dependent Induction of LAX3 in Cells Overlying New LRP

What is the role of PDLP5 in cells overlying new LRP? The current model explaining how overlying cells separate as newly formed LRP push through the internal cell layers predicts that auxin accumulating at the tip of LRP is transported into the extracellular matrix, from which directly overlying cells import auxin. This local accumulation of auxin then triggers a secondary auxin regulatory network within the overlying cells, inducing a subset of genes that control cell wall remodeling and separation (Swamp et al. 2008 Nat Cell Biol 10(8):946-954). Given the function of PDLP5 in restricting PD permeability (Lee et al. 2011 Plant Cell 23(9):3353-3373), the precise spatiotemporal control of its expression by auxin (FIG. 18A and B), and its impact on kinetics of LRP emergence (FIG. 16C), we hypothesized that PDLP5 functions as a component of this secondary auxin regulatory network.

To test this hypothesis, we monitored induction of a key component of the secondary auxin regulatory network, LAX3, by using LAX3pro:LAX3-YFP (Swamp et al. 2008 Nat Cell Biol 10(8):946-954) as a marker for auxin accumulation in PDLP5OE and pdlp5-1 backgrounds.

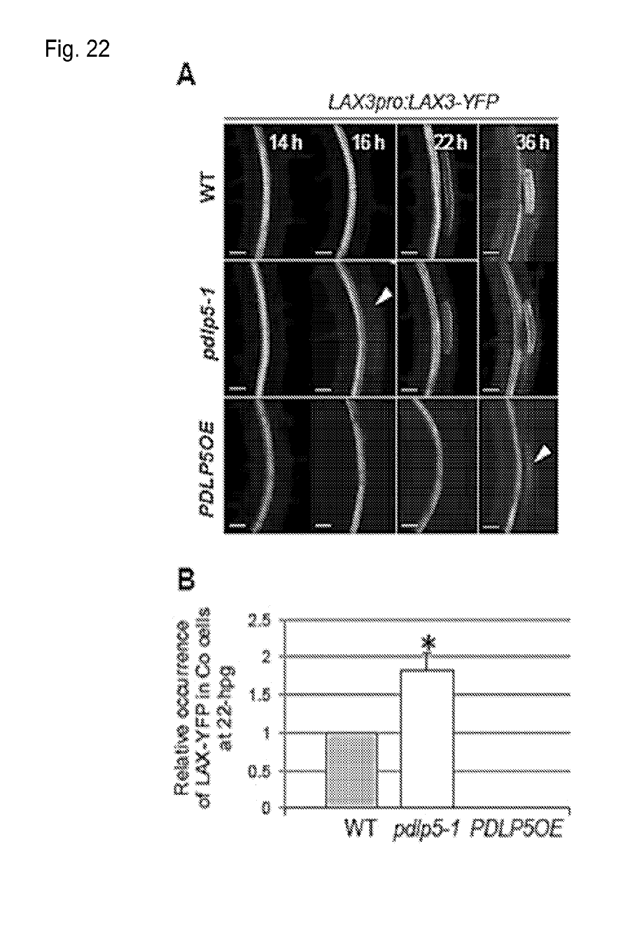

We monitored LAX3-YFP signal accumulation in overlying Co using the gravitropic assay. At 14 or 16 hpg, LRP have not yet reached Co cells in WT, and hence no LAX3-YFP fluorescent signals were detectable (FIG. 22A). At 22 hpg, 18% of WT seedlings began to accumulate LAX3-YFP signals in overlying Co cells and 100% by 36 hpg (FIG. 22A and Table 3). Detection of LAX3-YFP signals was much delayed in PDLP5OE Co cells, with no fluorescent signals evident at 22hrs (FIG. 22A and B). At 24 hpg, 33% seedlings were fluorescent in overlying Co cells, whereas 55% of WT seedlings showed LAX3-YFP signals by this time point (Table 4). By 36 hpg, all seedlings from WT or PDLP5OE background expressed the marker in overlying Co cells (Table 4). By contrast, Co cells in pdlp5-1 occasionally found to produce LAX3-YFP signals from as early as 16 hpg (FIG. 22A) and by 22 hpg, almost two-fold more Co cells expressed the marker compared to those in WT at this stage of LR development (FIG. 22B, Table 3). These results, consistent with the perturbation in LRP emergence kinetics observed in pdlp5-1 and PDLP5OE lines (FIG. 16D), provide strong evidence that pdlp5-1 achieves a faster buildup of auxin within overlying Co cells, whereas ectopic expression of PDLP5 significantly delays this process.

TABLE 3
Quantification of seedlings expressing LAX3pro:LAX3-YFP in overlaying
Co cells at 22 hours post-gravitropic stimulation.

| Repeats | WT | pdlp5-1 | PDLP5 | pdlp5-1:WT | PDLP5:WT |
|---|---|---|---|---|---|
| Set 1 | 6/23 (26%) | 9/20 (45%) | 0/21 (0%) | 1.73 | 0 |
| Set 2 | 5/43 (12%) | 10/40 (25%) | 0/23 (0%) | 2.08 | 0 |
| Set 3 | 7/34 (21%) | 13/37 (35%) | 0/24 (0%) | 1.67 | 0 |
| Total # of | 100 | 97 | 68 | | |
| seedlings | | | | | |
| Average | 18% | 33% | 0% | 1.83 | 0 |

TABLE 4
LAX3pro:LAX3-YFP cortical signal in PDLP5 at
24 and 26 hours post-gravitropic response.

| | WT | PDLP5 | PDLP5:WT | PDLP5:WT |
|---|---|---|---|---|
| Repeats | (24 hpg) | (24 hpg) | (24 hpg) | (36 hpg) |
| Set 1 | 11/31 (36%) | 4/27 (15%) | 0.42 | 1 |
| Set 2 | 22/30 (73%) | 15/30 (50%) | 0.68 | 1 |
| Total # of | 61 | 57 | | |
| seedlings | | | | |
| Average | 55% | 33% | 0.55 | 1 |

### Exogenous Auxin Application Induces PDLP5 Expression

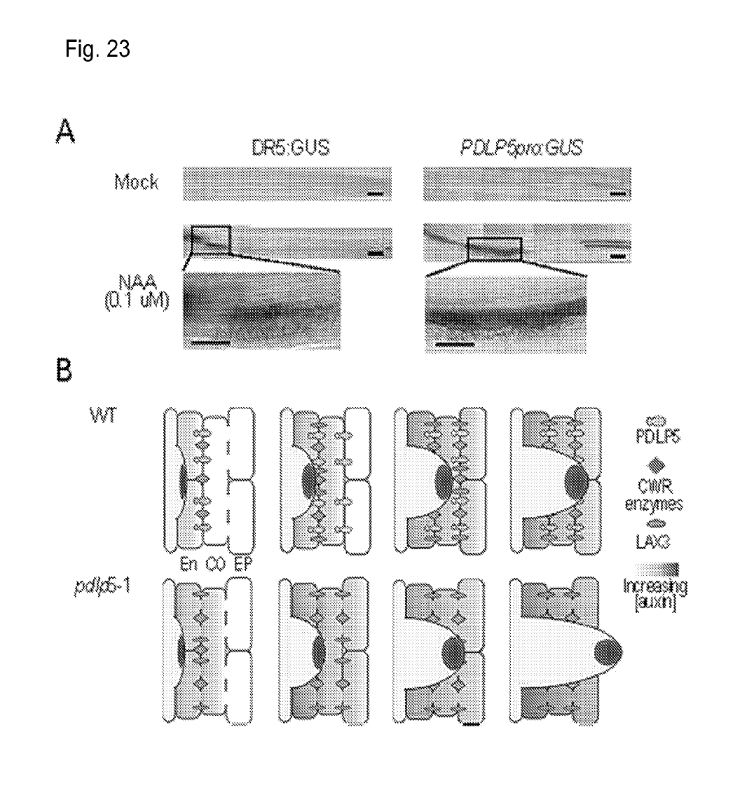

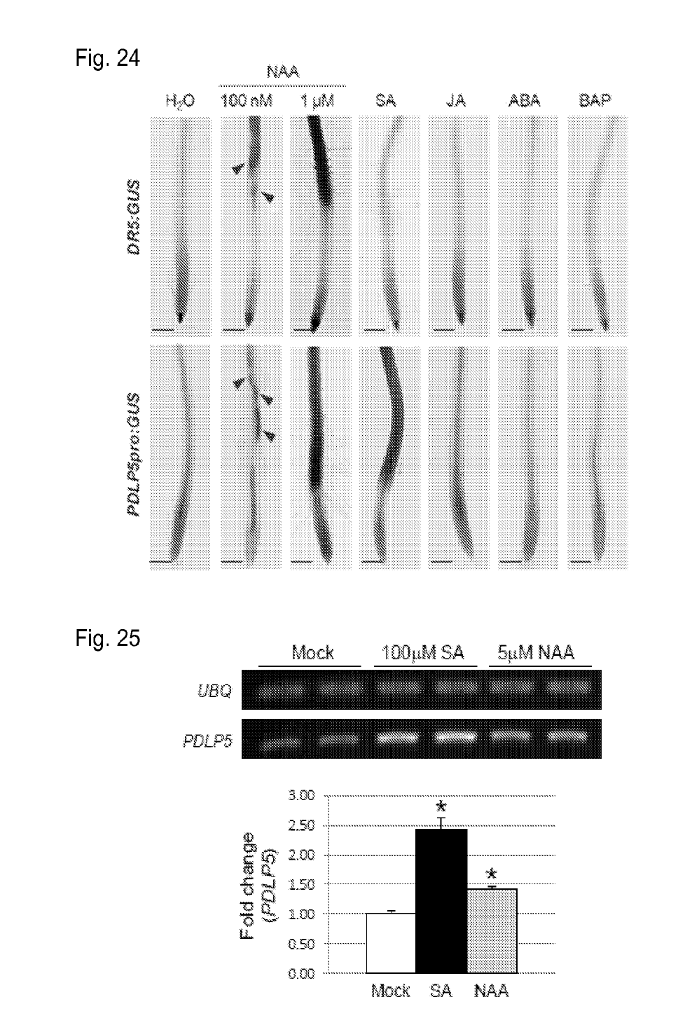

To further test that PDLP5pro responds to auxin, we treated 7-day-old PDLP5pro:GUS seedlings with the auxin analog 1-napthalene acetic acid (NAA). Similar to the DR5:GUS seedlings treated with 100 nM NAA, PDLP5pro:GUS expression occurred in the cells above the ectopically induced LRP that are formed closer to the primary root tip upon exogenous auxin treatment, which do not occur in the mock treated controls (FIG. 23A and FIG. 24). However, whereas DR5:GUS staining was intensified at the root meristem, exogenous auxin-induced PDLP5pro:GUS expression was again completely excluded from the root tip (FIG. 24). Consistent with previous findings in leaf tissue (Lee et al. 2011 Plant Cell 23(9):3353-3373), SA application also induces PDLP5pro:GUS expression in roots albeit in a diffusive manner covering most of the root tissue except for the root tip (FIG. 24). Other hormones such as cytokinin, ABA, or JA did not have any effect on PDLP5pro:GUS expression. Induction of PDLP5 at the transcript level in the roots by NAA and SA treatment was also confirmed by RT-PCR (FIG. 25).

### Discussion

Auxin exporters and importers, such as PIN1, PIN3, and the AUX/LAX family, are considered major players during LRP emergence for the tight control of directed auxin movement and maxima formation (Peret et al. 2012 Plant Cell 24(7):2874-2885; Marhavy et al. 2013 EMBO J32(1):149-158; Swamp and Peret, 2012 Front Plant Sci 3:225; Laplaze et al. 2007 Plant Cell 19(12):3889-3900; Ditengou et al. 2008. Proc Natl Acad Sci USA 105(48):18818-18823). Our study reveals that PD permeability, a previously unconsidered transcellular signaling route, also controls the LRP emergence process and led us to propose that normal progression of LR emergence requires coordinated PD closure via PDLP5 in cells overlying new organs.

The progression rate of secondary LRP emergence is significantly increased in pdlp5-1, and more tertiary roots develop earlier, resulting in a more branched root architecture compared to WT. Considering the enhanced PD permeability of this mutant, it is conceivable that auxin accumulation in overlying cells might be less effective due to a potential leakage of auxin from those cells. If this is the case, LRP may emerge sooner in pdlp5-1 through a positive feedback response. In this scenario, auxin leaks out of cells overlying LRP through PD and begins to accumulate in neighboring cells, triggering auxin influx gene expression and earlier cell separation via auxin-dependent CWR enzyme activity (Swamp et al. 2008 Nat Cell Biol 10(8):946-954; Peret et al. 2009 Trends Plant Sci 14(7):399-408; Lucas et al. 2013 Proc Natl Acad Sci USA. 110(13):5229-34; Peret et al., 2009 J Exp Bot 60(13):3637-3643); meanwhile, the leaking cells begin drawing more auxin from the shoot to attempt to compensate for the auxin diffusion through PD, and higher auxin levels lead to LRP growth promotion. A stark contrast was found in PDLP5OE seedlings, in which the LRP were much slower to emerge through overlying cell layers, significantly delaying not only emergence but overall LRP numbers and root lengths. Interestingly, in lax3, where LRP emergence is also inhibited, this situation brings about an apparent positive feedback, drawing more auxin from the shoot to accumulate in the root pericycle, and hence promoting more LRP initiation than in WT (Gonzalez-Carranza et al. 2007 J Exp Bot 58(13):3719-3730). We saw no such increase in LRP initiation in PDLP5OE roots, though this could be due to the overall reduction in auxin in PDLP5 roots, especially in the LRP.

Many genes that play critical roles during LR emergence are under spatiotemporal control for their expression. For example, expression of some CWR enzymes that are dependent on LAX3 is specific to Co or Ep cells, such as AIR3, PG, XTR6, and the peptide ligand IDA and receptors HAE/HSL3 (Swamp et al. 2008 Nat Cell Biol 10(8):946-954; Gonzalez-Carranza et al. 2007 J Exp Bot 58(13):3719-3730; Kumpf et al. 2013 Proc Natl Acad Sci USA. 110(13):5235-40; Neuteboom et al. 1999 Plant Mol Biol 39(2):273-287; Vissenberg et al. 2005 J Exp Bot 56(412):673-683), while others are expressed in En cells (Kong et al. 2013 Plant Cell Physiol. 54(4):609-21), depending on which specific transcription factors are involved. The PDLP5 expression pattern is unique in that it is expressed sequentially in all three root layers. The fact that the LAX3pro:LAX3-YFP signal accumulation is expedited in the overlying Co cells of pdlp5-1 while delayed in PDLP5OE suggests that PDLP5 expression likely precedes or occurs in parallel with LAX3 expression controlled by IAA14 and ARF7/19 (Swamp et al. 2008 Nat Cell Biol 10(8):946-954′ Peret et al. 2009 Trends Plant Sci 14(7):399-408).

Regardless of the transcriptional machinery, PDLP5 induction by auxin in cells overlying LRP likely imposes a transient, negative feedback to auxin import, acting as an intercellular stopcock during LR emergence to allow auxin accumulation within target cells to better ensure expression is limited very specifically to those. This regulatory mechanism may be necessary because hormones often have a very low threshold for initiating genetic responses within cells, and hence a small amount of auxin diffusion could be enough to trigger untimely downstream gene activation in overlying cells. The role of PDLP5 within En and Co cells could be especially critical to prevent early auxin leakage, as the presence of the water-impermeable Casparian strip would mean that the only route auxin could diffuse before loosening of the cell wall would be through PD. However, another possibility to consider in terms of the function of PDLP5 within these cells is related to its known function in inducing basal immunity (Marhavy et al. 2013 EMBO J32(1):149-158). The overlying cells, as they separate, could become vulnerable to infection by pathogenic soil microbes, and thus perhaps a well-coordinated timing of PDLP5 expression in these cells during LRP emergence is critical to ensure that the overlying cells do not separate before they are pre-primed for immunity.

In summary, we propose a model for the role of auxin-controlled PD-restriction via PDLP5 during LR emergence (FIG. 23B). In WT roots, auxin maxima formation at founder cells up-regulates PDLP5 expression in the En cells. PDLP5 up-regulation restricts PD permeability, preventing the passive diffusion of auxin from overlying En cells, activating SHY2-dependent CWR enzyme expression. Following En cell separation, auxin from the emerging LR tip can now move apoplastically into the overlying Co cells, where LAX3-controlled auxin influx ensures optimal auxin accumulation. PDLP5 again prevents uncontrolled diffusion of the auxin out of Co cells, which facilitates auxin accumulation and LAX3-dependent CWR. The combined efforts of auxin influx proteins and PD blockage focus the auxin maxima and cell separation within these target cells during LR emergence. In contrast, the absence of PDLP5 in pdlp5-1 roots allows diffusion of auxin through PD into overlying cells. This situation does not delay progression of LR emergence but rather accelerates it, by stimulating expression of genes like LAX3 that draw more shoot-derived auxin into cells overlying new organs. Dissection of the molecular mechanisms by which auxin controls PD permeability and how this event brings about a feedback regulation will be a necessary next step in order to further elucidate the role of PD during LRP emergence.

### Materials and Methods

### Plant Materials, Growth Conditions, and Genetic Crosses

All Arabidopsis thaliana genotypes were in the Col-0 genetic background, except for shy2-2 in Ler, and iaa28-1 in Ws. Seedlings were grown vertically in 0.5X MS agar under a continuous light at 22° C. Plants in soil were grown in a 16 hr light, 22° C. All the genetic crosses (see Table 5) were selfed, and genotyped to identify homozygous mutations when necessary. Genomic DNA was isolated from segregating F2 plants followed by PCR analyses using gene-specific primers (see Table 6).

TABLE 5
Genetic crosses used in this study.

| Maternal line | Paternal line | Cross used in study |
|---|---|---|
| PDLP5pro:GUS | shy2-2 | PDLP5pro:GUS × shy2-2 |
| | | (F2 showing root phenotype |
| | | and GUS staining) |
| PDLP5pro:GUS | iaa28-1 | PDLP5pro:GUS × iaa28-1 |
| | | (F2 showing root phenotype |
| | | and GUS staining) |
| LAX3pro:LAX3-YFP | pdlp5-1 | LAX3pro:LAX3-YFP × pdlp5-1 |
| | | (F3 homozygous for pdlp5-1, |
| | | segregating LAX3-YFP) |
| LAX3pro:LAX3-YFP | 35S:PDLP5 | LAX3pro:LAX3-YFP × |
| | | 35S:PDLP5 (F3 homozygous |
| | | for 35S:PDLP5, segregating |
| | | LAX3-YFP) |

TABLE 6
PCR Primers used.

| | | SEQ | |
|---|---|---|---|
| Primer name | Sequence (5′ → 3′) | ID NO: | Purpose |
| PDLP5gen Rv | TTTTGCATAGACGAAAAACATGG | 31 | genotyping |
| PDLP5gen Fw | TGGATCTTACAGGACAGGTGG | 32 | |
| SAIL LB1 | CCTTTTCAGAAATGGATAAATAGCCTTGCTTCC | 33 | |
| UBI Fw | GGAAGACCATAACCCTTGAGGTTG | 34 | RT-PCR |
| UBI Rv | TCTTAGCACCACCACGGAGA | 35 | |
| PDLP5 Fw | CCGCTACGCCAACTTCACAG | 36 | RT-PCR |
| PDLP5 Rv | CTTCTCTCCTTCATGACCAAAGT | 37 | |
| KH318 | GTTCACTCAAATCTATAATAGGCATAGG | 38 | PDLP5 promoter −2341 to −2260 |
| KH319 | CGACAAATTGTGGAACTCTTTCA | 39 | |
| KH320 | GGTAGAGGCTAACGAATTCACA | 40 | PDLP5 promoter −394 to −285 |
| KH321 | GTGCGTCTATCCATTACAACTTTC | 41 | |
| KH69 | TGCATTGGTACACAGGTGAGGGAA | 42 | TUB3 (At5g62700) control |
| KH70 | AGCCGTTGCATCTTGGTATTGCTG | 43 | primer pair: exon 3 (+1756 to 1863) |
| KH322 | CACAATGTTTGGCGGGATTGGTGA | 44 | Actin12 (At3g46520) control |
| KH323 | TGTACTTCCTTTCCGGTGGAGCAA | 45 | primer pair: exon 3 (+1095 to 1199) |

### GUS Assay and LRP Quantification

GUS solution (100 mM sodium phosphate buffer, pH 7.0, 10 mM EDTA, 0.5 mM each potassium ferrocyanide and potassium ferricyanide, 1.24 mM X-Gluc, and 0.1% Triton X-100) was vacuum-infiltrated into plant tissue for five minutes, then removed from vacuum and incubated in 37° C. for 3 to 12 hrs, followed by a series of ethanol washes. Stained tissues were imaged using Zeiss Axioskop 2 microscope. LRP were quantified by counting both the emerged LR and unemerged LR, as determined by DRS::GUS staining of the primordia, under a dissecting microscope (1.2× magnification). LRP stages were determined by examining ethanol-cleared, GUS-stained tissue using 40× water lens.

Chromatin Immunoprecipitation and qPCR Analyses

ChIP assay was performed on Col-0 and a knock-out allele, arf19-1 (Fukaki et al. 2002) Plant J29(2):153-168), using 2-3 g root tissue pre-treated with 1 μM NAA and fixed under vacuum with 1% formaldehyde for 15 minutes. Nuclei were extracted following the protocol described previously (Bowler et al. 2004 Plant J39(5):776-789) and ChIP was perfomed, using an anti-ARF19 anitbody following the method basically as described previously (Hill et al. 2008 Plant J53(1):172-185; Nakaminami et al. 2009 J Exp Bot 60(3): 1047-1062). Briefly, 200 μl of sonciated chromatin was added to 1 ml Immunoprecipitation Buffer (50 mM Hepes, pH 7.5, 150 mM KCl, 5 mM MgC12, 0.1% Triton X-100) and incubated along with 3 μg of anti-ARF19 at 4° C. on a slow-moving rota for 4 hrs. Protein G Dynabeads® (Invitrogen) were added to the chromatin and antibody mix and further incubated at 4° C. overnight. The magnetic beads were washed 4 times for 1 h with Immunoprecipitation Buffer, and twice with H₂O, followed by elution and reverse cross-linking at 95° C. in 0.5 M NaCl solution and Proteinase K treatment overnight at 55° C. Input and ARF19 immunoprecipiated DNA was used for qPCR with SYBR green master mix and 1 μM each of forward and reverse oligonucleotides (see Table 6) All qPCR reactions were performed as quadruplicate triplicate technical replicates using a Light Cycler 480 qPCR machine and are representative of three biological repeats. Oligos were designed to two regions of the PDLP5/HWI1 (At1g70690) promoter. The promoter region −2341 to −2260 (relative to ATG start codon) includes a canonical site (TGTCTC; SEQ ID NO:12). The promoter region −394 to −285 has several core (minimal) binding elements TGTC/GACA (SEQ

ID NO:13/48; see FIG. 26). Fold enrichment is calculated as the amount of promoter fragment immunoprecipted relative to the non-immunoprecipitaed input chromatin, normalised using primers designed to the 3′ end of the Tubulin3 gene (see Table 6). Similar results were obtained using two different internal controls (sites within the TUB3 and ACTIN12 genes that not believed to be bound by ARFs).

### Gravistimulation and Confocal Microscopy

Arabidopsis seedlings expressing LAX3::LAX3:YFP in the desired background were grown as described above for three days, followed by gravistimulation by turning the plates 90° . The cortical cell fluorescence at the root bend was monitored at different time points using a Zeiss LSM 780 confocal upright light microscope using a W Plan-Apochromat 20×/1.0 DIC M27 75 mm objective and the 415-nm excitation line of an argon laser with 520-550 nm band pass emission filter. Images are presented as 3-D composites of 30 μm-thick z-stacks.

### Example 3

### Transformation of Maize with Modified PDLP5 Protein Using Particle Bombardment

Maize plants can be transformed to express a modified PDLP5 protein from Arabidopsis, such as PDLP5-m5, or corresponding homologs derived from various species in order to examine the resulting phenotype.

A polynucleotide encoding the modified PDLP5 protein can be cloned into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

The recombinant DNA construct can then be introduced into corn cells by particle bombardment. Techniques for corn transformation by particle bombardment have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Constructs that result in a significant delay in wilting or leaf area reduction, yellow color accumulation and/or increased growth rate during drought stress is considered evidence that the Arabidopsis-derived protein functions in maize to enhance drought tolerance.

### Example 4

### Transformation of Maize Using Agrobacterium

Maize plants can be transformed to express a modified PDLP5 protein from Arabidopsis, such as PDLP5-m5, or corresponding homologs derived from various species in order to examine the resulting phenotype.

A polynucleotide encoding a modified PDLP5 protein can be cloned into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium innoculation, co-cultivation, resting, selection and plant regeneration.

Transgenic T0 plants can be regenerated and their phenotype determined. T1 seed can be collected.

Furthermore, a recombinant DNA construct containing a recombinant DNA construct encoding a modified PDLP5 protein from Arabidopsis can be introduced into an elite maize inbred line either by direct transformation or introgression from a separately transformed line.

### Example 5

### Yield Analysis of Maize Lines with a Modified PDLP5 Protein

A recombinant DNA construct encoding a modified PDLP5 protein from Arabidopsis, such as PDLP5-m5, or a homologous protein derived from another species, can be introduced into an elite maize inbred line either by direct transformation or introgression from a separately transformed line.

Transgenic plants, either inbred or hybrid, can undergo more vigorous field-based experiments to study yield enhancement and/or stability under well-watered and water-limiting conditions.

Subsequent yield analysis can be done to determine whether plants that contain the modified PDLP5 protein have an improvement in yield performance under water-limiting conditions, when compared to the control plants that do not contain the modified PDLP5 protein. Specifically, drought conditions can be imposed during the flowering and/or grain fill period for plants that contain a modified PDLP5 protein and the control plants. Reduction in yield can be measured for both. Plants containing the a modified PDLP5 protein may have less yield loss relative to the control plants, for example, at least 25%, at least 20%, at least 15%, at least 10% or at least 5% less yield loss.

The above method may be used to select transgenic plants with increased yield, under water-limiting conditions and/or well-watered conditions, when compared to a control plant not including said recombinant DNA construct. Plants containing a modified PDLP5 protein may have increased yield, under water-limiting conditions and/or well-watered conditions, relative to the control plants, for example, at least 5%, at least 10%, at least 15%, at least 20% or at least 25% increased yield.

### Example 6

### Preparation of Soybean Expression Vectors and

### Transformation of Soybean with a Modified PDLP5 Protein

Soybean plants can be transformed to express a modified PDLP5 protein from Arabidopsis, such as PDLP5-m5, or corresponding homologs from various species in order to examine the resulting phenotype.

A polynucleotide encoding a modified PDLP5 protein can be cloned into the PHP27840 vector (PCT Publication No. WO/2012/058528) such that expression of the protein is under control of the SCP1 promoter (International Publication No. 03/033651).

Soybean embryos may then be transformed with the expression vector including sequences encoding the instant polypeptides. Techniques for soybean transformation and regeneration have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Constructs that result in a significant delay in wilting or leaf area reduction, yellow color accumulation and/or increased growth rate during drought stress will be considered evidence that a modified PDLP5 protein from Arabidopsis functions in soybean to enhance drought tolerance.

Soybean plants transformed with a Modified PDLP5 Protein can then be assayed under more vigorous field-based studies to study yield enhancement and/or stability under well-watered and water-limiting conditions.

The complete invention of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the invention of the present application and the invention(s) of any document incorporated herein by reference, the invention of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1.-25. (canceled)

26. A recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail.

27. The recombinant DNA construct of claim 26, wherein the polynucleotide comprises
(a) a nucleotide sequence encoding a modified PDLP5 protein with drought tolerance activity, wherein the modified PDLP5 protein has an amino acid sequence of at least 80% and less than 100% sequence identity when compared to SEQ ID NO:4, based on the Clustal V method of alignment with pairwise alignment default parameters of KTUPLE=1, GAP PENALTY=3, WINDOW=5 and DIAGONALS SAVED=5; or
(b) the full complement of the nucleotide sequence of (a).

28. The recombinant DNA construct of claim 26 wherein the nucleotide sequence comprises SEQ ID NO:5.

29. The recombinant DNA construct of claim 26, wherein the regulatory element comprises a promoter selected from the group consisting of a constitutive promoter, a tissue-specific promoter, and a physically inducible promoter that stimulates expression in response to exposure to plant stress.

30. The recombinant DNA construct of claim 26, wherein the modified PDLP5 protein comprises a modification of any one cysteine residue, any two cysteine residues, or all three cysteine residues, said one, two or three cysteine residues selected from C288, C289, and C298 of A. thaliana PDLP5 (SEQ ID NO:4) or an analogous cytosolic cysteine residue in a homologous PDLP5 protein.

31. The recombinant DNA construct of claim 26, wherein the amino acid sequence of the modified PDLP5 protein comprises PDLP5-m5 (SEQ ID NO:6).

32.-33. (canceled)

34. A method of producing a plant that exhibits at least one trait selected from the group consisting of increased drought tolerance, increased yield, increased biomass, increased cold tolerance, early flowering, and altered root architecture, and a combination thereof, wherein the method comprises:
(a) introducing into a plant cell the recombinant DNA construct of claim 26;
(b) growing a transgenic plant from the plant cell of (a), wherein the transgenic plant comprises in its genome the recombinant DNA construct; and
(c) obtaining a progeny plant derived from the transgenic plant of (b), wherein said progeny plant comprises in its genome the recombinant DNA construct and exhibits at least one trait selected from the group consisting of increased drought tolerance, increased yield, increased biomass, increased cold tolerance, early flowering, and altered root architecture, and a combination thereof, when compared to a control plant not comprising the recombinant DNA construct.

35. A method of producing a plant that exhibits at least one trait selected from the group consisting of increased drought tolerance, increased yield, increased biomass, increased cold tolerance, early flowering, altered root architecture, and a combination thereof, wherein the method comprises growing a plant from a seed comprising the recombinant DNA construct of claim 26, wherein the plant exhibits at least one trait selected from the group consisting of increased drought tolerance, increased yield, increased biomass, increased cold tolerance, early flowering, and altered root architecture, and a combination thereof, when compared to a control plant not comprising the recombinant DNA construct.

36. A method of making a plant wherein the endogenous PDLP5 has been modified, wherein the method comprises the steps of:
(a) introducing a mutation into the endogenous PDLP5 gene; and
(b) detecting the mutation;
wherein step (a) is performed using CRISPR technology.

37.-41. (canceled)

42. A plant comprising the recombinant DNA construct of claim 26, wherein said plant exhibits at least one trait selected from the group consisting of increased drought tolerance, increased yield, increased biomass, increased cold tolerance, early flowering, altered root architecture, and a combination thereof, when compared to a control plant not comprising said recombinant DNA construct.

43. The plant of claim 42, wherein said plant is selected from the group consisting of maize, soybean, sunflower, sorghum, canola, wheat, alfalfa, cotton, rice, barley, millet, sugar cane and switchgrass.

44. The plant of claim 42, wherein the modified PDLP5 protein comprises a modification of any one cysteine residue, any two cysteine residues, or all three cysteine residues, said one, two or three cysteine residues selected from C288, C289, and C298 of A. thaliana PDLP5 (SEQ ID NO:4) or an analogous cytosolic cysteine residue in a homologous PDLP5 protein.

45. A seed of the plant of claim 42, wherein said seed comprises a recombinant DNA construct comprising a polynucleotide operably linked to at least one regulatory element, wherein said polynucleotide encodes a modified PDLP5 protein having an amino acid sequence of at least 80% sequence identity, based on the Clustal V or Clustal W method of alignment, when compared to SEQ ID NO:4 (A. thaliana wild-type PDLP5) or SEQ ID NO:6 (PDLP5-m5), provided that the modified PDLP5 protein has 0, 1 or 2 cysteines in the cytosolic C-terminal tail, and wherein a plant produced from said seed exhibits at least one trait selected from the group consisting of increased drought tolerance, increased yield, increased biomass, increased cold tolerance, early flowering, altered root architecture, and a combination thereof, when compared to a control plant not comprising said recombinant DNA construct.

46. A method of producing a plant, wherein the method comprises growing a plant from the seed of claim 45, wherein the plant exhibits at least one trait selected from the group consisting of increased drought tolerance, increased yield, increased biomass, increased cold tolerance, early flowering, altered root architecture, and a combination thereof, when compared to a control plant not comprising the recombinant DNA construct.

47. A method of increasing drought tolerance in a plant, wherein the method comprises:
(a) introducing into a regenerable plant cell the recombinant DNA construct of claim 26;
(b) regenerating a transgenic plant from the regenerable plant cell of (a), wherein the transgenic plant comprises in its genome the recombinant DNA construct; and
(c) obtaining a progeny plant derived from the transgenic plant of (b), wherein said progeny plant comprises in its genome the recombinant DNA construct and exhibits increased drought tolerance when compared to a control plant not comprising the recombinant DNA construct.

48. A method of producing oil or a seed by-product, or both, from a seed, the method comprising extracting oil or a seed by-product, or both, from a seed that comprises the recombinant DNA construct of claim 26.

49. A method of selecting for a plant that exhibits at least one trait selected from the group consisting of increased drought tolerance, increased yield, increased biomass, increased cold tolerance, early flowering, altered root architecture, and a combination thereof, wherein the method comprises:
(a) obtaining a transgenic plant, wherein the transgenic plant comprises in its genome the recombinant DNA construct of claim 26;
(b) growing the transgenic plant of part (a) under conditions wherein the polynucleotide is expressed; and
(c) selecting the transgenic plant of part (b) that exhibits at least one trait selected from the group consisting of increased drought tolerance, increased yield, increased biomass, increased cold tolerance, early flowering, altered root architecture, and a combination thereof, when compared to a control plant not comprising the recombinant DNA construct.

50. A plant or seed comprising the recombinant DNA construct of claim 26, wherein the modified PDLP5 protein exhibits semi-dominant negative gain-of-function activity when compared to a wild-type PDLP5 protein.

51. The plant or seed of claim 50, wherein the modified PDLP5 protein comprises PDLP5-m5 (SEQ ID NO:6).

52. The plant or seed of claim 50, wherein the plant comprises an endogenous PDLP5 protein, and the modified PDLP5 protein comprises a semi-dominant negative gain-of-function PDLP5 protein.
